(19) United States
(12) Patent Application Publication
(10) Pub. No.: US 2010/0130432 A1
(43) Pub. Date: May 27, 2010

(54) Non-Natural Amino Acids and Neurotensin Analogues Thereof

(75) Inventors: Justin O. Brower, Ladson, SC (US); Thomas A. Dix, Mt. Pleasant, SC (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Argolyn Bioscience Inc. and Medical University of South Carolina [MUSC]

(21) Appl. No.: 12/086,528

(22) PCT Filed: Dec. 15, 2006
(86) PCT No.: PCT/US2006/047860

§ 371 Date: Oct. 13, 2009

Related U.S. Application Data
(60) Provisional application No. 60/751,165, filed on Dec. 16, 2005.
(60) Provisional application No. 60/814,240, filed on Jun. 16, 2006.
(60) Provisional application No. 60/814,355, filed on Jun. 16, 2006.

(51) Int. Cl. A61K 38/10; C07D 233/48; A61K 38/08; A61K 31/505; A61K 31/4164; A61K 31/4168; A61K 31/195; A61K 31/155; C07K 7/08; C07K 7/06; C07D 239/14; A61K 8/64; C07D 233/02; C07C 279/14; C07C 229/26; A61P 3/04; A61P 35/00; A61P 3/10; A61P 25/18; A61P 25/30; A61Q 1/06; A61Q 1/08; A61Q 1/12; C12Q 1/02

(52) U.S. Cl. 514/14; 548/331.5; 514/17; 514/275; 514/385; 514/392; 514/564; 514/565; 530/327; 530/329; 530/330; 544/332; 562/560; 562/561; 562/562; 435/29

(57) ABSTRACT

This invention relates to non-natural desamino amino acid compounds, methods of making, and peptides containing these compounds as their N-terminus moieties. A preferred example is neurotensin (8-13) in which the N terminus is an alpha desamino N,N dimethyl homolysine residue.

This patent document claims the benefit of priority to U.S. Provisional Patent Application Ser. No. 60/751,165, filed on Dec. 16, 2005; U.S. Provisional Patent Application Ser. No. 60/814,240, filed on Jun. 16, 2006; and U.S. Provisional Patent Application Ser. No. 60/814,355, filed on Jun. 16, 2006; all of which are herein incorporated by reference.

### BACKGROUND OF THE INVENTION

The influence that some non-natural amino acids have on the structural and biological activity of peptides has been briefly studied. For example, Moore et al. (Can. J. Biochem. 1978, 56, 315) disclosed the effect of the basic amino acid side chain length and the penultimate residue on the hydrolysis of benzoyldipeptides by carboxylicpeptidase B¹ (CPB). Non-natural amino acids including homolysine and homoarginine were incorporated into small peptide chains, and the kinetic parameters were determined for the CPB catalyzed hydrolysis of the peptide. Also, Lindeberg et al. (Int. J. Peptide Protein Res. 1977, 10, 240) disclosed the synthesis of 1-deamino-4-L-valine-8-DL-homolysine-vasopressin and protected 1-deamino-4-L-valine-8-D-lysine-vassopressin in which non-natural amino acids were incorporated. The non-natural amino acids were formed by addition of a methylene group to lysine and arginine to generate the non-natural amino acids homolysine and homoarginine, respectively. The study revealed that peptides with homolysine and homoarginine reduced the antidiuretic activity of the peptides.

Naturally occurring endogenous peptides are ideal drug candidate leads by virtue of their myriad activities in promoting and regulating biological processes. Inherent in the chemistry and biology of peptides, however, are several factors that also make them poor drug candidates. Peptides most often exert localized effects and are rapidly degraded within the body. In addition, most peptides are unable to cross biological membranes, including the small intestine and blood brain barrier (BBB). Finally, peptides often bind to more than one receptor or receptor subtype, thus rarely showing the selectivity required of a viable drug candidate. Therefore, for a peptide to become a viable drug candidate, improvements in blood stability, receptor selectivity, and barrier crossing should be made without eliminating inherent binding affinity.

Numerous strategies have been developed as methods for improving peptide stability, including N- and C-terminal modifications to prevent exopeptidase activity, amide backbone modifications, and the introduction of conformational constraints to disguise peptides from peptidase degradation. Other therapeutic compounds employ a prodrug moiety intended to modify its overall hydrophobicity, which can result in the compound crossing biological membranes. In this case, the compound is cleaved into its active component by endogenous enzymes. While each of these strategies has been used to improve peptides as drug candidates, a universal solution for creating stable, receptor-selective peptides that cross biological barriers has not been discovered.

Consequently, there is a need in the art for non-natural amino acids and for peptides incorporating such acids to achieve superior effects, such as, for example, improved diagnostic or disease fighting activity. Thus, the non-natural amino acid concept could be applied to development of new peptide pharmaceuticals. One example of such a development is the application to neuropeptides such as neurotensin. Neurotensin (NT) is a 13-amino acid residue peptide found primarily in the brain. It has multifunctional activities manifested through binding and activation of two neurotensin receptors (NTRs), NTR-1 and NTR-2 (see Carraway & Leeman, J. of Biol. Chem. 248: 6854 (1973)). Although full activity of NT resides in its C-terminal six amino acid sequence (NH₂-Arg⁽⁸⁾-Arg⁽⁹⁾-Pro⁽¹⁰⁾-Tyr⁽¹¹⁾-Ile⁽¹²⁾-Leu⁽¹³⁾-COOH, designated NT[8-13], see Carraway & Leeman, J. Biol. Chem. 250:1907 (1975)), the C-terminal six amino acid sequence does not have activity when administered IP or orally due to its instability in blood and inability to cross the blood brain and/or gut barrier.

### SUMMARY OF CERTAIN EMBODIMENTS OF THE INVENTION

The present invention concerns alpha-desamino amino acid compounds (desamino amino acid compounds) that are capable of carrying positively charged side chains, their synthesis, their application as substitutes for natural amino acid moieties of biologically active peptides and the resulting peptides as well. In particular, alpha-desamino arginine, lysine and ornithine as well as their substituted and derivatized side chain analogs constitute preferred embodiments of the invention. These desamino amino acid compounds can be substituted for arginine and/or lysine moieties in any known, biologically active peptide such that the substituted peptide will be truncated at the substitution position. Alternatively, these desamino amino acid compounds can be coupled to the amino group of the N-terminus of any known biologically active peptide to produce an extended peptide. The truncated and extended peptides have significant biological selectivity and biological half lives owing to their resistance toward amino peptidase degradation.

In a first aspect, the invention relates to a non-natural desamino amino acid compound having Formula I:

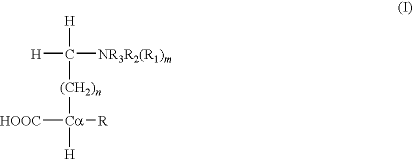

wherein

n is an integer of from 0 to 5, preferably, 2 to 5;

m is zero or an integer of 1;

R is H or an organic substituent such as a straight or branched chain alkyl group of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination;

R₁, R₂, and R₃ are, independently, hydrogen or branched or straight chain alkyl, alkenyl or alkynyl of C₁-C₆ or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination and with the proviso that a maximum of two of R₁, R₂, and R₃ may be selected to be the aromatic, substituted aromatic, heteroaromatic or substituted heteroaromatic group, and provided that when m is 0 or 1 and n is 0 to 5, R₁, R₂, and R₃ are not all H;

Cα is a carbon atom having either R or S stereochemistry if the substituent at R is an organic substituent;

or an ester, amide, alkyl amide or metal cation or ammonium salt of the carboxylic acid group thereof, or an organic or inorganic acid salt of the amine group thereof, or any combination thereof.

In a second aspect, the invention relates to a non-natural desamino amino acid compound of the formula II:

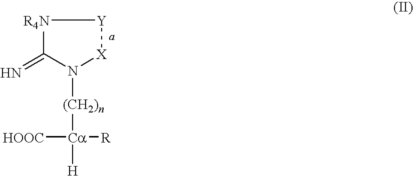

wherein

n is an integer of from 0 to 6, preferably, 2 to 5;

when dashed line a is not present, X and Y are independently, hydrogen or lower branched or straight chain alkyl, alkenyl or alkynyl of C₁-C₆;

when dashed line a is present, X—Y is (CH₂)_{z}, wherein z is an integer of from 1-8, preferably, 2 to 4;

R is H or an organic substituent such as a straight or branched chain alkyl group of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination;

R₄ is hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination, and;

C_{α} is a carbon atom and the stereochemistry at C_{α} is either R or S if the substituent at R is an organic substituent;

or an ester, amide, alkyl amide or metal cation or ammonium salt of the carboxylic acid group thereof, or an organic or inorganic acid salt of the amine group thereof, or any combination thereof.

A third aspect of the present invention relates to a non-natural desamino amino acid compound of the formula III:

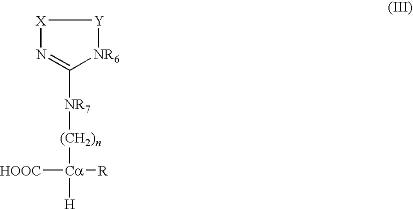

wherein

n is an integer of from 0 to 5, preferably, 2 to 5;

X—Y is (CH₂)_{z}, wherein z is an integer of from 0 to 6, preferably, 2 to 4;

R is H or an organic substituent such as a straight or branched chain alkyl group of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination;

R₆, and R₇ are, independently, hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination; and

C_{α} is a carbon atom and the stereochemistry at C_{α} is either R or S if the substituent at R is an organic substituent;

or an ester, amide, alkyl amide or metal cation or ammonium salt of the carboxylic acid group thereof, or an organic or inorganic acid salt of the amine group thereof, or any combination thereof.

A fourth aspect of the invention relates to a non-natural desamino amino acid compound of the formula IV:

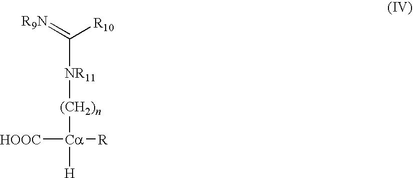

wherein

n is an integer of from 0 to 5, preferably, 2 to 4;

R is H or an organic substituent such as a straight or branched chain alkyl group of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination;

R₉, R₁₀, and R₁₁ are, independently, hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination and with the proviso that a maximum of two of R₉, R₁₀, and R₁₁ may be selected to be the aromatic, substituted aromatic, heteroaromatic or substituted heteroaromatic group; and

C_{α} is a carbon atom and the stereochemistry at C_{α} is either R or S if the substituent at R is an organic substituent;

or an ester, amide, alkyl amide or metal cation or ammonium salt of the carboxylic acid group thereof, or an organic or inorganic acid salt of the amine group thereof, or any combination thereof.

A fifth aspect of the invention relates to a non-natural desamino amino acid compound of the formula V:

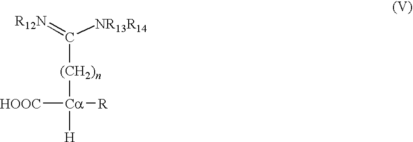

wherein

n is an integer of from 0 to 5, preferably, 2 to 4;

R is H or an organic substituent such as a straight or branched chain alkyl group of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination;

R₁₂, R₁₃, and R₁₄ are, independently, hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteraromatic group with one or two substituents selected from halogen, alkyloxy, carboxy, amide or alkyl in any combination and with the proviso that a maximum of two of R₁₂, R₁₃, and R₁₄ may be selected to be the aromatic, substituted aromatic, heteroaromatic or substituted heteroaromatic group; and

C_{α} is a carbon atom and the stereochemistry at C_{α} is either R or S if the substituent at R is an organic substituent;

or an ester, amide, alkyl amide or metal cation or ammonium salt of the carboxylic acid group thereof, or an organic or inorganic acid salt of the amine group thereof, or any combination thereof.

A further aspect of the invention relates to the addition of the non-natural desamino amino acid compounds of the invention to the N-terminus amino group of biologically active peptides or their substitution for naturally occurring congener amino acid moieties of biologically active peptides. Preferred congener moieties include arginine and/or lysine.

The addition to the N-terminus amino group of a known, biologically active peptide provides an extended peptide that has selective, long lasting biological activity of the same kind as the known, biologically active peptide. The addition can be accomplished by known methods for coupling acid and amine groups together to form amide bonds, including use of acyl azide coupling, carbodiimide coupling, acid ion exchange resin, triaminoboranes and enzyme coupling. A preferred method involves the use of an amino exopeptidase under conditions that promote the peptide bond formation. In some embodiments of the invention, the semisynthetic peptides are produced by substituting a non-natural amino acid compound for the N-terminal arginine residue of NT (8-13), e.g., ABS205, ABS207, ABS208, ABS210, ABS211, ABS212, ABS220, ABS225, ABS226, ABS227, ABS228, ABS230, ABS232, ABS234, or ABS239.

Preferred embodiments of the peptides on which the extended peptides are based include biologically active peptides useful for treatment or prevention of malconditions. A list of preferred categories and examples is included in the sections below. Some preferred categories include but are not limited to transcription factors, ligands for cellular receptors, hormones and extracellular binding peptides. Some preferred examples include but are not limited to enkephlin, LHRH and analogs, neuropeptides, glycoincretins, integrin and analogs, glucagons and glucagon-like peptides, antithrombotic peptides, cytokines and interleukins, transferrins, interferons, endothelins, natriuretic hormones, extracellular kinase ligands, angiotensin enzyme inhibitors, peptide antiviral compounds, thrombin, substance P, substance G, somatotropin, somatostatin, GnRH and analogues, secretin, bradykinin, vasopressin and analogues, insulin and analogs thereof, growth factors, as well as others. The extended peptide is formed by coupling the N-terminus amino group of a basis peptide to the carboxyl group of a desamino amino acid compound of the invention.

The substitution of desamino amino acid moiety for an arginine or lysine moiety of a biologically active peptide provides a truncated peptide having selective, long-lasting biological activity. Any known biologically active peptide having an arginine and/or lysine moiety within its amino acid sequence can serve as the basis for the corresponding truncated peptide. Beginning at that ARG or LYS moiety, the truncated peptide will have the same downstream sequence as the known, biologically active peptide but the upstream sequence will be absent. In addition, that ARG or LYS moiety will be exchanged for a desamino amino acid moiety, thus providing the truncated peptide. Several known biologically active peptides are penultimately formed as pro-peptides with an arginine or lysine moiety at the pro-peptide or precursor cleavage position, or are formed as final peptides containing an arginine or lysine moiety at a position that can be cleaved to provide an active truncated peptide. Trypsin is an enzyme specific for such cleavage points. Examples include glucagon-like peptide, neurotensin, proinsulin, and thrombin. The truncated versions of these examples with a desamino amino acid compound substituted for the arginine or lysine moiety provide selective, long-lasting biological activity.

A further aspect of the invention includes pharmaceutical and cosmetic compositions of the desamino amino acid compound, of the extended or truncated peptide, and combinations thereof. Unit dosage forms and biologically effective formulations of the pharmaceutical compositions are included. The cosmetic formulations include appropriate oil, creme, wax or aqueous base cosmetic carriers.

Yet another aspect of the invention includes methods of screening, diagnosis and treatment using the desamino amino acid compounds of the invention and/or the addition or truncated peptides.

One embodiment of the invention is a truncated neurotensin peptide having a desamino amino acid as its N-terminus amino acid moiety.

The invention also provides processes and intermediates disclosed herein that are useful for preparing compounds of the inventions, such as compounds of Formula I, II, III, IV and/or V and peptides that contain such compounds. One class of such intermediates includes the N-protected or carboxyl protected or N- and carboxyl protected compounds of Formulas I, II, III, IV and V. These protected intermediates are described in detail in the following sections of the application. Another class of such intermediates includes the carboxylate salts of the compounds of Formulas I, II, III, IV and V, the organic or inorganic acid amine salts of those compounds and the double salts (carboxylate, amine salts).

### DEFINITIONS OF THE INVENTION

As used in the specification and the appended claims, the singular forms “a,” “an” and “the” include plural referents unless the context clearly dictates otherwise.

Variables, such as R₁-R₃, n, z, X, Y, C_{α} and C_{β}, throughout the application are the same variables as defined herein unless stated to the contrary.

The term “alkyl” as used herein refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Preferred alkyl groups herein contain from 1 to 6 carbon atoms.

The term “alkenyl” as used herein refers to a hydrocarbon group of 2 to 24 carbon atoms, with preferred groups within this class containing 2 to 6 carbon atoms, and structural formula containing a carbon-carbon double bond.

The term “alkynyl” as used herein refers to a hydrocarbon group of 2 to 24 carbon atoms, with preferred groups within this class containing 2 to 6 carbon atoms, and a structural formula containing a carbon-carbon triple bond.

As used herein, especially in reference to alkyl, alkenyl and alkynyl, unless defined otherwise, the term “lower” refers to a moiety having from 1 to 6 carbon atoms, preferably 1 to 4 carbon atoms, more preferably 1 to 2 carbon atoms.

The term “alkylating agent” as provided herein is a compound with the structural formula RX, where R is an alkyl, alkenyl or alkynyl group as previously described, and X, which is preferably a halide such as chloride, bromide or iodide.

As used herein, the term “non-natural amino acid” refers to an organic compound that is a congener of a natural amino acid in that it has a structure similar to a natural amino acid so that it mimics the structure and reactivity of a natural amino acid. The non-natural amino acid as defined herein generally increases or enhances the properties of a peptide (e.g., selectivity, stability) when the non-natural amino acid is either substituted for a natural amino acid unit of a peptide or otherwise incorporated into a peptide.

As used herein, the term “peptide” refers to a class of compounds composed of amino acids chemically bound together. In general, the amino acids are chemically bound together via amide linkages (—CONH—); however, the amino acids may be bound together by other chemical bonds known in the art. For example, the amino acids may be bound by amine linkages. Peptide as used herein includes oligomers of amino acids and small and large peptides, including polypeptides.

As used herein, the term “activity” refers to a biological activity.

As used herein, the term “pharmacological activity” refers to the inherent physical properties of a peptide or polypeptide. These properties include but are not limited to half-life, solubility, and stability and other pharmacokinetic properties.

The term “organic acid salt” as used herein refers to the salt form of an amine group with an alkyl or aryl C₁-C₉ carboxylic, sulfonic, or phosphoric acid.

The term “inorganic acid salt” as used herein refers to the salt form of an amine group with a mineral acid such as hydrochloric, sulfuric, sulfonic, phosphoric, nitric, nitrous, or hydrobromic acid.

The term “aromatic of C₆ to C₁₈” as used herein refers to an aromatic hydrocarbon such as phenyl, naphthyl, anthracenyl, or an arylalkyl hydrocarbon such as benzyl, phenethyl or naphthylmethylenyl.

The term “heteraromatic of C₄ to C₁₈ and of one or two heteroatoms selected from oxygen, sulfur and nitrogen in any combination” as used herein refers to a heteroaromatic hydrocarbon containing one or two heteroatoms or an alkyl heteroaromatic hydrocarbon such as thienyl, furyl, pyrrolyl, azathienyl, azafuryl, pyridinyl, thiapyridinyl, pyrazinyl, methylenylpyridinyl, ethylenylpyridinyl, methylenylpyrrolyl and the like.

The term “R or S” in connection with stereochemistry has the ordinary meaning designating the optical isomerism of the selected carbon. R in this context should not be confused with R as a substituents.

The chemical, pharmaceutical and biological terms used herein follow the ordinary and customary meanings one of skill, such as a Ph.D. researcher in the field would attribute to them. Such meanings may be found in appropriate technical dictionaries and treatises such as but not limited to “Hawley's Condensed Chemical Dictionary”, 11^{th} Ed., Sax and Lewis Editors, Van Nostrand Reinhold Publishing, New York, N.Y. 1987; “Concise Chemical and Technical Dictionary”, 4^{th} enlarged Ed. Bennett Editor, Chemical Publishing Inc., New York, N.Y., 1986, “The Merck Index” 11^{th} and succeeding Editions, Merck & Co. Rahway, N.J. 1989 and more recent; “Advanced Organic Chemistry” 4^{th} Ed., J. March, Wiley Interscience, New York, N.Y. 1992; “Concise Dictionary of Biomedicine and Molecular Biology”, Pei-Show Juo Ed., CRC Press, New York, N.Y. 1996; “Molecular Cell Biology”, Darnell, Lodish, Baltimore, Scientific American Books, New York, N.Y. 1986; the disclosures of all of these dictionaries and treatises are incorporated herein by reference.

### BRIEF DESCRIPTION OF THE DRAWINGS

FIG. 1. Structure comparisons of NT(8-13), an intermediate NT analogue, and ABS201.

FIG. 2. Representative Examples of Compounds of Formulas I-V.

FIG. 3A-3C. Scheme for Synthesis of Compounds of Formulas I-V.

FIG. 4. Asymmetric synthesis of ω-bromo acids.

FIG. 5. Synthesis of ethylene-bridged (N^{δ} to N^{ω}) arginine analogues.

FIG. 6. Synthesis of cyclic and acyclic arginine analogues.

FIG. 7. Peptide synthesis of representative peptides of the invention.

FIG. 8A-8C. Comparisons of induced hypothermia by α-methyl NT(8-13) analogues.

FIG. 9. Hypothermic effects of ABS201 after IP (solid symbols) and oral administration (open symbols) at 2 mg/kg and 20 mg/kg, respectively.

FIG. 10A-10B. Comparison of hypothermic effects after IP and oral administration for KH29 (10A) and KH30 (10B).

FIG. 11A-11B. Dose-response curves for ABS201 after IP administration.

FIG. 12. Dose-dependent hypothermic response to ABS201 after oral administration.

FIG. 13. Attenuation of d-amphetamine induced hyperactivity after IP administration of ABS201.

FIG. 14. Attenuation of d-amphetamine induced hyperactivity after oral administration of ABS201.

FIG. 15. Reversal of PPI induced in Brattleboro rats by ABS201 administered orally.

FIG. 16. Effect of ABS201 and haloperidol on catalepsy.

FIG. 17. Hypothermic effects of chronic administration of ABS201 after daily dose of 5 mg/kg ABS201.

FIG. 18. Effect of repeated daily administration of ABS201 on d-amphetamine induced hyperlocomotion after daily doses of 5 mg/kg ABS201.

FIG. 19. Synthesis of Fmoc-Proline-OH*.

FIG. 20. Blood concentration-time curve in male SD rats following intravenous administration of ABS201.TFA at a dose of 5 mg/kg Free Base (n=3).

FIG. 21. Blood concentration-time curve in male SD rats following oral administration of ABS201.TFA at a dose of 50 mg/kg Free Base (n=3).

FIG. 22. Plasma and brain concentration-time curve in male SD rats following intravenous administration of ABS201.TFA at a dose of 100 mg/kg free base (n=3).

FIG. 23. Comparison of the sites of absorbtion of ABS 201 in the GI tract.

FIG. 24. Antinociceptive Activity of ABS201 (5 mg/kg) and Morphine (5 mg/kg).

FIG. 25. Evaluation of ABS tolerance in rats dosed daily with 5 mg/kg ABS201 for 5 days as determined by of MPEs.

FIG. 26. The structures of NT[8-13] and peptides ABS1, ABS13, ABS201, ABS15, ABS16, ABS17 and ABS19, which exhibit enhanced NTR-2 to NTR-1 binding affinities.

FIG. 27. Structures of peptides designed to be better NTR-2 selective ligands.

FIG. 28. Analgesic effects of (A) ABS201, (B) ABS205, (C) ABS210, (D) ABS212, and (E) ABS220 administered by gavage at a dose of 20 mg/kg.

FIG. 29. Analgesic effect of (A) ABS232 and (B) ABS239 administered by gavage at a dose of 20 mg/kg.

FIG. 30. Analgesic effects of ABS212 determined using the hotplate assay. Comparison of the analgesic effects of ABS212 (10 mg/kg) and morphine (5 mg/kg) (A); dose response data for ABS212 administered by I.P. injection (B) or orally (C).

FIG. 31. Analgesic effects of ABS212 determined using the formalin assay. Comparison of the analgesic effects of ABS212 (10 mg/kg) and morphine (5 mg/kg) (A); dose response data for ABS212 administered by I.P. injection (B) or orally (C).

FIG. 32. Dose response data for ABS212 administered I.P. (A) and orally (B) as determined in the tail flick assay.

FIG. 33. Effects of ABS212 administered by I.P. injection determined using the Chung model of neuropathic pain.

FIG. 34. Concentration-Time curve in male SD rats following intravenous administration of ABS212.2HCl at a dose of 5 mg/kg free base (A) and oral administration of ABS212.2HCl at a dose of 50 mg/kg free base (B), (n=3).

### DETAILED DESCRIPTION OF THE INVENTION

The present invention is directed to certain desamino amino acid compounds, their incorporation as extenders or as congeners in known biologically active peptides, and the use of the compounds and peptides in medical diagnosis, treatment and screening. Several aspects of the invention concern the mimicry of the desamino amino acid compounds for the natural amino acids arginine and/or lysine. By their use as congeners for these natural amino acid moieties in known biologically active peptides, truncated versions of the peptides can be prepared in which the biological activity is more selective and longer lasting than that of the known peptide. By their use as extenders, their position as the N-terminal moiety adduct of a known biologically active peptide will also provide longer lasting biological activity than that of the known peptide.

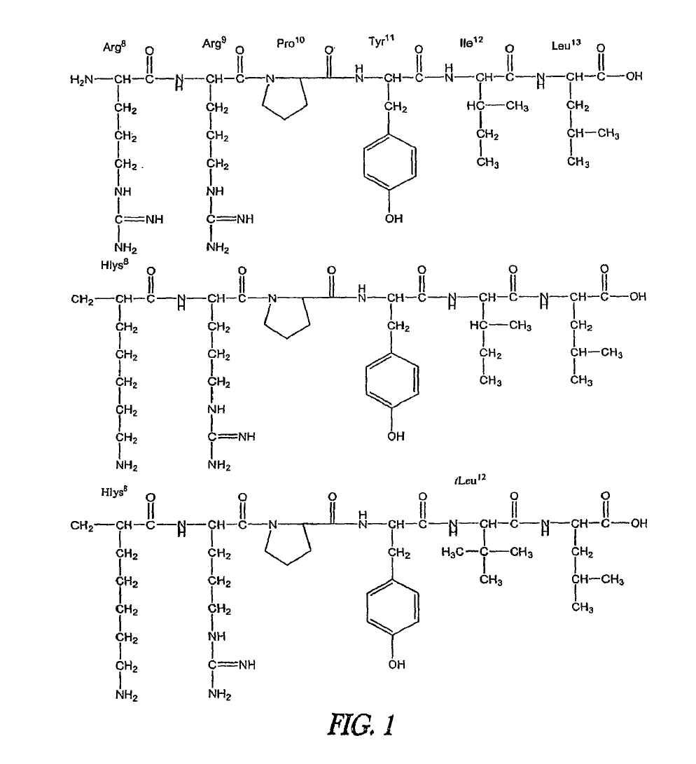

An example of the use of the desamino amino acid compounds of the invention in a truncated peptide is provided by neurotensin. Neurotensin (NT) is a 13 amino acid peptide having neurological properties. Its cleavage at AA7 to produce truncated neurotensin (8-13) provides a peptide having selective biological activity. According to the invention, conversion of the AA8 arginine to a desamino amino acid moiety results in a peptide also having significant and selective biological activity. The examples of NT and the converted versions are shown in FIG. 1.

The biologically active peptides of the present invention have the desamino amino acid moiety as their N-terminal moiety. These peptides have known amino acid sequences of biologically active amino acids wherein the desamino amino acid is either covalently coupled through an amide bond with the N-terminus amine group of the known peptide (extended peptide) or is substituted for its corresponding congener moiety (analogous natural amino acid moiety) within the peptide (truncated version). In another alternative, the peptide becomes truncated at the position of substitution so that the desamino amino acid moiety becomes the new N-terminus and the amino acid residues upstream of this position are no longer part of the sequence (truncated peptide). The extended and truncated peptides can have longer lifetimes in vivo and can have biological activities like those of the natural peptides except that the activities will be more selective.

One aspect of the desamino amino acid compounds of the invention is provided by Formula I given above. Preferred embodiments of Formula I include those wherein

R₁, R₂, and R₃ are independently, hydrogen or lower branched or straight chain alkyl of C₁-C₅, more preferably hydrogen or methyl, provided that when m is 0 or 1 and n is 0 to 5, R₁, R₂, and R₃ are not all H. In another embodiment, n is 4. In yet a further embodiment, R is an H, methyl, ethyl or propyl. Additional preferred embodiments include those wherein R is H, methyl, ethyl, propyl or butyl and:

a) n is 4, m is 0, R₁ is hydrogen, R₂ is methyl, the compound of formula I is an acid, and the stereochemistry at C_{α} is R or S if the substituent at R is methyl, ethyl, propyl or butyl;

b) n is 4, m is 1, R₁ and R₂ are methyl, R₃ is hydrogen or methyl, the compound of formula I is an acid, the stereochemistry at C_{α} is R or S if the substituent at R is methyl, ethyl, propyl or butyl;

c) n is 4, m is 1, R₁ is methyl, R₂, and R₃ are hydrogen, the compound of formula I is an acid, the stereochemistry at C_{α} is R or S if the substituent at R is methyl, ethyl, propyl or butyl;

d) n is 4, m is 1, R₁, R₂, and R₃ are hydrogen, the compound of formula I is an acid, and the stereochemistry at C_{α} is R or S if the substituent at R is methyl, ethyl, propyl or butyl;

e) n is 3, m is 0, R₁ and R₂ are methyl, the compound of formula I is an acid, the stereochemistry at C_{α} is R or S if the substituent at R is methyl, ethyl, propyl or butyl;

f) n is 3, m is 0, R₁ and R₂ are ethyl, the compound of formula I is an acid, the stereochemistry at C_{α} is R or S if the substituent at R is methyl, ethyl, propyl or butyl;

g) n is 3, m is 0, R₁ and R₂ are propyl, the compound of formula I is an acid, the stereochemistry at C_{α} is R or S if the substituent at R is methyl, ethyl, propyl or butyl;

h) n is 3, m is 0, R₁ and R₂ are butyl, the compound of formula I is an acid, the stereochemistry at C_{α} is R or S if the substituent at R is methyl, ethyl, propyl or butyl;

i) n is 2, m is 0, R₁ and R₂ are methyl, the compound of formula I is an acid, the stereochemistry at C_{α} is R or S if the substituent at R is methyl, ethyl, propyl or butyl;

j) n is 2, m is 0, R₁ and R₂ are ethyl, the compound of formula I is an acid, the stereochemistry at C_{α} is R or S if the substituent at R is methyl, ethyl, propyl or butyl;

k) n is 2, m is 0, R₁ and R₂ are propyl, the compound of formula I is an acid, the stereochemistry at C_{α} is R or S if the substituent at R is methyl, ethyl, propyl or butyl;

l) n is 2, m is 0, R₁ and R₂ are butyl, the compound of formula I is an acid, the stereochemistry at C_{α} is R or S if the substituent at R is methyl, ethyl, propyl or butyl.

Also preferred are the esters or salts of any of the foregoing preferred embodiments a-l.

Another aspect of the desamino amino acid compounds of the invention is illustrated by Formula II given above. Preferred embodiments of Formula II include those wherein when n is 3, dashed line a is not present. Additional preferred embodiments include those wherein X is hydrogen, and wherein Y and R₄ are the same lower branched or straight chain alkyl. In yet another preferred embodiment, R₄ and R₅ are, independently, hydrogen or methyl. In another preferred embodiment, dashed line a is not present, X is hydrogen or lower branched or straight chain alkyl of C₁-C₅, preferably methyl or ethyl, and Y is hydrogen or lower branched or straight chain alkyl of C₁-C₅, preferably methyl, or dashed line a is present and z is 2, and preferably, n is 3. Additional preferred embodiments include those wherein R is H, methyl, ethyl, propyl or butyl and:

a) n is 3, dashed line a is not present, the compound of formula II is an acid, R₄ is hydrogen, X is hydrogen, Y is methyl, and the stereochemistry at C_{α} is R or S if the substituent at R is methyl, ethyl, propyl or butyl;

b) n is 3, dashed line a is not present, the compound of formula II is an acid, R₄ is methyl, X is hydrogen, Y is methyl, and the stereochemistry at C_{α} is R or S if the substituent at R is methyl, ethyl, propyl or butyl;

c) n is 3, dashed line a is present, the compound of formula II is an acid, z is 2, R₄ is hydrogen, and the stereochemistry at C_{α} is R or S if the substituent at R is methyl, ethyl, propyl or butyl;

d) n is 3, dashed line a is present, the compound of formula II is an acid, z is 2, R₄ is methyl, and the stereochemistry at C_{α} is R or S if the substituent at R is methyl, ethyl, propyl or butyl;

e) n is 3, dashed line a is not present, the compound of formula II is an acid, R₄ is hydrogen, X is methyl, Y is hydrogen, and the stereochemistry at C_{α} is R or S if the substituent at R is methyl, ethyl, propyl or butyl;

f) n is 3, dashed line a is not present, the compound of formula II is an acid, R₄ is hydrogen, X is ethyl, Y is hydrogen, and the stereochemistry at C_{α} is R or S if the substituent at R is methyl, ethyl, propyl or butyl;

g) n is 2, dashed line a is not present, the compound of formula II is an acid, R₄ is hydrogen, X is hydrogen, Y is methyl, and the stereochemistry at C_{α} is R or S if the substituent at R is methyl, ethyl, propyl or butyl;

h) n is 2, dashed line a is not present, the compound of formula II is an acid, R₄ is methyl, X is hydrogen, Y is propyl, and the stereochemistry at C_{α} is R or S if the substituent at R is methyl, ethyl, propyl or butyl;

i) n is 4, dashed line a is present, the compound of formula II is an acid, z is 2, R₄ is hydrogen, and the stereochemistry at C_{α} is R or S if the substituent at R is methyl, ethyl, propyl or butyl;

j) n is 3, dashed line a is present, the compound of formula II is an acid, z is 2, R₄ is methyl, and the stereochemistry at C_{α} is R or S if the substituent at R is methyl, ethyl, propyl or butyl;

k) n is 2, dashed line a is present, the compound of formula II is an acid, z is 3, R₄ is methyl, and the stereochemistry at C_{α} is R or S if the substituent at R is methyl, ethyl, propyl or butyl;

l) n is 3, dashed line a is not present, the compound of formula II is an acid, R₄ is methyl, X is hydrogen, Y is ethyl, and the stereochemistry at C_{α} is R or S if the substituent at R is methyl, ethyl, propyl or butyl.

Also preferred are the esters or salts of any of the forgoing preferred embodiments a-l.

A third aspect of the desamino amino acid compounds of the invention is illustrated by Formula III. Preferred embodiments of Formula III include those wherein R₆ and R₇ are independently, hydrogen or lower alkyl or straight chain alkyl of C₁-C₅, preferably hydrogen or methyl, even more preferably all are hydrogen. In another embodiment, z is 2 or 3, preferably 3. In a preferred embodiment, n is 3. Additional preferred embodiments include those wherein R is H, methyl, ethyl, propyl or butyl and:

a) n is 3, z is 2, R₆ and R₇ are hydrogen, the compound of formula III is an acid, and the stereochemistry at C_{α} is R or S if the substituent at R is methyl, ethyl, propyl or butyl;

b) n is 3, z is 3, R₆ and R₇ are hydrogen, the compound of formula III is an acid, and the stereochemistry at C_{α} is R or S if the substituent at R is methyl, ethyl, propyl or butyl;

c) n is 2, z is 2, R₆ and R₇ are hydrogen, the compound of formula III is an acid, and the stereochemistry at C_{α} is R or S if the substituent at R is methyl, ethyl, propyl or butyl;

d) n is 4, z is 2, R₆ and R₇ are hydrogen, the compound of formula III is an acid, and the stereochemistry at C_{α} is R or S if the substituent at R is methyl, ethyl, propyl or butyl;

e) n is 2, z is 3, R₆ and R₇ are hydrogen, the compound of formula III is an acid, and the stereochemistry at C_{α} is R or S if the substituent at R is methyl, ethyl, propyl or butyl;

f) n is 4, z is 3, R₆ and R₇ are hydrogen, the compound of formula III is an acid, and the stereochemistry at C_{α} is R or S if the substituent at R is methyl, ethyl, propyl or butyl;

g) n is 2, z is 2, R₆ and R₇ are methyl, the compound of formula III is an acid, and the stereochemistry at C_{α} is R or S if the substituent at R is methyl, ethyl, propyl or butyl;

h) n is 4, z is 2, R₆ and R₇ are methyl, the compound of formula III is an acid, and the stereochemistry at C_{α} is R or S if the substituent at R is methyl, ethyl, propyl or butyl;

i) n is 2, z is 3, R₆ and R₇ are methyl, the compound of formula III is an acid, and the stereochemistry at C_{α} is R or S if the substituent at R is methyl, ethyl, propyl or butyl;

j) n is 4, z is 3, R₆ and R₇ are methyl, the compound of formula III is an acid, and the stereochemistry at C_{α} is R or S if the substituent at R is methyl, ethyl, propyl or butyl.

Also preferred are the esters or salts of the preferred foregoing embodiments a-j.

A fourth aspect of the invention is provided by the desamino amino acid compounds of Formula IV. Preferred embodiments of the compounds of Formula IV include those wherein R₉, R₁₀, and R₁₁ are, independently, hydrogen or lower straight or branched chain alkyl of C₁-C₅, preferably hydrogen, methyl or ethyl. In another embodiment, R₁₀ is methyl. In yet another preferred embodiment, R₉ is hydrogen, R₁₀ is methyl, R₁₁ is hydrogen, and n is 3. Additional preferred embodiments include those wherein R is H, methyl, ethyl, propyl or butyl and:

a) n is 3, R₉ and R₁₁ are hydrogen, R₁₀ is methyl, the compound of formula IV is an acid, and the stereochemistry at C_{α} is R or S if the substituent at R is methyl, ethyl, propyl or butyl;

b) n is 3, R₉ is hydrogen, R₁₀ and R₁₁ are methyl, the compound of formula IV is an acid, and the stereochemistry at C_{α} is R or S if the substituent at R is methyl, ethyl, propyl or butyl;

c) n is 3, R₉ is hydrogen, R₁₀ is methyl, R₁₁ is ethyl, the compound of formula IV is an acid, and the stereochemistry at C_{α} is R or S if the substituent at R is methyl, ethyl, propyl or butyl;

d) n is 2, R₉ and R₁₁ are hydrogen, R₁₀ is methyl, the compound of formula IV is an acid, and the stereochemistry at C_{α} is R or S if the substituent at R is methyl, ethyl, propyl or butyl;

e) n is 2, R₉ is hydrogen, R₁₀ and R₁₁ are methyl, the compound of formula IV is an acid, and the stereochemistry at C_{α} is R or S if the substituent at R is methyl, ethyl, propyl or butyl;

f) n is 4, R₉ is are hydrogen, R₁₀ is methyl, R₁₁ is ethyl, the compound of formula IV is an acid, and the stereochemistry at C_{α} is R or S if the substituent at R is methyl, ethyl, propyl or butyl.

Also preferred are the esters or salts of the foregoing preferred embodiments a-f.

A fifth aspect of the invention is provided by the desamino amino acid compounds of formula V. Preferred embodiments of the compounds of formula V include those wherein R₁₂, R₁₃, and R₁₄ are, independently, hydrogen or lower straight or branched chain alkyl of C₁-C₅, preferably hydrogen, methyl or ethyl. In another embodiment, R₁₂ is methyl. In yet another preferred embodiment, R₁₃ can be hydrogen, R₁₄ can methyl, ethyl or propyl, R₁₃ and R₁₄ can be any combination of hydrogen, methyl, ethyl or propyl, and n is 2 or 3. Additional preferred embodiments include those wherein R is H, methyl, ethyl, propyl or butyl and:

a) n is 3, R₁₂ and R₁₃ are hydrogen, R₁₄ is methyl, the compound of formula V is an acid, and the stereochemistry at C_{α} is R or S if the substituent at R is methyl, ethyl, propyl or butyl;

b) n is 3, R₁₂ is methyl, R₁₃ and R₁₄ are methyl, the compound of formula V is an acid, and the stereochemistry at C_{α} is R or S if the substituent at R is methyl, ethyl, propyl or butyl;

c) n is 3, R₁₂ is methyl, R₁₃ is methyl, R₁₄ is ethyl, the compound of formula V is an acid, and the stereochemistry at C_{α} is R or S if the substituent at R is methyl, ethyl, propyl or butyl;

d) n is 2, R₁₂ is methyl, R₁₃ is hydrogen and R₁₄ is methyl, the compound of formula V is an acid, and the stereochemistry at C_{α} is R or S if the substituent at R is methyl, ethyl, propyl or butyl;

e) n is 2, R₁₂ is methyl, R₁₃ and R₁₄ are methyl, the compound of formula V is an acid, and the stereochemistry at C_{α} is R or S if the substituent at R is methyl, ethyl, propyl or butyl;

n is 4, R₁₂ is methyl, R₁₃ is methyl, and R₁₄ is ethyl, the compound of formula V is an acid, and the stereochemistry at C_{α} is R or S if the substituent at R is methyl, ethyl, propyl or butyl.

Also preferred are the esters or salts of the foregoing preferred embodiments a-f.

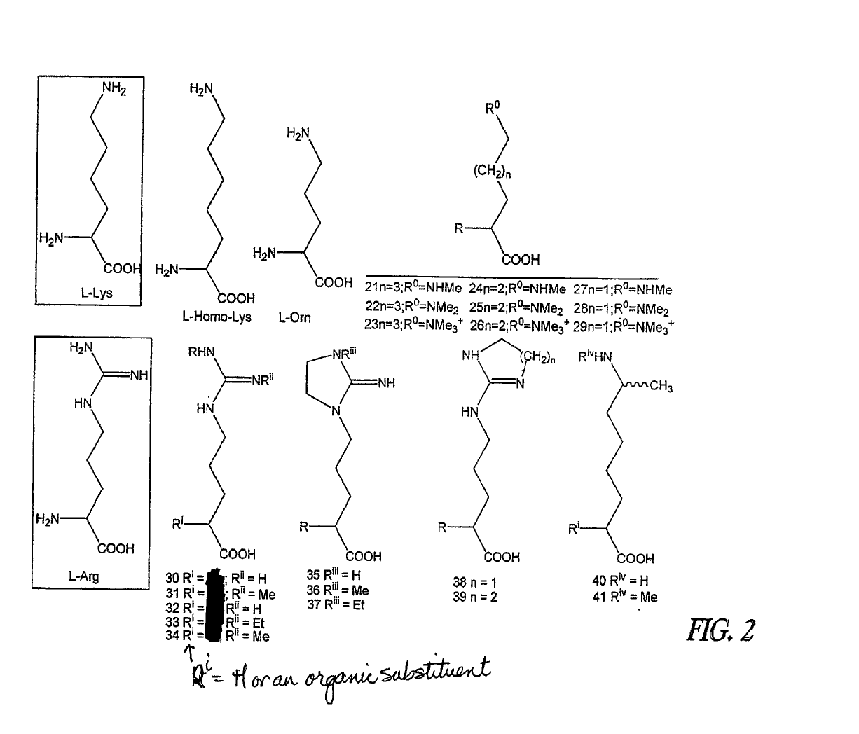

Especially preferred non-natural desamino amino acid compounds of the invention include the formulas provided in FIG. 2 wherein R is H, methyl or ethyl.

Certain embodiments of the invention provide protected intermediates and protected non-natural amino acids of the invention. Certain embodiments provide protected intermediates and protected non-natural amino acids of the invention, wherein the side chain amine group is protected by a protecting group that prevents undesired reaction of the amino group and is removable by a chemical method that does not also cause amide group cleavage. Certain embodiments provide protected intermediates and protected non-natural amino acids of the invention, wherein the side chain carboxyl group is protected by a protecting group that prevents undesired reaction of the carboxyl group and is removable by a chemical method that does not also cause carboxyl group cleavage. In certain embodiments, the protecting group is t-butoxy carbonyl (BOC) or fluorenylmethoxycarbonyl (FMOC). In certain embodiments, the protecting group is BOC, FMOC, Alloc (allyloxycarbonyl), CBZ (benzyloxycarbonyl), Pbf (2,2,4,6,7-pentamethyldihydrobenzofuran-5-sulfonyl), NO2 (nitro), Pmc (2,2,5,7,8-pentamethylchroman-6-sulfonyl), Mtr (4-methoxy-2,3,6-trimethylbenzenesulfonyl), or Tos (tosyl).

In one embodiment, the structures of the non-natural desamino amino acids of formulas I-V are similar to those of the naturally occurring amino acids lysine, arginine as well as the naturally occurring glutamate biosynthesis intermediate, ornithine. In preferred embodiments, the compounds of the invention differ from the corresponding natural amino acids due, inter alia, a longer or shorter methylene bridge between the (i) carboxyl terminus, which forms the N-terminus bond with the adjacent amino acid unit in a peptide, (ii) the presence of a —H or an alkyl group in place of the alpha amino group, and (iii) the organo group substitution of the amine side chain. Preferably, the extended bridge of the invention compared to the natural amino acid bridge is one carbon length longer or shorter (i.e., the homo- or des-forms). In other preferred embodiments, the compounds of the invention have, inter alia, longer, shorter, or equivalent methylene bridge lengths and have substitutions at various moieties, form different moieties, or link moieties to form ring structures, compared to the comparable natural amino acid.

Each of the compounds of the invention can be prepared as the acid, amide, salt or ester. In water, the non-natural amino acids of the present invention will be charged; however; in cell membranes and other non-polar regions of the cell, the non-natural amino acids may not be charged. In one embodiment, the ester group of the non-natural amino acids of the present invention is methyl, ethyl, t-butyl, benzyl or allyl. In another embodiment, the counter-ion for the salts of the non-natural amino acids is sodium, potassium, ammonium and tetra-alkyl ammonium.

Some embodiments of the invention provide semisynthetic peptides comprising a non-natural amino acid compound of the invention. In some embodiments, the semisynthetic peptide comprises a non-natural amino acid compound as its N-terminus moiety. In some embodiments, the semisynthetic peptide comprises a non-natural amino acid compound as the N-terminal moiety of a semisynthetic peptide of neurotensin (8-13). In one embodiment, the semisynthetic peptide is ABS201, ABS202, ABS205, ABS207, ABS208, ABS210, ABS211, ABS212, ABS220, ABS225, ABS226, ABS227, ABS228, ABS230, ABS232, ABS234 or ABS239. In some embodiments, the semisynthetic peptide has an extended half-life in vivo as compared to a peptide having the same sequence as the semisynthetic peptide that does not comprise the non-natural amino acid compound substituted as its N-terminus moiety. In some embodiments, the semisynthetic peptide has analgesic and/or antipsychotic activity. Examples of semisynthetic peptides having antipsychotic and/or analgesic activity are shown in the tables below.

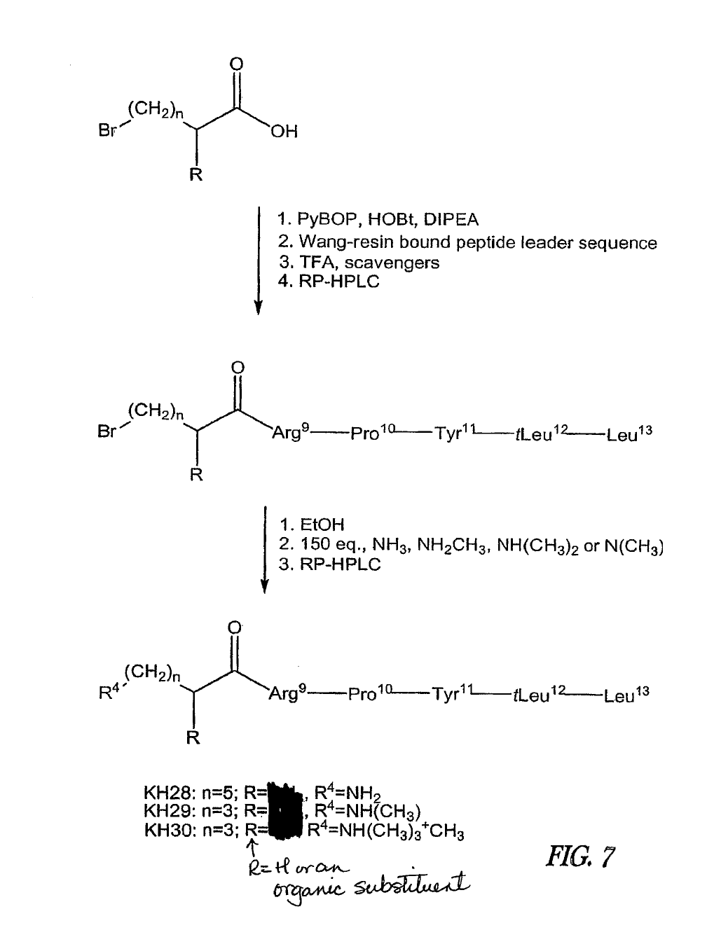

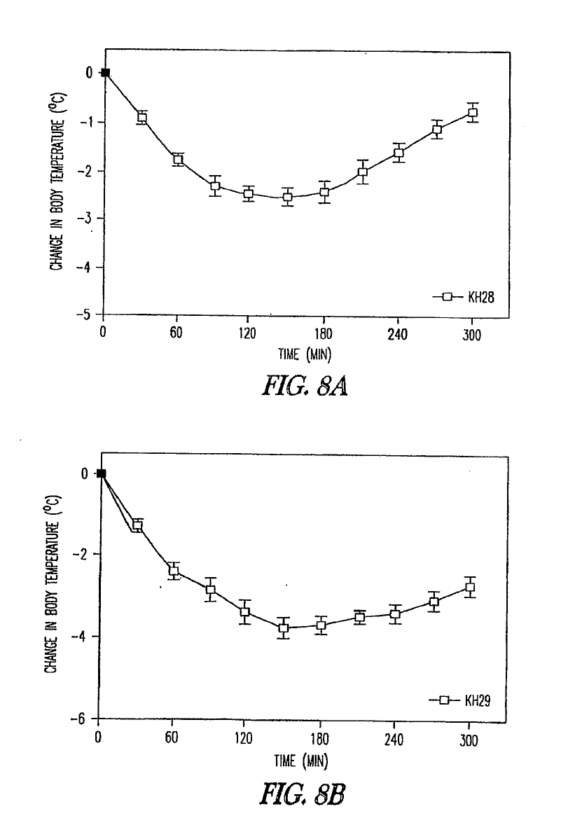

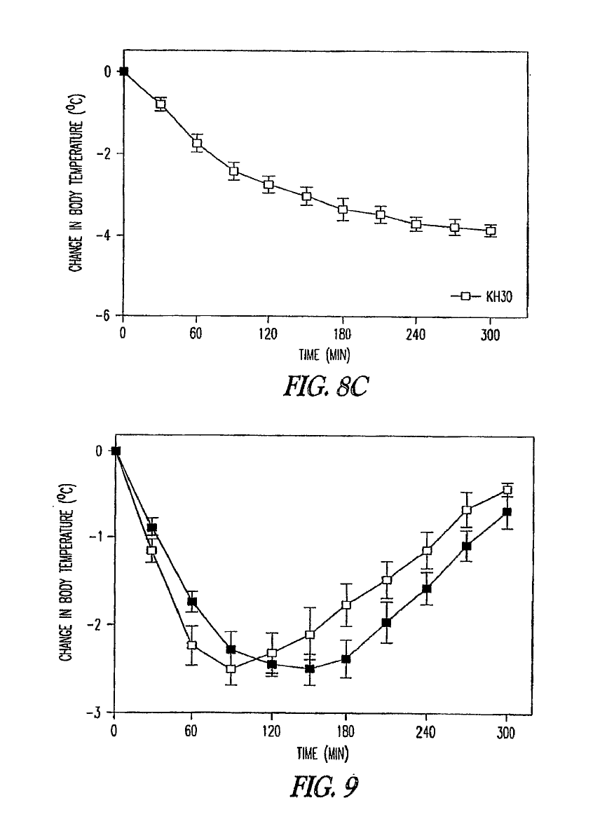

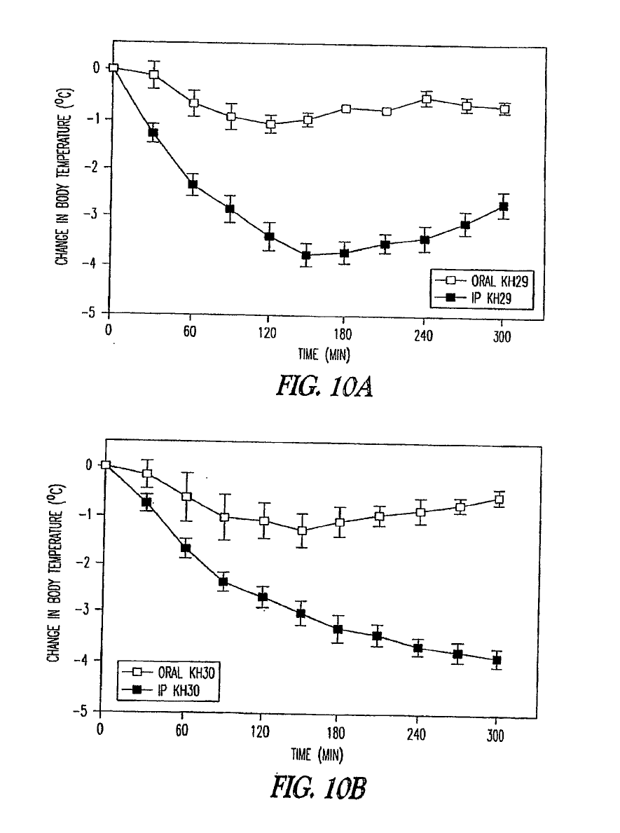

TABLE 1
Semisynthetic Peptides with Antipsychotic Property

| Peptides | N-terminus | Sequence* | Structure* |
|---|---|---|---|
| ABS201 | CH₃ | L-hLys-Arg-Pro-Tyr-tLeu-Leu | FIG. 7 |
| (KH28) | | | (KH28) |
| KH29 | CH₃ | 7-Arg-Pro-Tyr-tLeu-Leu | FIG. 7 |
| KH30 | CH₃ | 9-Arg-Pro-Tyr-tLeu-Leu | FIG. 7 |
| ABS13 | N₃ | L-hLys-Arg-Pro-Tyr-tLeu-Leu | Scheme 1 |
| ABS41 | N₃ | 13-Arg-Pro-Tyr-tLeu-Leu | Scheme 1 |
| ABS44 | N₃ | 7-Arg-Pro-Tyr-tLeu-Leu | Scheme 1 |
| ABS46 | N₃ | 9-Arg-Pro-Tyr-tLeu-Leu | Scheme 1 |
| ABS201 | CH₃ | L-hLys-Arg-Pro-Tyr-tLeu-Leu | Scheme 1 |
| ABS202 | CH₃ | 28-Arg-Pro-Tyr-tLeu-Leu | Scheme 2 |
| ABS203 | CH₃ | 29-Arg-Pro-Tyr-tLeu-Leu | Scheme 2 |

*The “tLeu” or “t-Leu” means tert-Leu. The number or term “L-hLys” in the sequence indicates a non-natural amino acid moiety, the structure of which is shown in FIG. 7 or structural Scheme 1 or 2.

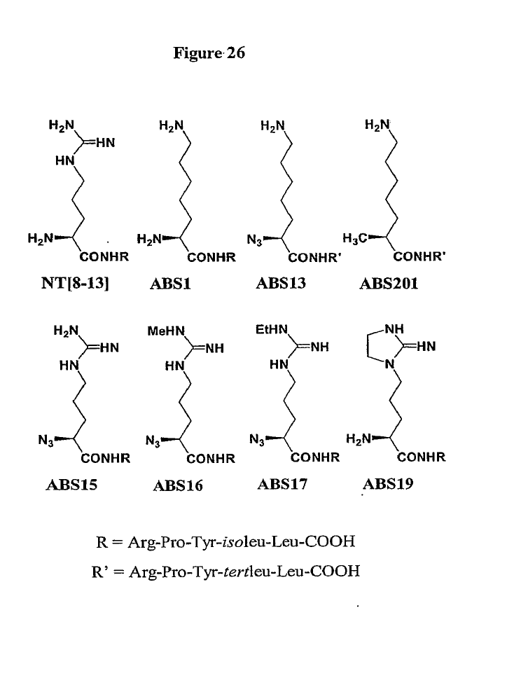

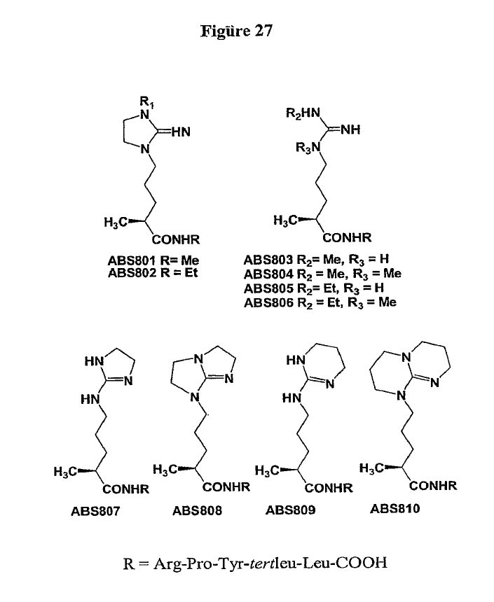

TABLE 2
Semisynthetic Peptides with Analgesic Property

| Peptide | N-terminus | Sequence* | Structure* |
|---|---|---|---|
| ABS1 | NH₂ | X-Arg-Pro-Tyr-isoleu-Leu | FIG. 26 |
| ABS13 | N₃ | X-Arg-Pro-Tyr-tleu-Leu | FIG. 26 |
| ABS201 | CH₃ | X-Arg-Pro-Tyr-tleu-Leu | FIG. 26 |
| ABS15 | N₃ | X-Arg-Pro-Tyr-isoleu-Leu | FIG. 26 |
| ABS16 | N₃ | X-Arg-Pro-Tyr-isoleu-Leu | FIG. 26 |
| ABS17 | N₃ | X-Arg-Pro-Tyr-isoleu-Leu | FIG. 26 |
| ABS19 | NH₂ | X-Arg-Pro-Tyr-isoleu-Leu | FIG. 26 |

*The letter “X” in the sequence indicates a non-natural amino acid moiety, the structure of which is shown in FIG. 26.

TABLE 3
Semisynthetic Peptides with Analgesic and Antipsychotic
Property

| Peptides | N-terminus | Sequence* | Structure* |
|---|---|---|---|
| ABS201 | CH₃ | hLys-Arg-Pro-Tyr-t-Leu-Leu | Scheme 3 |
| ABS202 | CH₃ | 1-Arg-Pro-Tyr-t-Leu-Leu | Scheme 3 |
| ABS203 | CH₃ | 2-Arg-Pro-Tyr-t-Leu-Leu | Scheme 3 |
| ABS204 | CH₃ | 3-Arg-Pro-Tyr-t-Leu-Leu | Scheme 3 |
| ABS205 | CH₃ | Lys-Arg-Pro-Tyr-t-Leu-Leu | Scheme 3 |
| ABS206 | CH₃ | 4-Arg-Pro-Tyr-t-Leu-Leu | Scheme 3 |
| ABS207 | CH₃ | 5-Arg-Pro-Tyr-t-Leu-Leu | Scheme 3 |
| ABS208 | CH₃ | 6-Arg-Pro-Tyr-t-Leu-Leu | Scheme 3 |
| ABS209 | CH₃ | Orn-Arg-Pro-Tyr-t-Leu-Leu | Scheme 3 |
| ABS210 | CH₃ | 7-Arg-Pro-Tyr-t-Leu-Leu | Scheme 3 |
| ABS211 | CH₃ | 8-Arg-Pro-Tyr-t-Leu-Leu | Scheme 3 |
| ABS212 | CH₃ | 9-Arg-Pro-Tyr-t-Leu-Leu | Scheme 3 |
| ABS213 | CH₃ | Arg-Arg-Pro-Tyr-t-Leu-Leu | Scheme 3 |
| ABS214 | CH₃ | 10-Arg-Pro-Tyr-t-Leu-Leu | Scheme 3 |
| ABS215 | CH₃ | 11-Arg-Pro-Tyr-t-Leu-Leu | Scheme 3 |
| ABS216 | CH₃ | 12-Arg-Pro-Tyr-t-Leu-Leu | Scheme 3 |
| ABS217 | CH₃ | 13-Arg-Pro-Tyr-t-Leu-Leu | Scheme 3 |
| ABS218 | CH₃ | 18-Arg-Pro-Tyr-t-Leu-Leu | Scheme 3 |
| ABS220 | CH₃ | 15-Arg-Pro-Tyr-t-Leu-Leu | Scheme 3 |
| ABS221 | CH₃ | 16-Arg-Pro-Tyr-t-Leu-Leu | Scheme 3 |
| ABS224 | CH₃ | alkene-Arg-Pro-Tyr-t-Leu-Leu | Scheme 3 |
| ABS700 | | Boc-Arg-Pro-Tyr-t-Leu-Leu | Scheme 3 |
| ABS225 | H | hLys-Arg-Pro-Tyr-tLeu-Leu | Scheme 3 |
| ABS226 | H | 1-Arg-Pro-Tyr-tLeu-Leu | Scheme 3 |
| ABS227 | H | 2-Arg-Pro-Tyr-tLeu-Leu | Scheme 3 |
| ABS228 | H | 3-Arg-Pro-Tyr-tLeu-Leu | Scheme 3 |
| ABS229 | H | Lys-Arg-Pro-Tyr-tLeu-Leu | Scheme 3 |
| ABS230 | H | 4-Arg-Pro-Tyr-tLeu-Leu | Scheme 3 |
| ABS231 | H | 5-Arg-Pro-Tyr-tLeu-Leu | Scheme 3 |
| ABS232 | H | 6-Arg-Pro-Tyr-tLeu-Leu | Scheme 3 |
| ABS233 | H | Orn-Arg-Pro-Tyr-tLeu-Leu | Scheme 3 |
| ABS234 | H | 7-Arg-Pro-Tyr-tLeu-Leu | Scheme 3 |
| ABS235 | H | 8-Arg-Pro-Tyr-tLeu-Leu | Scheme 3 |
| ABS236 | H | 9-Arg-Pro-Tyr-tLeu-Leu | Scheme 3 |
| ABS237 | H | Arg-Arg-Pro-Tyr-tLeu-Leu | Scheme 3 |
| ABS238 | H | 10-Arg-Pro-Tyr-tLeu-Leu | Scheme 3 |
| ABS239 | H | 11-Arg-Pro-Tyr-tLeu-Leu | Scheme 3 |
| ABS240 | H | 12-Arg-Pro-Tyr-tLeu-Leu | Scheme 3 |
| ABS241 | H | 13-Arg-Pro-Tyr-tLeu-Leu | Scheme 3 |
| ABS242 | H | 18-Arg-Pro-Tyr-tLeu-Leu | Scheme 3 |
| ABS243 | H | 19-Arg-Pro-Tyr-tLeu-Leu | Scheme 3 |
| ABS244 | H | 15-Arg-Pro-Tyr-tLeu-Leu | Scheme 3 |
| ABS245 | H | 16-Arg-Pro-Tyr-tLeu-Leu | Scheme 3 |
| ABS246 | H | 17-Arg-Pro-Tyr-tLeu-Leu | Scheme 3 |

*The “tLeu” or “t-Leu” means tert-Leu. The number or term “L-hLys” in the sequence indicates a non-natural amino acid moiety, the structure of which is shown in Scheme 3.

Certain embodiments of the present invention provide pharmaceutical compositions comprising a peptide of the invention and a pharmaceutical carrier. In certain embodiments, the peptide is present in unit dosage form.

Certain embodiments of the present invention provide cosmetic formulations comprising a non-natural amino acid compound of the invention and a cosmetic base formulation. Certain embodiments of the present invention provide cosmetic formulations comprising a semisynthetic peptide of the invention and a cosmetic base formulation. In certain embodiments, the cosmetic base formulation is an aqueous or oil base.

Certain embodiments of the present invention provide the non-natural amino acid compounds of the invention for use in medical therapy.

Certain embodiments of the present invention provide the use of the non-natural amino acid compounds of the invention for the manufacture of a medicament useful for treating psychosis in a mammal. Certain embodiments of the present invention provide the use of the semisynthetic peptides of the invention for the manufacture of a medicament useful for treating psychosis in a mammal. In certain embodiments, the psychosis is schizophrenia.

Certain embodiments of the present invention provide the use of a compound of the invention for the manufacture of a medicament useful for treating cancer, obesity, Parkingson's disease or psychostimulatn abuse in a mammal.

Certain embodiments of the present invention provide the use of a compound of the invention for the manufacture of a medicament useful for treating pain in a mammal.

Certain embodiments of the present invention provide the semisynthetic peptides of the invention for use in medical therapy.

Certain embodiments of the present invention provide a method to lower the body temperature of a patient, comprising administering to the patient an effective amount of a semisynthetic peptide of the invention so as to lower the body temperature of the patient.

Certain embodiments of the present invention provide a method to lower the body temperature of a patient, comprising administering to the patient an effective amount of a composition of the invention so as to lower the body temperature of the patient.

Certain embodiments of the present invention provide a method to treat a patient with psychosis, comprising administering to the patient an effective amount of a peptide of the invention so as to treat the psychosis.

Certain embodiments of the present invention provide a method to treat a patient with psychosis, comprising administering to the patient an effective amount of a composition of the invention so as to treat the psychosis.

Certain embodiments of the present invention provide a method to treat cancer, comprising administering to a patient an effective amount of a peptide of any of the invention so as to treat the cancer.

Certain embodiments of the present invention provide a method to treat cancer, comprising administering to a patient an effective amount of a composition of the invention so as to treat the cancer.

Certain embodiments of the present invention provide a method to treat pain, comprising administering to a patient an effective amount of a peptide of the invention so as to treat the pain.

Certain embodiments of the present invention provide a method to treat pain, comprising administering to a patient an effective amount of a composition of the invention so as to treat the pain.

Certain embodiments of the present invention provide a method for screening a peptide containing a non-natural amino acid compound for an activity, comprising the steps of: a) measuring a biological activity of a first peptide having a known amino acid sequence; and b) measuring the same biological activity of a semisynthetic peptide of any of the invention wherein the semisynthetic peptide has the same sequence as the first peptide except for the non-natural amino acid compound, or is a truncated version of the first peptide except for the non-natural amino acid compound. In certain embodiments of the invention, the biological activity is poptosis, apoptosis, cell signaling, ligand binding, transcription, translation, metabolism, cell growth, cell differentiation, homeostasis, half-life, solubility, or stability. In certain embodiments of the invention, the biological activity includes a direct or indirect assessment of the ability of the semisynthetic peptide to pass through a biological barrier. In certain embodiments of the invention, the biological activity is selectivity.

Certain embodiments of the present invention provide a method of treating a patient with a disease that is affected by administration to the patient of a known first peptide, comprising administering to the patient a semisynthetic peptide of the invention wherein the semisynthetic peptide has the same sequence as the first peptide except for the non-natural amino acid compound, or is a truncated version of the first peptide except for the non-natural amino acid compound.

Certain embodiments of the present invention provide a method of increasing the ability of a known first peptide to cross a biological barrier of a subject, comprising substituting a semisynthetic peptide of the invention wherein the semisynthetic peptide has the same sequence as the first peptide except for the non-natural amino acid compound, or is a truncated version of the first peptide except for the non-natural amino acid compound. In certain embodiments, the barrier comprises the blood brain barrier, a cell membrane, intestinal epithelium, skin, or blood-ocular.

Certain embodiments of the present invention provide a method of increasing the selectivity of a known peptide, comprising substituting for the known peptide a semisynthetic peptide of the invention wherein the semisynthetic peptide has the same sequence as the first peptide except for the non-natural amino acid compound, or is a truncated version of the first peptide except for the non-natural amino acid compound.

Certain embodiments of the present invention provide a method of increasing the resistance of a known peptide to digestion by a peptidase, comprising substituting for the known peptide a semisynthetic peptide of the invention wherein the semisynthetic peptide has the same sequence as the first peptide except for the non-natural amino acid compound, or is a truncated version of the first peptide except for the non-natural amino acid compound.

Certain embodiments of the present invention provide a method of treating a patient with a disease that is affected by administration to the patient of a known first peptide that crosses a body barrier, comprising administering to the patient a semisynthetic peptide of the invention wherein the semisynthetic peptide has the same sequence as the first peptide except for the non-natural amino acid compound, or is a truncated version of the first peptide except for the non-natural amino acid compound.

Certain embodiments of the present invention provide a method of treating a patient with a disease of the brain that is affected by administration to the patient of a known first peptide, comprising administering to the patient a semisynthetic peptide of any of the invention wherein the semisynthetic peptide has the same sequence as the first peptide except for the non-natural amino acid compound, or is a truncated version of the first peptide except for the non-natural amino acid compound.

Certain embodiments of the present invention provide a method for preparing a semisynthetic peptide with an extended half-life in vivo comprising substituting for a known peptide a semisynthetic peptide of the invention wherein the semisynthetic peptide has the same sequence as the first peptide except for the non-natural amino acid compound, or is a truncated version of the first peptide except for the non-natural amino acid compound.

The compound designators ABS201, ABS48, KH48, and peptide 28, as used herein, unless otherwise indicated, represent the same compound.

### Preparation of the Desamino Amino Acid Compounds

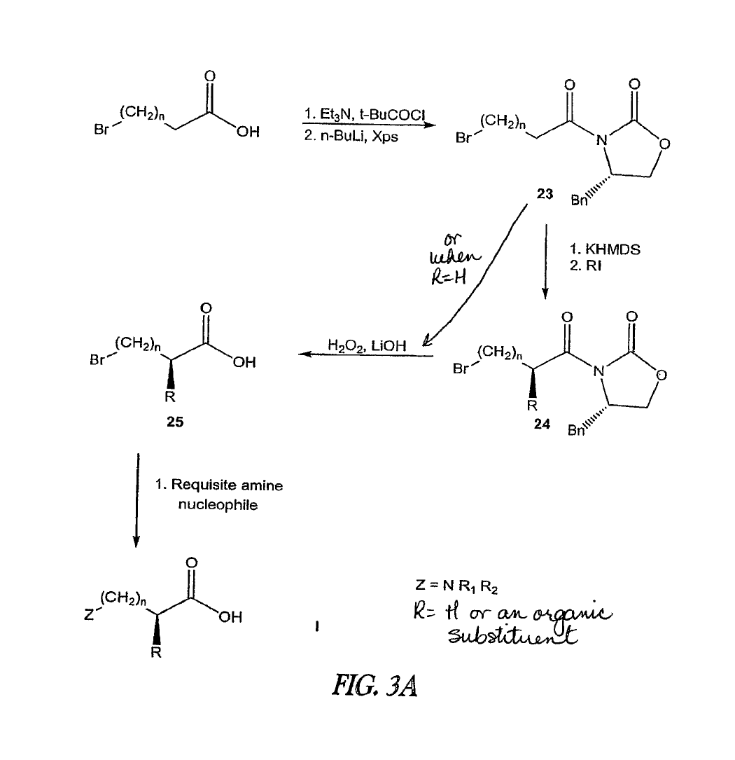

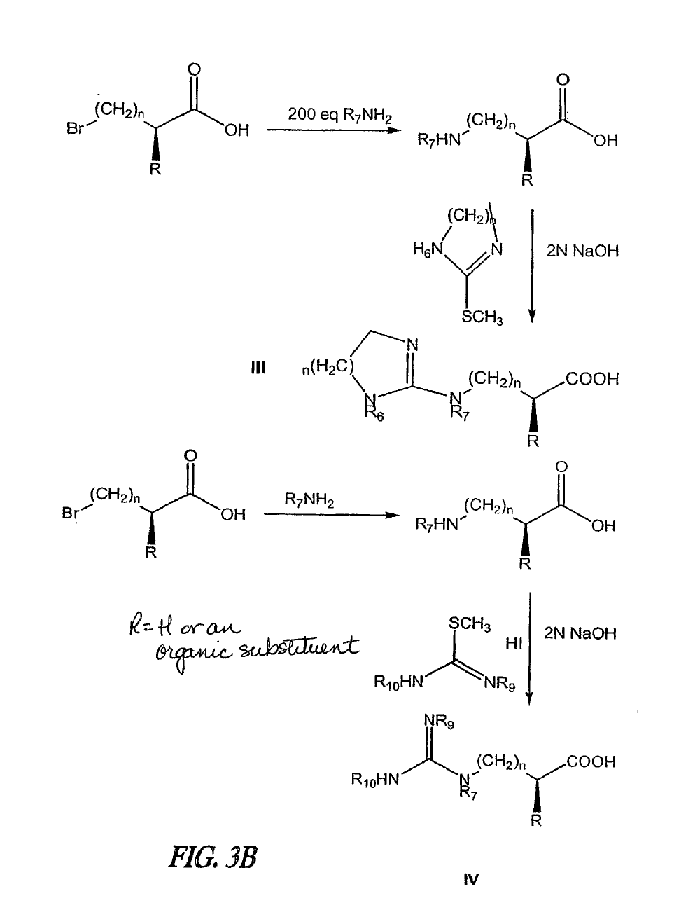

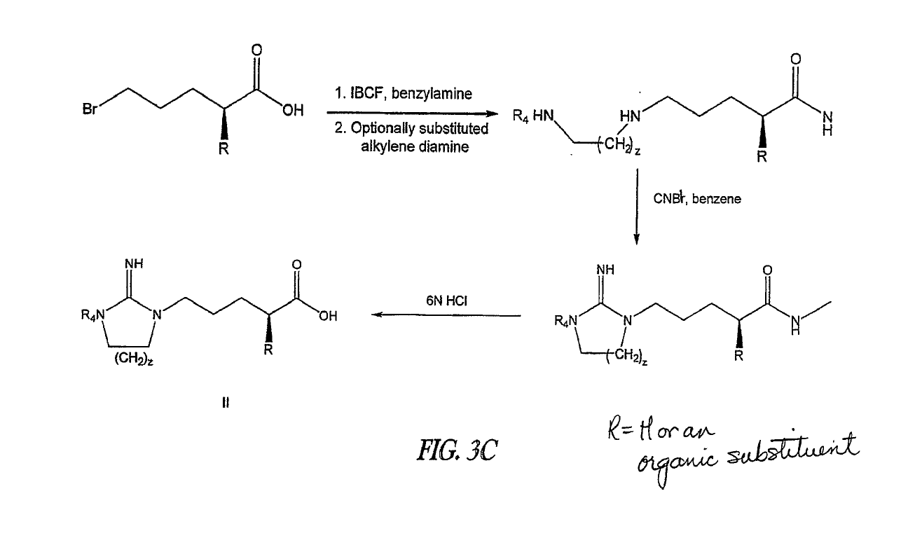

The preparation of the desamino amino acid compounds of the invention follows the overall synthetic scheme depicted in FIG. 3. The first step in this process is the production of omega halogen carboxylic acids having a methylene unit chain length corresponding to n of formulas I through V. In the following discussion, and in FIG. 3, this intermediate is designated as compound 27. Following the production of compound 27, its ω-halo group can be easily displaced with excess nucleophilic agent to produce the desamino amino acid compounds of Formulas I-V.

When R=H, the omega halogen compound can be used directly in succeeding steps. When R is an organic substituent, the R group is added through the following synthesis. When R is an organic substituent, the reaction conditions for production of compound 27 involve protection of the carboxyl group of an omega carboxylic acid by formation of an acyl oxazolone. The acyl oxazolone is converted to an enolate and the enolate is combined with an alkylating agent such as alkyl iodide or alkyl mesylate to form compound 27. Use of a large excess of the alkylating agent and long reaction times promote significant yields of compound 27.

As shown in the synthetic scheme of FIG. 3, the omega halo carboxylic acid compounds 25 can be converted to any of the side chain modifications by coupling the appropriate side chain moiety and the omega halo group of compound 25. Appropriate protection of the carboxyl group is also advantageously employed. The conditions for these reactions, and the appropriate alkylating and substituting agents follow the teaching set forth in “Advanced Organic Chemistry”, 4^{th} Edition, J. March, Wiley InterScience, New York, N.Y. 1992, the entire disclosure of which is incorporated herein by reference.

In particular, to prepare the compounds of Formula I, (see synthetic schemes of FIGS. 3 and 4), the omega halo carboxylic acid compound 27 can be combined with the appropriate amine nucleophile such as ammonia, a primary amine or a secondary amine. The formulas of the amine nucleophiles correspond to the side chain moiety of Formula I. The reaction conditions will follow those appropriate for amine nucleophilic substitution as are disclosed in “Advanced Organic Chemistry” cited above and incorporated herein as if fully repeated. These compounds can be used directly in the following peptide synthesis provided that the side chain amine group is appropriately protected or otherwise inhibited from carboxyl condensation.

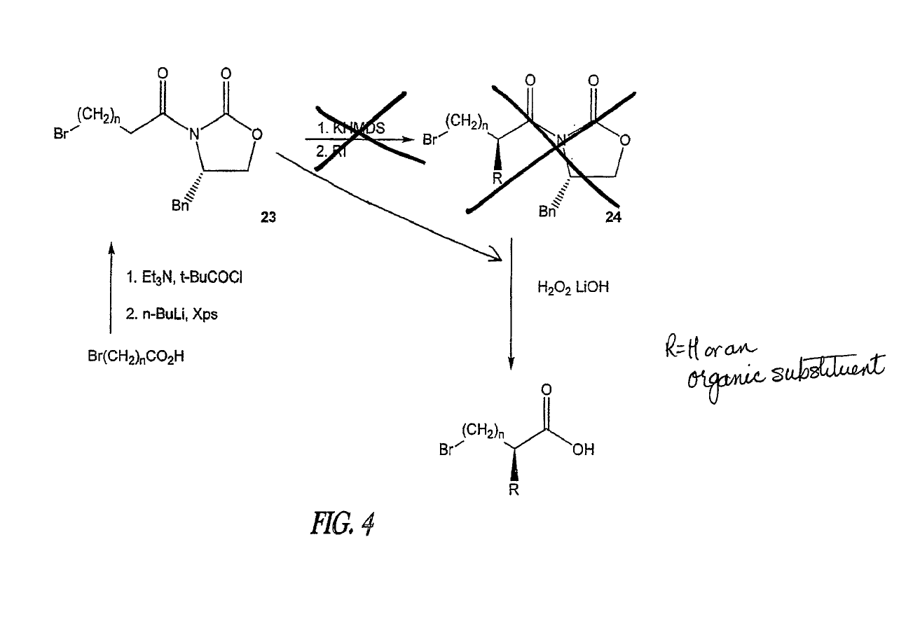

Similarly, to prepare the compounds of Formula II (see the synthetic schemes of FIGS. 3 and 5) the omega halo compound 27 may first be protected at the carboxyl position and then can be reacted sequentially with a diamine and cyanogen bromide. Deprotection and purification will afford the desamino amino acid compounds of Formula II. These compounds can be used directly in peptide synthesis with appropriate side chain protection.

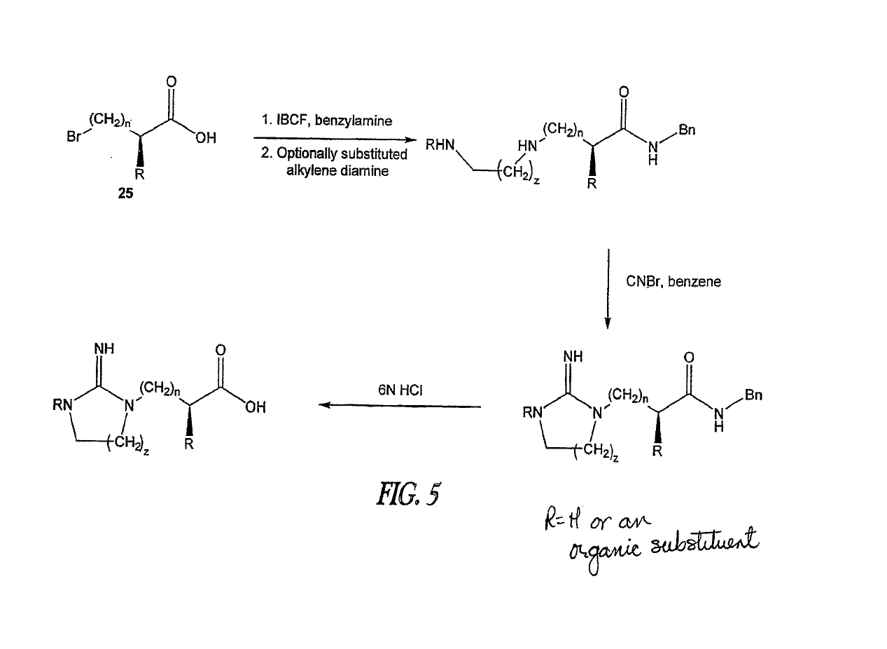

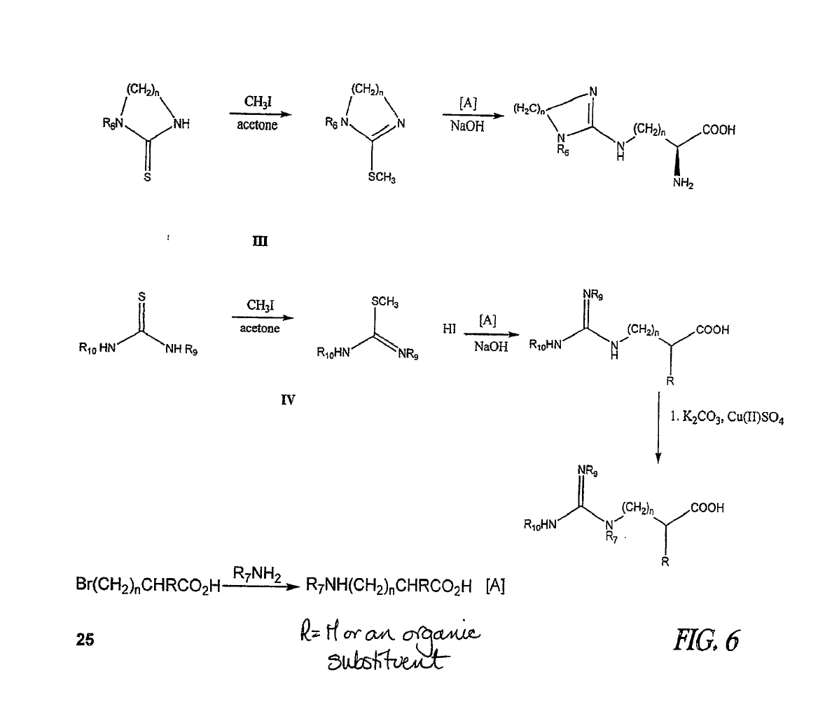

The compounds of Formulas III and IV (see the synthetic schemes of FIGS. 3 and 6) can also be prepared by addition of the side chain moiety to the omega halo carboxylic acid compounds 27. In this instance, protection of the carboxyl group is unnecessary. Preparation of the appropriate thiourea compound can be accomplished by addition of an alkylating agent such as an alkyl, alkenyl or alkynyl halide to thiourea, N-substituted thiourea or N,N-disubstituted thiourea (commercially available). The nucleophilic substitution of the resulting appropriate thiourea compound at the omega halo position of compound 27 under basic conditions provides the desamino amino acid compounds of Formula IV. Similarly, addition of the appropriate cyclic thiourea compounds to the omega halo compound 27 under basic conditions provides the desamino amino acid compounds of Formula III. The appropriate cyclic thiourea compounds can be prepared by combining an alkylating agent such as an alkyl, alkenyl or alkynyl halide with the corresponding unsubstituted, N-substituted or N,N-disubstituted cyclic diazathione (commercially available). The crude reaction products can be purified by known methods such as ion-exchange chromatography to yield the desamino amino acid compounds of Formulas III and IV which can be used directly in peptide synthesis with appropriate side chain protection.

The compounds of formula V can be prepared by first protection of the carboxyl group as an acyl oxazolone. Subsequent to formation of the protected carboxylic acid, an organic substituent at the alpha carbon (R) can be added if desired following the procedure given above. In the next step, the omega halo group can be reacted with cyanide to form an omega cyano protected carboxylic acid. This intermediate can be reacted with an alkali metal organo amine (XNR₁₃R₁₄) to form the amidine anion. The amidine anion may be quenched with acid, water or an alkyl halide to form either the unsubstituted imine moiety of the amidine or the alkyl substituted imine moiety of the amidine, respectively. This amidine protected carboxylic acid may then be employed in the synthetic scheme described above for production of the desired peptide.

Before their use in peptide synthesis, the side chains of the compounds of Formulas I-V may either be appropriately protected or determined to be sufficiently hindered that they will not enter into a peptide condensation reaction. For example, if the side chain of the compounds of Formula I is a primary amine group, it may be appropriately protected according to the teaching of the art associated with peptide synthesis. See for example, the review of amine protecting groups provided in “Compendium of Organic Synthetic Methods,” I&S Harrison, Wiley Interscience, New York, N.Y., 1971, the disclosure of which is incorporated herein by reference. In these instances, an appropriate protecting group can be t-butoxy carbonyl having the acronym BOC or fluorenylmethoxycarbonyl having the acronym FMOC. The BOC and FMOC protecting groups can be removed by mild treatment with acid, such as aqueous trifluoroacetic acid, and base, such as piperidine, respectively.

Alternatively, the omega halo carboxylic acid compound 27 can be coupled with the penultimate peptide to form an omega halo acyl moiety at the N-terminus of the penultimate peptide. Because the omega halo carboxylic acid compound 27 does not contain an amino moiety on its side chain, protection and spurious peptide formation are of less concern. In this alternative, the amino moiety can undergo nucleophilic reaction with the omega halo group of the acylated penultimate peptide as described above for formation of the compounds of Formulas I-V. The desired peptide having a residue of a compound of Formula I-V at its N-terminus is produced. In this alternative, appropriate protection of carboxyl and amino side chains and appropriate protection of the C-terminus may be employed to prevent undesirable reactions of these groups.

### Peptide Synthesis and Purification

The invention includes the truncated and extended peptides which contain as their N-terminus moiety the residue of the compound of Formula I, II, III, IV or V. These peptides can be synthesized by the Merrifield solid phase method, which is an established method for preparing peptides to those skilled in the art. See R. B. Merrifield, Science, 232, 341-347 (1986), the disclosure of which is incorporated herein by reference for an explanation of, and conditions for the Merrifield solid phase peptide synthesis. Alternatively, the peptide minus the N-terminal amino acid unit, or penultimate peptide, can be expressed recombinantly by known biological methods and the desamino amino acid compounds of Formula I-V can be added as the N-terminus by enzymatic condensation using an aminopeptidase. See “Enzyme Structure and Mechanism,” Alan Fersht, W.H. Freeman, New York, N.Y. (1985), the disclosure of which is incorporated herein by reference, for an explanation of, and conditions for, recombinant expression of peptides. The Formulas I-V compounds can be appropriately protected at the side chain amino group with standard protecting groups. In a preferred embodiment, the protecting groups are BOC and/or FMOC.

Briefly, for a solid phase synthesis, the penultimate peptide can be produced in bulk and then coupled to any of the Formula I-V compounds using the protection and coupling techniques of the Merrifield solid phase synthesis. Starting with an appropriate anchor resin designed for amino group exposure, the carboxy terminus amino acid unit of the peptide having an amino protecting group such as an FMOC group is anchored to the resin through a selectively cleavable carboxyl coupling link. The amino group of the anchored carboxy terminus unit is then deprotected and the additional amino protected amino acid units are then sequentially coupled in proper sequence. Each coupling step will involve deprotection of the protected amino group of the anchored peptide chain followed by peptide condensation between that unprotected amino group and the carboxyl group of the next amino acid unit. The condensation can be facilely obtained by carbodiimide coupling, by Schotten Bauman reaction or by activated acyl group condensation. These condensation reactions are described in “Advanced Organic Chemistry”, cited above. Protection of amine and carboxyl side chains using appropriate protecting groups that differ from the protecting groups of the alpha amino group entering into the peptide condensation will enable selective peptide condensation of the sequential amino acid units. Selection of appropriate protection groups and conditions for solid phase peptide synthesis are described in the Merrifield reference, cited above.

The penultimate peptides may also be produced by recombinant expression. This biological method involves re-engineering a microbe to express the penultimate peptide. A DNA segment encoding the penultimate peptide sequence can be inserted in proper reading from into a plasmid or other vector capable of causing microbial expression of the DNA. The vector will also contain appropriate control, promoter and selection DNA segments. Upon insertion into a microbe such as E. coli or B. subtilus, the microbe mixture can be selected for appropriate transfection by treatment with the corresponding selection agent. Typically the agent will be an antibiotic and the vector will contain a sequence encoding the corresponding detoxifying enzyme for the antibiotic. Chloramphenacol and penicillin are two of such agents. Culturing the transfected microbe and harvesting the expressed peptide as either secreted material of the culture medium or by lysing the microbe cells will provide the crude penultimate peptide. The penultimate peptide may be purified by known techniques such as lyophilization, chromatography and the like. These recombinant techniques for peptide expression are fully set forth in “Cold Spring Harbor—Current Protocols in Molecular Biology,” Wiley Interscience, Cold Spring Harbor (2003), the disclosure of which is incorporated herein by reference.

An example of the solid phase peptide production is provided by the development of a set of neurotensin (8-13) compounds (NT peptide). These compounds incorporate the desamino amino acid compounds of Formulas I-V as their N-termini. They are a novel class of antipsychotic drugs, the biological study and background of which are described in the sections below.

### NT Peptide Synthesis

### General

The penultimate sequence of the peptide, NT(9-13), can be synthesized in bulk using p-alkoxybenzyl alcohol solid phase methodology (65) and stored in the fully protected form.

Starting Materials. P-alkoxybenzyl alcohol resin-bound Nα-Fmoc-leucine, Nα-Fmoc-isoleucine, Nα-Fmoc-tert-leucine, Nα-Fmoc-(But)-tyrosine, Nα-Fmoc-(Boc)-tryptophan, Nα-Fmoc-proline, and Nα-Fmoc-(Pbf)-arginine were purchased from Advanced Chemtech (Louisville, Ky.). PyBOPO was purchased from Novabiochem (San Diego, Calif.). N-hydroxybenzoriazole (HOBt), anhydrous N,N-dimethylformamide (DMF), N,N-diisopropylethylamine (DIPEA), triisopropylsilane (TIS), and trifluoroacetic acid (TFA) were purchased from Aldrich (Milwaukee, Wis.). Non-natural amino acid analogues were used as prepared. Abbreviations. Fmoc, fluorenylmethoxycarbonyl; NH₃, ammonia; NH₂CH₃, methylamine; NH(CH₃)₂, dimethylamine; N(CH₃)₃, trimethylamine; EtOH, ethanol.

Briefly, resin-bound Nα-Fmoc-leucine can be swelled in DMF prior to Fmoc cleavage with piperidine (20% in DMF). The piperidine solution can be removed with vacuum filtration and the resin-bound amino acid washed with DMF and CH₂Cl₂. Amino acids (4 eq) can be activated in DMF with HOBt (4 eq), PyBOP (4 eq), and DIPEA (10 eq) and added directly to the peptide reaction vessel. The amino acid couplings can be conducted for approximately 6 hr, the resin washed with DMF and CH₂Cl₂ and monitored with the Kaiser test (66) for the presence of free amines. Residues can be recoupled when necessary.

This procedure was repeated with subsequent amino acids to give the penultimate peptide sequence. Aliquots of the resin-bound pentamer can then be coupled as described above with the Formula I-V compounds to give the desired peptides. Acid-catalyzed deprotection can be performed with a TFA solution containing appropriate scavengers and crude peptides can be precipitated in ice-cold ether.

### Peptide Purification

### General

Reverse phase high pressure liquid chromatography can be used to purify the foregoing crude peptides. For example, a Waters dual pump system in combination with a Waters C18 radial compression column can be used for this purpose. Effluent can be monitored by UV absorbance at 280 nm.

### Screening of Non-Natural Amino Acid-Containing Peptides

The invention provides a method for screening a peptide for an activity or pharmacological activity. The method includes the steps of: a) measuring an activity or pharmacological activity of a peptide having a selected natural amino acid sequence, and b) measuring the same activity or pharmacological activity of an extended or truncated peptide based upon the same amino acid sequence as the foregoing peptide wherein the N-terminus is a non-natural amino acid having the formula I-V, described above; and c) comparing the measured activity or pharmacological activity of the peptides from steps a) and b) to determine whether the peptide of step b) has the desired activity or pharmacological activity.

The activities for which the present invention screens can include any activity associated with a biologically active peptide or peptidomimetic. The following is a partial list of the many activities that can be determined in the present screening method:

1. Receptor agonist/antagonist activity: A compendia of examples of specific screens for measuring these activities can be found in: “The RBI Handbook of Receptor Classification and Signal Transduction” K. J. Watling, J. W. Kebebian, J. L. Neumeyer, eds. Research Biochemicals International, Natick, Mass., 1995, and references therein. Methods of analysis can be found in: T. Kenakin “Pharmacologic Analysis of Drug-Receptor Interactions” 2^{nd} Ed. Raven Press, New York, 1993, and references therein.

2. Enzyme inhibition: A compendia of examples of specific screens for measuring these activities can be found in: H. Zollner “Handbook of Enzyme Inhibitors”, 2^{nd} Ed. VCH Weinheim, FRG, 1989, and references therein.

3. Central nervous system, autonomic nervous system (cardiovascular and gastrointestinal tract), antihistaminic, anti-inflammatory, anaesthetic, cytotoxic, and antifertility activities: A compendia of examples of specific screens for measuring these activities can be found in: E. B. Thompson, “Drug Bioscreening: Drug Evaluation Techniques in Pharmacology”, VCH Publishers, New York, 1990, and references therein.

4. Anticancer activities: A compendia of examples of specific screens for measuring these activities can be found in: I. J. Fidler and R. J. White “Design of Models for Testing Cancer Therapeutic Agents”, Van Nostrand Reinhold Company, New York, 1982, and references therein.

5. Antibiotic and antiviral (especially anti-HIV) activities: A compendia of examples of specific screens for measuring these activities can be found in: “antibiotics in Laboratory Medicine”, 3^{rd} Ed., V. Lorian, ed. Williams and Wilkens, Baltimore, 1991, and references therein. A compendia of anti-HIV screens for measuring these activities can be found in: “HIV Volume 2: Biochemistry, Molecular Biology and Drug Discovery”, J. Karn, ed., IRL Press, Oxford, 1995, and references therein.

6. Immunomodulatory activity: A compendia of examples of specific screens for measuring these activities can be found in: V. St. Georgiev (1990) “Immunomodulatory Activity of Small Peptides” Trends Pharm. Sci. 11, 373-378.

7. Pharmacokinetic properties: The pharmacological activities assayed in the screening method include half-life, solubility, or stability, among others. For example, methods of analysis and measurement of pharmacokinetic properties can be found in: J.-P. Labaune “Handbook of Pharmacokinetics: Toxicity Assessment of Chemicals”, Ellis Horwood Ltd., Chichester, 1989, and references therein.

In the screening method, the peptide of step a) can consist of natural amino acids. Alternatively, the peptide of step a) can contain mostly natural amino acids, but also contain one or a small number of non-natural amino acids. Such a peptide is considered to consist essentially of natural amino acids.

In the screening method, the peptide of step b) will be the truncated or extended peptide of the invention as described above. In one embodiment, the structures of the non-natural amino acids of formulas I-V will be similar to those of the naturally occurring amino acids, lysine and arginine.

Thus, in the screening method contemplated herein, any extended or truncated peptide can be compared to any peptide having the same downstream sequence and having a known activity or pharmacological activity to determine whether or not the extended or truncated peptide has the same or similar activity or pharmacological activity at the same or different level. Depending on the specifics of how the measuring step is carried out, the present screening method can also be used to detect an activity or pharmacological activity exhibited by the extended or truncated peptide. Also, the screening method can be used to detect and measure qualitative and quantitative differences in the same or similar activity or pharmacological activity.

Thus, the methods of the present invention provide evaluation of the alteration of activity of the extended or truncated peptide. Typically, the hydrophobicity of the peptide is increased, which can result indirectly in increased binding activity when the desamino amino acid moiety is involved in binding (e.g., receptor-ligand binding, enzyme-cofactor binding, enzyme-substrate binding) and since binding strength is correlated with activity, a peptide higher potency (higher measured activity level) can result.

Furthermore, the desamino amino acids of the present invention also can enhance or increase the pharmacological activity of a peptide. For example, because the desamino amino acids are more hydrophobic (i.e., more lipophilic), a peptide containing a non-natural amino acid is more able to pass a body barrier (e.g., blood brain, blood ocular, skin, intestinal epithelium). Additionally, because the desamino amino acids impart increased selectivity and stability to a peptide, the pharmacological activity can also be screened when compared to other peptides.

### Treatment

The invention further relates to a method of treating or preventing in a subject a malcondition comprising administering to the subject an extended or truncated peptide having as its N-terminus an amino acid having the formula I-V. The basis peptide from which the extended or truncated peptide is formed will have or will be believed to have a biochemical, physiological, pharmacological or biological relationship with the malcondition to be treated or prevented. The malcondition may be a disease, biological or organic disfunction or an undesirable biological condition that is not ordinarily regarded as a disease or disfunction, such as but not limited to cosmetic malconditions such as skin blotches, acne, and the like. The subject may be a medical or veterinary patient including mammals such as humans, and non-humans mammamis such as dogs, cats, cows, sheep, pigs as well as avian.

In the method of the present invention, the malconditions that can be treated or prevented and the peptides that can be used are numerous. For example, a neurotensin peptide analogue that incorporates a non-natural amino acid of the invention can be synthesized. Such a synthetic NT peptide of the invention can be used to treat obesity, diabetes, sexual dysfunction, Parkingson's disease, atherosclerosis, insulin resistance, impaired glucose tolerance, hypercholesterolemia, or hypertrigylceridemia. A synthetic NT peptide of the invention can also be used to induce vasodilation, muscle relaxation, or to decrease appetite.

In addition, a synthetic NT peptide of the invention can be used to treat hyperthermia; hypothermia; gastrointestinal ulcers; substance abuse; depression; Alzheimer's disease; tardive dyskinesia; panic attack; gastrointestinal reflux disorder; irritable bowel syndrome; diarrhea; cholic; dyspepsia; pancreatitis; esophagitis; gastroparesis; neurological diseases such as schizophrenia, psychoses, anxiety, manic depression, delirium dementia, severe mental retardation, and dyskinesias such as Huntington's disease and Tourett's syndrome; fungal and viral infections, including HIV-1 and HIV-2 infections; pain (i.e., an analgesic); cancer (including gastrointestinal tumors); anorexia; bulimia; asthma; Parkinson's disease; acute heart failure; hypotension; hypertension; urinary retention; osteoporosis; angina pectoris; myocardial infarction; allergies; inflammation; or benign prostatic hypertrophy.

The methods of treatment of the present invention can also include combination therapy where other pharmaceutically active compounds useful for the treatment of obesity or other diseases are used in combination with a synthetic NT peptide of the invention.

It is known that obese patients have higher incidences of certain diseases such as atherosclerosis, hypercholesterolemia, hypertrigylceridemia, hypertension, sexual dysfunction (including erectile dysfunction), insulin resistance, impaired glucose tolerance, diabetes, [particularly non-insulin dependent diabetes mellitus (NIDDM or Type 2 diabetes)] and the diseases associated with diabetes such as nephropathy, neuropathy, retinopathy, cardiomyopathy, cataracts, and polycystic ovary syndrome. These diseases can be treated indirectly by treating obesity using a synthetic NT peptide of the invention or directly by treating the specific disease itself using a synthetic NT peptide of the invention. These diseases can be treated in the absence of obesity using a synthetic NT peptide of the invention.

In one embodiment of the invention, an obese patient or a patient at risk of becoming obese can be administered a combination of 1) a synthetic NT peptide of the invention; and 2) an additional compound useful to treat obesity, diabetes [including (NIDDM) and the conditions and/or diseases associated with diabetes, such as nephropathy, neuropathy, retinopathy, cardiomyopathy, cataracts, and polycystic ovary syndrome], atherosclerosis, hypercholesterolemia, hypertrigylceridemia, sexual dysfunction (including erectile dysfunction), insulin resistance, or impaired glucose tolerance, or combinations of compounds useful to treat these diseases.

Examples of classes of compounds that can be used to treat obesity include the active compound(s) in appetite suppressants such as Adipex®, Bontril®, Desoxyn Gradumet®, Fastin®, Ionamin®, and Meridia®, and lipase inhibitors such as Xenical®. Additional anti-obesity agents that can be used in combination with a synthetic NT peptide of the invention include a β3-adrenergic receptor agonist, a cholecystokinin-A agonist, a monoamine reuptake inhibitor, a sympathomimetic agent, a serotoninergic agent, a dopamine agonist, a melanocyte-stimulating hormone receptor agonist or mimetic, a melanocyte-stimulating hormone receptor analog, a cannabinoid receptor antagonist, a melanin concentrating hormone antagonist, leptin, a leptin analog, a leptin receptor agonist, a galanin antagonist, a bombesin agonist, a neuropeptide-Y antagonist (including NPY-1 and NPY-5), a thyromimetic agent, dehydroepiandrosterone or an analog thereof, a glucocorticoid receptor agonist or antagonist, an orexin receptor antagonist, a urocortin binding protein antagonist, a glucagon-like peptide-1 receptor agonist, and a ciliary neurotrophic factor.

In another aspect of the invention, a synthetic NT peptide of the invention can be administered in combination with a compound that is known to treat hypertension. Examples of classes of compounds that can be used to treat hypertension include calcium blockers, ACE inhibitors, diuretics, angiotensin II receptor blockers, .beta.-blockers, and .alpha.-adrenergic blockers. In addition, combinations of compounds in the above-recited classes have been used to treat hypertension. Some examples of specific compounds that can be used in combination with a synthetic NT peptide of the invention include quinapril; amlodipine, including the besylate salt; nifedipine; doxazosin, including the mesylate salt; and prazosin, including the hydrochloride salt.

In another aspect, a synthetic NT peptide of the invention can be used in combination with compounds useful for the treatment of diabetes, including impaired glucose tolerance, insulin resistance, insulin dependent diabetes mellitus (Type 1) and non-insulin dependent diabetes mellitus (NIDDM or Type 2). Also intended to be encompassed in the treatment of diabetes are the diabetic complications, such as neuropathy, nephropathy, retinopathy, cardiomyopathy or cataracts.

A synthetic NT peptide of the invention can also be used in combination with an aldose reductase inhibitor. Aldose reductase inhibitors constitute a class of compounds that have become widely known for their utility in treating conditions arising from complications of diabetes, such as diabetic neuropathy and nephropathy. Such compounds are well known to those skilled in the art and are readily identified by standard biological tests.

A synthetic NT peptide of the invention can also be used in combination with a sorbitol dehydrogenase inhibitor. Sorbitol dehydrogenase inhibitors lower fructose levels and have been used to treat or prevent diabetic complications such as neuropathy, retinopathy, nephropathy, cardiomyopathy, microangiopathy, and macroangiopathy.

A synthetic NT peptide of the invention can also be used in combination with a glucocorticoid receptor antagonist. The glucocorticoid receptor (GR) is present in glucocorticoid responsive cells where it resides in the cytosol in an inactive state until it is stimulated by an agonist. Upon stimulation the glucocorticoid receptor translocates to the cell nucleus where it specifically interacts with DNA and/or protein(s) and regulates transcription in a glucocorticoid responsive manner. GR antagonists can be used in the treatment of diseases associated with an excess or a deficiency of glucocorticoids in the body. As such, they may be used to treat the following: obesity, diabetes, cardiovascular disease, hypertension, Syndrome X, depression, anxiety, glaucoma, human immunodeficiency virus (HIV) or acquired immunodeficiency syndrome (AIDS), neurodegeneration (for example, Alzheimer's and Parkinson's), cognition enhancement, Cushing's Syndrome, Addison's Disease, osteoporosis, frailty, inflammatory diseases (such as osteoarthritis, rheumatoid arthritis, asthma and rhinitis), adrenal dysfunction, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism and prevention of muscle frailty.

In addition, a synthetic NT peptide of the invention can be administered in combination with other pharmaceutical agents such as cholesterol biosynthesis inhibitors and cholesterol absorption inhibitors, especially HMG-CoA reductase inhibitors and HMG-CoA synthase inhibitors, HMG-CoA reductase and synthase gene expression inhibitors, CETP inhibitors, bile acid sequesterants, fibrates, ACAT inhibitors, squalene synthetase inhibitors, anti-oxidants and niacin. A a synthetic NT peptide of the invention may also be administered in combination with naturally occurring compounds that act to lower plasma cholesterol levels. These naturally occurring compounds are commonly called nutraceuticals and include, for example, garlic extract, Benecol®, and niacin.

It is also contemplated that a synthetic NT peptide of the invention be administered with a lipase inhibitor and/or a glucosidase inhibitor, which are typically used in the treatment of conditions resulting from the presence of excess triglycerides, free fatty acids, cholesterol, cholesterol esters or glucose including, inter alia, obesity, hyperlipidemia, hyperlipoproteinemia, Syndrome X, and the like.

Any lipase inhibitor or glucosidase inhibitor may be used in a combination with a synthetic NT peptide of the invention. Preferred lipase inhibitors comprise gastric or pancreatic lipase inhibitors such as orlistat. Preferred glucosidase inhibitors comprise amylase inhibitors. A lipase inhibitor is a compound that inhibits the metabolic cleavage of dietary triglycerides into free fatty acids and monoglycerides. Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a monoglyceride and a fatty acid. The resultant free fatty acids and monoglycerides are incorporated into bile acid-phospholipid micelles, which are subsequently absorbed at the level of the brush border of the small intestine. The micelles eventually enter the peripheral circulation as chylomicrons. Accordingly, compounds, including lipase inhibitors that selectively limit or inhibit the absorption of ingested fat precursors are useful in the treatment of conditions including obesity, hyperlipidemia, hyperlipoproteinemia, Syndrome X, and the like. A variety of lipase inhibitors are known to one of ordinary skill in the art.

Any glucosidase inhibitor may be used in combination with a synthetic NT peptide of the invention, however, a generally preferred glucosidase inhibitor is an amylase inhibitor. An amylase inhibitor is a glucosidase inhibitor that inhibits the enzymatic degradation of starch or glycogen into maltose. The inhibition of such enzymatic degradation is beneficial in reducing amounts of bioavailable sugars, including glucose and maltose, and the concomitant deleterious conditions resulting therefrom. Glucosidase and amylase inhibitors are known to one of ordinary skill in the art.

In addition, the present invention includes the use of a synthetic NT peptide of the invention in combination with apo B secretion/MTP inhibitors. A variety of apo B secretion/MTP inhibitors are known to one of ordinary skill in the art. A synthetic NT peptide of the invention also can be used in combination with one or more additional compounds that are neurotensin receptor ligands.

Non-natural amino acids of the invention can also be incorporated into other peptides to treat malconditions as set out below.

Peptides for triggering B and T cell activity can be used to treat autoimmune disease, including uveitis, collagen-induced, adjuvant and rheumatoid arthritis, thyroiditis, myasthenia gravis, multiple sclerosis and diabetes. Examples of these peptides are interleukins (referenced in Aulitzky, W E; Schuler, M; Peschel, C.; Huber, C.; Interleukins. Clinical pharmacology and therapeutic use. Drugs. 48(5):667-77, 1994 November) and cytokines (referenced in Peters, M.; Actions of cytokines on the immune response and viral interactions: an overview. Hepatology. 23(4):909-16, 1996 April).

Enkephlin and analogs, agonists and antagonists can be used to treat AIDS, ARC, and cancer, pain modulation, Huntington's, Parkinson's diseases.

LHRH and analogs, agonists and antagonists can be used to treat prostatic tumors and reproductive physiopathology, including breast cancer, and infertility.

Peptides and peptidomimetics that target crucial enzymes, oncogenes or oncogene products, tumor-suppressor genes and their products, growth factors and their corresponding receptors can be used to treat cancer. Examples of these peptides are described in Unger, C. Current concepts of treatment in medical oncology: new anticancer drugs. Journal of Cancer Research & Clinical Oncology. 122(4):189-98, 1996.

Neuropeptide Y and other pancreatic polypeptides, and analogs, agonists and antagonists can be used to treat stress, anxiety, depression and associated vasoconstrictive activities.

Gluco-incretins, including gastric inhibitory polypeptide, glucose-dependent insulinotropic polypeptide, PACAP/Glucagon and glucagon-like polypeptide-1 and 2 and analogs, agonists and antagonists can be used to treat Type II diabetic hyperglycaemia.

Atrial natriuretic factor and analogs, agonists and antagonists can be used to treat congestive heart failure.

Integrin and analogs, agonists and antagonists can be used to treat osteoporosis, scar formation, bone synthesis, inhibition of vascular occlusion, and inhibition of tumor invasion and metastasis.

Glucagon, glucagon-like peptide 1, PACAP/Glucagon, and analogs, agonists and antagonists can be used to treat diabetes cardiovascular emergencies.

Antithrombotic peptides and analogs, agonists and antagonists can be used to treat cardiovascular and cerebrovascular diseases. Examples of these peptides RGD, D-Phe-Pro-Arg and others named are described in Ojima I.; Chakravarty S.; Dong Q. Antithrombotic agents: from RGD to peptide mimetics. Bioorganic & Medicinal Chemistry. 3(4):337-60, 1995.

Cytokines/interleukins and analogs, agonists and antagonists can be used to treat inflammatory disease, immune response dysfunction, hematopoiesis, mycosis fungoides, aplastic anemia, thrombocytopenia, and malignant melanoma. Examples of these peptides are Interleukins, referenced in Aulitzky et al. and Peters at al.

Endothelin and analogs, agonists and antagonists can be used to treat arterial hypertension, myocardial infarction, congestive heart failure, atherosclerosis, shock conditions, renal failure, asthma and vasospasm

Natriuretic hormones and analogs, agonists and antagonists can be used to treat cardiovasicular disease and acute renal failure. Examples of these peptides are named and described in Espiner, E. A; Richards, A. M.; Yandle, T. G.; Nicholls, M. G.; Natriuretic hormones. Endocrinology & Metabolism Clinics of North America. 24(3):481-509, 1995.

Peptides that activate or inhibit tyrosine kinase, or bind to TK-activating or inhibiting peptides and analogs, agonists and antagonists can be used to treat chronic myelogenous and acute lymphocytic leukemias, breast and ovarian cancers and other tyrosine kinase associated diseases. Examples of these peptides are described in Smithgall, T E.; SH2 and SH3 domains: potential targets for anti-cancer drug design. Journal of Pharmacological & Toxicological Methods. 34(3):125-32, 1995.

Renin inhibitors analogs, agonists and antagonists can be used to treat cardiovascular disease, including hypertension and congestive heart failure. Examples of these peptides are described in Rosenberg, S. H.; Renin inhibition. Cardiovascular Drugs & Therapy. 9(5):645-55, 1995.

Angiotensin-converting enzyme inhibitors, analogs, agonists and antagonists can be used to treat cardiovascular disease, including hypertension and congestive heart failure.

Peptides that activate or inhibit tyrosine phosphorylases can be used to treat cardiovascular diseases. Examples of these peptides are described in Srivastava, A. K.; Protein tyrosine phosphorylation in cardiovascular system. Molecular & Cellular Biochemistry. 149-150:87-94, 1995.

Peptide based antivirals can be used to treat viral diseases. Examples of these peptides are described in Toes, R. E.; Feltkamp, M. C.; Ressing, M. E.; Vierboom, M. P.; Blom, R. J.; Brandt, R. M; Hartman, M.; Offringa, R.; Melief, C. J.; Kast, W. M.; Cellular immunity against DNA tumour viruses: possibilities for peptide-based vaccines and immune escape. Biochemical Society Transactions. 23(3):692-6, 1995.

Corticotropin releasing factor and peptide analogs, agonists and antagonists can be used to treat disease associated with high CRF, i.e Alzheimer's disease, anorexia nervosa, depressive disorders, arthritis, and multiple sclerosis.

Peptide agonists and antagonists of platelet-derived wound-healing formula (PDWHF) can be used as a therapy for donor tissue limitations and wound-healing constraints in surgery. Examples of these peptides are described in Rudkin, G. H.; Miller, T. A.; Growth factors in surgery. Plastic & Reconstructive Surgery. 97(2):469-76, 1996.

Fibronectin, fibrinopeptide inhibitors and analogs, agonists and antagonists can be used to treat metastasis (i.e. enzyme inhibition, tumor cell migration, invasion, and metastasis).

Chemokine (types of cytokine, including interleukin-8, RANTES, and monocyte chemotactic peptide) analogs, agonists and antagonists can be used to treat arthritis, hypersensitivity, angiogenesis, renal disease, glomerulonephritis, inflammation, and hematopoiesis.

Neutral endopeptidase inhibitors and analogs, agonists and antagonists can be used to treat hypertension and inflammation. Examples of these peptides are described in Gregoire, J. R; Sheps, S. G; Newer antihypertensive drugs. Current Opinion in Cardiology. 10(5):445-9, 1995.

Substance P and analogs, agonists and antagonists can be used to treat immune system dysfunction, pain transmission/perception and in autonomic reflexes and behaviors.

Alpha-melanocyte-stimulating hormone and analogs, agonists and antagonists can be used to treat AIDS, rheumatoid arthritis, and myocardial infarction.

Bradykinin (BK) and analogs, agonists and antagonists can be used to treat inflammatory diseases (edema, etc), asthma, allergic reactions (rhinitis, etc), anesthetic uses, and septic shock.

Secretin can be used to treat cardiovascular emergencies.

GnRH and analogs, agonists and antagonists can be used to treat hormone-dependent breast and prostate tumors.

Somatostatin and analogs, agonists and antagonists can be used to treat gut neuroendocrine tumors.

Gastrin, Gastrin Releasing Peptide and analogs, agonists and antagonists can be used as an adjuvant to chemotherapy or surgery in small cell lung cancer and other malignancies, or to treat allergic respiratory diseases, asthma and allergic rhinitis.

Laminin, the Laminin derivative antimetastatic drug YIGSR peptide, Laminin-derived synthetic peptides analogs, agonists and antagonists can be used to treat tumor cell growth, angiogenesis, regeneration studies, vascularization of the eye with diabetes, and ischemia. The peptides of this category can inhibit the tumor growth and metastasis of leukemic cells and may be useful as a potential therapeutic reagent for leukaemic infiltrations. Peptides containing this sequence also inhibit experimental metastasis. Exemplary references include McGowan K A. Marinkovich M P. Laminins and human disease. Microscopy Research & Technique. 51(3):262-79, 2000 Nov. 1; Yoshida N. Ishii E. Nornizu M. Yamada Y. Mohri S. Kinukawa N. Matsuzaki A. Oshima K. Hara T. Miyazaki S. The laminin-derived peptide YIGSR (Tyr-Ile-Gly-Ser-Arg) inhibits human pre-B leukaemic cell growth and dissemination to organs in SCID mice. British Journal of Cancer. 80(12):1898-904, 1999. Examples of these peptides are also described in Kleinman, H. K.; Weeks, B. S.; Schnaper, H. W.; Kibbey, M. C.; Yamamura, K.; Grant, D. S; The laminins: a family of basement membrane glycoproteins important in cell differentiation and tumor metastases. Vitamins & Hormones. 47:161-86, 1993.

Defensins, corticostatins, dermaseptins, mangainins, and other antibiotic (antibacterial and antimicrobial) peptides and analogs, agonists and antagonists can be used to treat infections, tissue inflammation and endocrine regulation.

Vasopressin and analogs, agonists and antagonists can be used to treat neurological disorders, stress and Diabetes insipidus.

Oxytocin and analogs, agonists and antagonists can be used to treat neurological disorders and to induce labor.

ACTH-related peptides and analogs, agonists and antagonists can be used as neurotrophic, neuroprotective, and peripheral demyelinating neuropathy agents.

Amyloid-beta peptide and analogs, agonists and antagonists can be used to treat Alzheimer's disease.

Epidermal growth factor, receptor, and analogs, agonists and antagonists can be used to treat necrotizing enterocolitis, Zollinger-Ellison syndrome, gastrointestinal ulceration, colitis, and congenital microvillus atrophycarcinomas.

Leukocyte adhesion molecules and their ligands, and analogs, agonists and antagonists can be used to treat atherosclerosis, inflammation. Examples of these peptides are described in Barker, J. N.; Adhesion molecules in cutaneous inflammation. Ciba Foundation Symposium. 189:91-101.

Major histocompatibility complex (MHC) binding peptides and analogs, agonists and antagonists can be used to treat autoimmune, immunodysfunctional, immuno modulatory diseases and as well as used for their corresponding therapies. Examples of these peptides are described in Appella, E.; Padlan, E. A.; Hunt, D. F; Analysis of the structure of naturally processed peptides bound by class I and class II major histocompatibility complex molecules. EXS. 73:105-19, 1995.

Corticotropin releasing factor can be used to treat neurological disorders.

Neurotrophins (including brain-derived neurotrophic factor (BDNF), nerve growth factor, and neurotrophin 3) and analogs, agonists and antagonists can be used to treat neurological disorders.

Cytotoxic T-cell activating peptides can be used to treat infectious diseases and cancer. Examples of these peptides are described in: Chesnut R. W.; Sette, A.; Celis, E.; Wentworth, P.; Kubo, R.T.; Alexander, J.; Ishioka, G.; Vitiello, A.; Grey, H. M; Design and testing of peptide-based cytotoxic T-cell-mediated immunotherapeutics to treat infectious diseases and cancer. Pharmaceutical Biotechnology. 6:847-74, 1995.

Peptide immunogens for prevention of HIV-1 and HTLV-I retroviral infections can be used to treat AIDS. Examples of these peptides are described in Hart, M. K.; Palker, T. J.; Haynes, B F; Design of experimental synthetic peptide immunogens for prevention of HIV-1 and HTLV-I retroviral infections. Pharmaceutical Biotechnology. 6:821-45, 1995.

Galanin and analogs, agonists and antagonists can be used to treat Alzheimer's disease, depression, eating disorders, chronic pain, prevention of ischemic damage, and growth hormone modulation.

Tachykinins (neurokinin A and neurokinin B) and analogs, agonists and antagonists can be used to treat pain transmission/perception and in autonomic reflexes and behaviors.

RGD containing peptides can be used to treat various diseases involved with cell adhesion, antithrombotics, and acute renal failure.

Osteogenic growth peptide and analogs, agonists and antagonists can be used as treatment of systemic bone loss. Examples of these peptides are described in Bab IA. Regulatory role of osteogenic growth peptide in proliferation, osteogenesis, and hemopoiesis. Clinical Orthopaedics & Related Research. (313):64-8, 1995.

Parathyroid hormone, parathyroid hormone related-peptide and analogs, agonists and antagonists can be used to treat diseases affecting calcium homeostasis (hypercalcemia), bone metabolism, vascular disease, and atherosclerosis.

Kallidin and analogs, agonists and antagonists can be used to treat tissue injury or inflammation and pain signaling pathological conditions of the CNS.

T cell receptor peptide vaccines and analogs, agonists and antagonists can be used in immunotherapy. Examples of these peptides are described in Brostoff, S W; T cell receptor peptide vaccines as immunotherapy. Agents & Actions—Supplements. 47:53-8, 1995.

Platelet-derived growth factor (PDGF) and analogs, agonists and antagonists can be used to treat non-neoplastic hyperproliferative disorders, therapy for donor tissue limitations and wound-healing constraints in surgery.

Amylin, calcitonin gene related peptides (CGRP) and analogs, agonists and antagonists can be used to treat insulin-dependent diabetes.

Vasoactive intestinal polypeptide and analogs, agonists and antagonists can be used to treat allergic respiratory diseases, asthma and allergic rhinitis, and nervous control of reproductive functions.

Growth hormone-releasing hormone and analogs, agonists and antagonists can be used to treat growth hormone deficiency and immunomodulation.

HIV protease inhibiting peptides can be used to treat AIDS. Examples of these peptides are described in Bugelski, P. J.; Kirsh, R.; Hart, T. K; HIV protease inhibitors: effects on viral maturation and physiologic function in macrophages. Journal of Leukocyte Biology. 56(3):374-80, 1994.

Thymopoietin active fragment peptides and analogs, agonists and antagonists can be used to treat rheumatoid arthritis and virus infections.

Cecropins and analogs, agonists and antagonists can be used as antibacterials.

Thyroid releasing hormone and analogs, agonists and antagonists can be used to treat spinal cord injury and shock.

Erythropoietin and analogs, agonists and antagonists can be used to treat anemia.

Fibroblast growth factor (FGF), receptor and analogs, agonists and antagonists can be as stimulation of bone formation, as well as used as a treatment for Kaposi's sarcoma, neuron regeneration, prostate growth, tumor growth inhibition, and angiogenesis.

Stem cell factor and analogs, agonists and antagonists can be used to treat anemias.

GP120, GP160, CD4 fragment peptides and analogs, agonists and antagonists can be used to treat AIDS.

Insulin-like growth factor, receptor, and analogs, agonists and antagonists can be used to treat breast and other cancers, noninsulin-dependent diabetest mellitus, cell proliferation, apoptosis, hematopoiesis, AIDS, growth disorders, osteoporosis, and insulin resistance.

Colony stimulating factors (granulocyte-macrophage colony-stimulating factor, granulocyte colony-stimulating factor, and macrophage colony-stimulating factor and analogs, agonists and antagonists can be used to treat anemias.

Kentsin and analogs, agonists and antagonists can be used for immunomodulation.

Lymphocyte activating peptide and analogs, agonists and antagonists can be used for immunomodulation. Examples of these peptides are described in Loleit, M.; Deres, K.; Wiesmuller, K. H.; Jung, G.; Eckert, M.; Bessler, W. G; Biological activity of the Escherichia coli lipoprotein: detection of novel lymphocyte activating peptide segments of the molecule and their conformational characterization. Biological Chemistry Hoppe-Seyler. 375(6):407-12, 1994 June.

Tuftsin and analogs, agonists and antagonists can be used for immunomodulation.

Prolactin and analogs, agonists and antagonists can be used to treat rheumatic diseases, systemic lupus erythematosus, and hyperprolactemia.

Angiotensin II and receptor(s) and analogs, agonists and antagonists can be used to treat hypertension, hemodynamic regulation, neurological disorders, diabetic nephropathies, aortoarterities induced RVH, hyperaldosteronism, heavy metal induced cardiovascular effects, diabetes mellitus and thyroid dysfunction.

Dynorphin and analogs, agonists and antagonists can be used to treat neurological disorders, pain management, algesia, spinal cord injury and epilepsy.

Calcitonin, and analogs, agonists and antagonists can be used to treat neurological disorders, immune system dysfunction, calcium homeostasis, and osteoporosis.

Pituitary adenylate cyclase activating polypeptide can play a role in growth, signal transduction vasoactivity roles, exact role in diseases not determined yet.

Cholecystokinin and analogs, agonists and antagonists can be used to treat feeding disorders, panic disorders, and anti-opioid properties.

Pepstatin and analogs, agonists and antagonists can be used as pepsin and HIV protease inhibitors (AIDS).

Bestatin and analogs, agonists and antagonists can be used to treat muscular dystrophy, anticancer, antileukemia, immune response modulator, and acute non-lymphocytic leukemia.

Leupeptin and analogs, agonists and antagonists can be used as a protease inhibitor, exact role in diseases not determined yet.

Luteinizing hormone and releasing hormone and analogs, agonists and antagonists can be used as a infertility male contraceptive.

Neurotensin and analogs, agonists and antagonists can be used, e.g., as antipsychotic, analgesic, anti-cancer, and/or neuroprotective agents, e.g., for treating stroke victims, e.g., by inducing hypothermia so as to provide neuroprotection.

Motilin and analogs, agonists and antagonists can be used for the control of gastric emptying.

Insulin and analogs, agonists and antagonists can be used to treat diabetes.

Transforming growth factor (TGF) and analogs, agonists and antagonists can be used for cell proliferation and differentiation, cancer treatment, immunoregulation, therapy for donor tissue limitations, and wound-healing constraints in surgery.

Bone morphogenetic proteins (BMPs) and analogs, agonists and antagonists can be used as therapy for donor tissue limitations, osteogenesis, and wound-healing constraints in surgery.

Bombesin and Enterostatin as well as their analogs, agonists and antagonists can be used to prevent the proliferation of tumor cells, modulation of feeding, and neuroendocrine functions. These peptides fall within a supercategory of the neuromedins described above. These peptides are described in such exemplary references as Yamada K. Wada E. Wada K. Bombesin-like peptides: studies on food intake and social behaviour with receptor knock-out mice. Annals of Medicine. 32(8):519-29, 2000 November; Ohki-Hamazaki H. Neuromedin B. Progress in Neurobiology. 62(3):297-312, 2000 October; Still CD. Future trends in weight management. Journal of the American Osteopathic Association. 99(10 Su Pt 2):S18-9, 1999; Martinez V. Tache Y. Bombesin and the brain-gut axis. Peptides. 21(11):1617-25, 2000; Afferent signals regulating food intake. Proceedings of the Nutrition Society. 59(3):373-84, 2000; Takenaka Y. Nakamura F. Jinsmaa Y. Lipkowski A W. Yoshikawa M. Enterostatin (VPDPR) has anti-analgesic and anti-amnesic activities. Bioscience Biotechnology & Biochemistry. 65(1):236-8, 2001 J.

Glucagon, glucagon-like peptide 1 and analogs, agonists and antagonists can be used to treat diabetes cardiovascular emergencies.

Pancreastatin, chromogranins A, B and C and analogs, agonists and antagonists—conditions associated with inhibition of insulin secretion, exocrine pancreatic secretion and gastric acid secretion, and stimulation of egradati secretion.

Endorphins and analogs, agonists and antagonists can be used to treat neurological disorders, alleviating pain, treatment of opioid abuse, obesity, and diabetes. Examples of these peptides are named and described in Dalayeun, J. F.; Nores, J. M.; Bergal, S.; Physiology of beta-endorphins. A close-up view and a review of the literature. Biomedicine & Pharmacotherapy. 47(8):311-20, 1993.

Miscellaneous opioid peptides, including (but not limited to) adrenal peptide E, alpha casein fragment, beta casomorphin, dermorphin, kyotorphin, metophamide neuropeptide FF (NPFF), melanocyte inhibiting factor, and analogues, agonists and antagonists can be used to treat neurological disorders, alleviating pain, as well as for the treatment of opioid abuse.

Vasotocin and analogues, agonists and antagonists can be used for clinical uses to be determined.

Protein kinase C and inhibitors and analogues, agonists and antagonists can be used to treat cancer, apoptosis, smooth muscle function, and Alzheimer's disease. Examples of these peptides are named and described in Philip, P. A.; Harris, A. L; Potential for protein kinase C inhibitors in cancer therapy. Cancer Treatment & Research. 78:3-27, 1995.

Amyloid, amyloid fibrin, fragments and analogues, agonists and antagonists can be used to treat neurodegenerative diseases and diabetes.

Calpain and other calmodulin-inhibitory proteins and analogues, agonists and antagonists can be used to treat neurodegenerative disorders, cerebral ischaemia, cataracts, myocardial ischaemia, muscular dystrophy and platelet aggregation.

Charybdotoxin, Apamin and analogues, agonists and antagonists can be used for treatment of neurodegenerative diseases and pain and cerebral ischemia.

Phospholipase A2 and receptor inhibiting/activating peptides and analogues, agonists and antagonists can be used to treat acute pancreatitis, pancreatic cancer, abdominal trauma, and inflammation, e.g., sepsis, infections, acute pancreatitis, various forms of arthritis, cancer, complications of pregnancy, and postoperative states.

Potassium channel activating and inhibiting proteins and analogues, agonists and antagonists can be used to treat various diseases. Examples of these peptides are described in Edwards, G.; Weston, A. H; Pharmacology of the potassium channel openers. Cardiovascular Drugs & Therapy. 9 Suppl 2:185-93, 1995 March.

IgG activators, inhibitors and analogues, agonists and antagonists can be used to treat autoimmune diseases and immune dysfunctions. Examples of these peptides are described in Mouthon, L.; Kaveri, S N.; Spalter, S. H.; Lacroix-Desmazes, S.; Lefranc, C.; Desai, R.; Kazatchkine, M. D; Mechanisms of action of intravenous immune globulin in immune-mediated diseases. Clinical & Experimental Immunology. 104 Suppl 1:3-9, 1996.

Endotoxin and inhibitors and analogues, agonists and antagonists can be used for decreasing cardiac output, systemic hypotension, decreased blood flow and O₂ delivery to tissues, intense pulmonary vasoconstriction and hypertension, bronchoconstriction, increased permeability, pulmonary oedema, ventilation-to-perfusion inequalities, hypoxaemia, and haemoconcentration. Examples of these peptides are named and described in Burrell, R; Human responses to bacterial endotoxin. Circulatory Shock. 43(3):137-53, 1994 July.

Orphan receptor ligands (including but not limited to ADNF, Adrenomedullin, Apelin, Ghrelin, Mastoparan (MCD peptides), Melanin concentrating hormone, Nociceptin/Nocistatin, Orexin, Receptor activity modulating protein, Urotensin). By definition, orphan receptors do not have a function associated with them, but are considered to be key players in future drug development. These orphan receptor ligands are described in such references as In DS. Orphan G protein-coupled receptor s and beyond. Japanese Journal of Pharmacology. 90(2):101-6, 2002; Maguire J J. Discovering orphan receptor function using human in vitro pharmacology. Current Opinion in Pharmacology. 3(2):135-9, 2003; Szekeres P G. Functional assays for identifying ligands at orphan G protein-coupled receptor s. Receptor s & Channels. 8(5-6):297-308, 2002; Shiau A K. Coward P. Schwarz M. Lehmann J M. Orphan nuclear receptor s: from new ligand discovery technologies to novel signaling pathways. Current Opinion in Drug Discovery & Development. 4(5):575-90, 2001; Civelli O. Nothacker H P. Saito Y. Wang Z. Lin S H. Reinscheid R K. Novel neurotransmitters as natural ligands of orphan G-protein-coupled receptor s. Trends in Neurosciences. 24(4):230-7, 2001; Darland T. Heinricher M M. Grandy D K. Orphan in FQ/nociceptin: a role in pain and analgesia, but so much more. Trends in Neurosciences. 21(5):215-21, 1998, the disclosures of which are incorporated herein by reference.

Another category includes Glycoprotein IIb/IIIa inhibitors. The central role of platelet-rich thrombus in the pathogenesis of acute coronary syndromes (ACSs) is well-known. Glycoprotein IIb/IIIa (Gp IIb/IIIa) receptor antagonists are potent inhibitors of platelet function that may be expected to affect favorably the natural history of ACSs. Exemplary references for this category include Bhatt D L. Topol E J. Current role of platelet glycoprotein IIb/IIIa inhibitors in acute coronary syndromes. JAMA. 284(12):1549-58, 2000; Kereiakes D J. Oral blockade of the platelet glycoprotein IIb/IIIa receptor: fact or fancy?. American Heart Journal. 138(1 Pt 2):S39-46, 1999; Bassand J P. Low-molecular-weight heparin and other antithrombotic agents in the setting of a fast-track revascularization in unstable coronary artery disease. Haemostasis. 30 Suppl 2:114-21; discussion 106-7, 2000.

### The Use of Peptides Containing Desamino Amino Acid(s) to Pass a Body Barrier

The invention relates to a method of increasing the ability of a peptide to cross a body barrier of a subject by use of the extended or truncated peptide having as its N-terminus a residue of the compound formula I-V.

The invention further relates to a method of treating or preventing in a subject a disease or condition treated or prevented by the administration of an extended or truncated peptide, whereby the extended or truncated peptide crosses the body barrier in higher amounts than the peptide having no non-natural amino acid.

The invention also relates to a method of treating or preventing in a subject a disease or condition of the brain treated or prevented by the administration of an extended or truncated peptide.

The use of peptides as therapeutic agents is limited by their inability to cross body barriers. The phrase “body barrier” is defined herein as a cellular membrane or other structure that functions to prevent free (e.g., diffusional) passage of certain molecules. The use of an extended or truncated peptide of the invention facilitates the passage of the resultant peptide through a variety of body barriers. Examples of body barriers include, but are not limited to, the blood brain barrier, a cell membrane, intestinal epithelium, skin cell, or the blood—ocular. In a preferred embodiment, the body barrier is the blood brain barrier.

### Selectivity and Stability of Peptides Containing Non-Natural Amino Acid(s)

Certain embodiments of the invention relate to a method of increasing the selectivity of a chosen peptide through use of an extended or truncated peptide based upon the sequence of the chosen peptide as described above.

Enhancing the selectivity of a drug to a biological target is of great importance. In one embodiment, a peptide containing arginine and/or lysine can be converted according to the invention into an extended or truncated peptide in order to increase the selectivity of the peptide. In another embodiment, any of the non-natural amino acids disclosed herein can be used to increase the selectivity of a peptide.

### Pharmaceutical Composition

The peptides of the invention can be used in any therapeutic procedure available to one of skill in the art to treat any disease or physiological problem with which the corresponding known peptide is associated.

The peptides of the invention can be formulated as pharmaceutical compositions and administered to a mammalian host, such as a human patient, in a variety of dosage forms adapted to the chosen route of administration, i.e., orally or parenterally, by intravenous, intramuscular, topical or subcutaneous routes.

Thus, the peptides may be systemically administered, for example, intravenously or intraperitoneally by infusion or injection. Solutions of the peptide or peptide conjugate can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient(s) that are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the peptide or peptide conjugate in the required amount in the appropriate solvent with various of the other ingredients enumerated above, as required, followed by filter sterilization. In the case of sterile powders for the preparation of sterile injectable solutions, the preferred methods for preparation of such powders are vacuum drying and freeze drying techniques, which yield a powder of the active ingredient plus any additional desired ingredient present in the previously sterile-filtered solutions.

In some instances, the peptides of the invention can also be administered orally, in combination with a pharmaceutically acceptable vehicle such as an inert diluent or an assimilable edible carrier. The peptides may be enclosed in hard or soft shell gelatin capsules, may be compressed into tablets, or may be incorporated directly with the food of the patient's diet. For oral therapeutic administration, the peptide or peptide conjugate may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% to about 90% of the weight of a given unit dosage form. The amount of peptide in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the peptides of the invention may be incorporated into sustained-release preparations and devices.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the peptides of the invention can be determined by correlating their in vitro activity, and in vivo activity in animal models described herein.

The therapeutically effective amount of peptide of the invention necessarily varies with the subject and the disease or physiological problem to be treated and correlates with the effective amounts of the corresponding known peptide. For example, a therapeutic amount between 30 to 112,000 μg per kg of body weight can be effective for intravenous administration. As one skilled in the art would recognize, the amount can be varied depending on the method of administration. The amount of the peptide of the invention, required for use in treatment will also vary with the route of administration, but also the nature of the condition being treated and the age and condition of the patient and will be ultimately at the discretion of the attendant physician or clinician.

The compound can conveniently be administered in unit dosage form; for example, containing 1 to 1000 mg, conveniently 10 to 750 mg, most conveniently, 20 to 500 mg of peptide per unit dosage form.

Ideally, the peptide should be administered to achieve peak plasma concentrations of from about 0.1 to about 75 μM, preferably, about 1 to 50 μM, most preferably, about 2 to about 30 μM. This may be achieved, for example, by the intravenous injection of a 0.05 to 5% solution of the peptide, optionally in saline, or orally administered as a bolus containing about 1-100 mg of the peptide. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose, as divided doses, or as a continuous infusion. The desired dose can also be administered at appropriate intervals, for example, as two, three, four or more sub-doses per day.

### Cosmetic Formulation

An important role for makeup cosmetic is “beautification” or making the appearance more beautiful. Often which role involves correction of skin roughness, blemishes and color as well as vitality.

A cosmetic composition of the present invention contains the typical and common base carriers as well as a desamino amino acid compound of the invention. Usually the compound of the invention will be in the form of an ester, amide or salt for this purpose. Generally, the cosmetic base will depend upon the kind of make-up being formulated: face creme, face powder, pancake make-up, skin creme, lip stick, rouge and the like. These bases will contain appropriate, nontoxic colorants, emuliants, oils, waxes, solvents, emulsifiers, fatty acids, alcohols or esters, gums, inorganic inert builders and the like.

For example, the gums, may include various known polysaccharide compounds, for example, cellulose, hemicellulose, gum arabic, tragacanth gum, tamarind gum, pectin, starch, mannan, guar gum, locust bean gum, quince seed gum, alginic acid, carrageenan, agar, xanthane gum, dextran, pullulan, chitin, chitosan, hyaluronic acid, chondroitin sulfuric acid, etc., derivatives of polysaccharide compounds, for example, carboxymethylated derivatives, sulfate derivatives, phosphated derivatives, methylated derivatives, ethylated derivatives, addition derivatives of alkylene oxide such as ethylene oxide or propylene oxide, acylated derivatives, cationated derivatives, low molecular weight derivatives, and other polysaccharide derivatives may be mentioned.

Another component which may be included in the external composition of the present invention is a powder component. Powder components based on inorganic components such as talc, kaolin, mica, sericite, dolomite, phlogopite, synthetic mica, lepidolite, biotite, lithia mica, vermiculite, magnesium carbonate, calcium carbonate, aluminum silicate, barium silicate, calcium silicate, magnesium silicate, strontium silicate, metal salts of tungstenic acid, magnesium, silica, zeolite, barium sulfate, sintered calcium sulfate (sintered gypsum), calcium phosphate, fluorapatite, hydroxyapatite, ceramic powder, metal soap (zinc myristate, calcium palmitate, ammonium stearate), boronitride, etc.; and organic powder components such as polyamide resin powder (nylon powder), polyethylene powder, polymethyl methacrylate powder, polystyrene powder, copolymer resin powder of styrene and acrylic acid, benzoguanamine resin powder, silicone resin powder, silicone rubber powder, silicone resin covered rubber powder, polyethylene tetrafluoride powder, cellulose powder, etc. may be mentioned.

Further, powder components obtained by treating the surfaces of these powder components by a silicone compound, fluorine-modified silicone compound, fluorine compound, higher aliphatic acid, higher alcohol, aliphatic acid ester, metal soap, alkyl phosphate, etc. may be formulated into the external composition of the present invention depending upon the need.

The known dyes or pigments, may be used. For example, inorganic white pigments such as titanium dioxide, zinc oxide, inorganic red pigments such as iron oxide (bengala), iron titanate, inorganic brown pigments such as .gamma.-iron oxide; inorganic yellow pigments such as yellow iron oxide, yellow earth; inorganic black pigments such as black iron oxide, carbon black, lower titanium oxide, and; inorganic violet pigments such as mango violet, cobalt violet; inorganic green pigments such as chromium oxide, chromium hydroxide, cobalt titanate; blue pigments such as prussian blue, ultramarine; pearl pigments such as titanium oxide coated mica, titanium oxide coated bismuth oxichloride, titanium oxide coated talc, colored titanium oxide coated mica, bismuth oxichloride, fish scales; metal powder pigments such as aluminum powder, copper powder; organic pigments of zirconium, barium or aluminum lakes etc. such as Lithol rubine B (Red No. 201), Lithol rubine BCA (Red No. 202), Lake red CBA (Red No. 204), Lithol red (Red No. 205), Deep maroon (Red No. 220), Helidone pink CN (Red No. 226), Permatone Red (Red No. 228), Permanent red F5R (Red No. 405), Permanent orange (Orange No. 203), Benzidine Orange (Orange No. 204), Benzidine yellow G (Yellow No. 205), Hanza Yellow (Yellow No. 401), Blue No. 404, and other organic pigments; Erythrosine (Red No. 3), Phloxine B (Red No. 104), Acid red (Red No. 106), Fast acid magenta (Red No. 227), Eosine YS (Red No. 230), Violamine R (Red No. 401), Oil red XO (Red No. 505), Orange II (Orange No. 205), Tartrazine (Yellow No. 4), Sunset yellow FCF (Yellow No. 5), Uranine (Yellow No. 202), Quinoline yellow (Yellow No. 203), Fast green FCF (Green No. 3), Brilliant blue FCF (Blue No. 1) may be mentioned.

The cosmetic composition of the present invention may be formulated with a liquid. As the liquid, it is possible to select a volatile component ordinarily used in external compositions such as cosmetics. Specifically, it is possible to mention, for example, volatile silicone oil, water, or a lower alcohol (or mixtures of the same). These volatile components may be suitably selected depending upon the specific form of the external composition of the present invention (for example, the later mentioned “roughness correcting composition” or “makeup composition” etc.) or type of carrier (for example, oil base or emulsion base etc.). By formulating these volatile components, it is possible to adjust the viscosity of the product at the time of use of the external composition of the present invention and adjust the thickness of coating of the external composition on the skin.

As the volatile silicone oil, it is possible to use a volatile silicone oil which is used in the field of cosmetics and other external compositions. It is not particularly limited. Specifically, for example, a low boiling point linear silicone oil such as hexamethyl disiloxane, octamethyl trisiloxane, decamethyl cyclopentasiloxane, dodecamethyl cyclohexasiloxane, and tetradecamethyl cycloheptasiloxane; a low boiling point cyclic silicone oil such as octamethyl cyclotetrasiloxane, decamethyl cyclopentasiloxane, dodecamethyl cyclohexasiloxane, and tetradecamethyl cycloheptasiloxane; etc. may be mentioned.

The external composition of the present invention may contain, depending upon the need, the following other components as auxiliary components to an extent not detracting from the desired effect of the present invention.

For example, as the oil component, hydrocarbon oils such as liquid paraffin, isoliquid paraffin, squalane, oils and fats such as olive oil, palm oil, coconut oil, macadamia nut oil, jojoba oil; higher alcohols such as isostearyl alcohol; ester oils such as higher aliphatic oils and isopropyl myristate, etc. may be formulated in the external composition of the present invention. Among these oil components, in particular, formulating a polar oil in the external composition of the present invention enables improvement of the stability with the elapse of time.

Further, a benzophenon derivative, para-aminobenzoate derivative, para-methoxysuccinate derivative, salicylate derivative, and other UV absorbers; humectants, blood circulation promoters, refrigerants, antiperspirants, bactericides, skin activators, anti-inflammatory agents, vitamins, antioxidants, antioxidant adjuvants, preservatives, flavors and fragrances, etc. may be blended in the external composition of the present invention.

The cosmetic formulation of the present invention may be produced in an appropriate medium including but not limited to a paste, a powder, a cake, a crème, an oil, a lotion, a grease, a wax or similar cosmetic bases. The process to produce involves combining the cosmetic ingredients and desamino amino acid compound of any of formulas I-V preferably as the ester, amide or salt. The combination is mixed, kneaded, rolled, ground, heated or otherwise treated to form a substantially homogeneous mass or mixture for use. These steps can be accomplished by use of a kneader, grind wheel, rollers, mixer, heat exchanger, extruder and the like.

As explained above, the invention is exemplified by modification of the natural peptide neurotensin. In the following section, the gackground, modification and biological activity of neurotensin and the corresponding peptides of the invention are discussed.

### Neurotensin Structure and Biology

Neurotensin (NT) was first isolated from bovine hypothalami as a hypotensive peptide by Carraway and Leeman in 1973. Since then, NT has been shown to have numerous distinct physiological effects in the central nervous system (CNS) and the periphery. Hypothermia, antinociception, attenuation of d-amphetamine-induced hyperlocomotion, and potentiation of barbiturate-induced sedation are promoted by direct injection of NT into the brain. Peripherally, NT acts as a hormone to induce hypotension and decrease gastric acid secretion. Structurally, NT is a linear tridecapeptide with the following sequence: pGlu-Leu-Tyr-Glu-Asn-Lys-Pro-Arg-Arg-Pro-Tyr-Ile-Leu-OH. Early in the history of NT research, it was shown that the C-terminal hexapeptide fragment, Arg⁸-Arg⁹-Pro¹⁰-Tyr¹¹-Ile¹²-Leu¹³ [NT(8-13)], was equipotent at producing the physiological effects of NT in vitro and in vivo.

Tanaka and colleagues first identified an NT receptor (NTR₁) from rat brain in 1990. Since then, human NTR₁ has been successfully cloned and expressed. Both are classic G-protein coupled receptors containing seven transmembrane (7TM) domains and share 84% homology. Second messenger systems, including cGMP production, calcium mobilization, and phosphatidylinositol turnover, are triggered upon NTR₁ activation. The mRNA for NTR₁ is expressed in both rat and human brain and intestine. A second NT receptor (NTR₂) with a substantially lower affinity for NT than NTR₁, (k_{d}≅2.5 and 0.5 nM respectively), also has been identified in rat and human brain (23-25). NTR₂ is also a 7TM/G-protein coupled receptor, yet has a shorter N-terminal extracellular tail and a longer third intracytoplasmic loop compared to NTR₁. A third receptor (NTR₃) was cloned from a human brain cDNA library and found to be identical to the previously cloned gp95/sortilin. NTR₃ is a non-G-protein coupled sorting protein having only a single transmembrane region.

### NT as an Endogenous Neuroleptic

Several distinct lines of evidence implicate NT in the pathophysiology of schizophrenia. Advances in the dopamine theory of schizophrenia support that a flaw in the convergence of various neural circuits on the mesolimbic dopamine system is responsible for the development of the disorder. The anatomical positioning of the NT system is such that it interacts with the glutaminergic, dopaminergic, GABAergic, and serotonergic systems within the brain. In particular the NT and dopamine systems are closely related within the nucleus accumbens, the area of the brain believed to be responsible for delusions and hallucinations. NTR₁ receptors are dense in the ventral tegmental area, a brain region closely associated with the neuronal systems described above. Almost 90% of NT receptors are located on dopaminergic neurons and over 80% of dopamine neurons in the brain express NTR₁. Co-localization of the NT system with brain regions implicated in schizophrenia also imply its involvement.

### Neurotensin and its Biological Activity

Since NT was hypothesized as an “endogenous neuroleptic” and NT(8-13) was identified as its active fragment, efforts have been made to develop NT(8-13) derivatives as potential antipsychotics. Two groups in particular, the Eisai pharmaceutical company (Tokyo, Japan) and the Richelson research group (Mayo Clinic, Jacksonville, Fla.) have prepared numerous derivatives of NT(8-13) analogues that showed promise as antipsychotic drugs. In particular, amino acid substitutions at Arg⁸, Arg⁹, Tyr¹¹, and Ile¹² have yielded several analogues that are centrally active after peripheral administration.

An Eisai compound (the Eisai hexapeptide) was the first NT(8-13) analogue that elicited behavioral effects after peripheral administration. However, the various modifications incorporated in this peptide resulted in a 700-fold loss of binding affinity at NTR₁. In addition, this analogue was not able to elicit central activity after oral administration.

More recently, NT69L has been developed by the Richelson group as an NT(8-13) analogue that maintains nanomolar binding affinity at NTR₁ (K_{d}=1.55 nM) (55) and exhibits a pronounced hypothermic effect after a 1 mg/kg injection (−5.3° C. at 90 min PI) (41). NT69L also attenuates hyperactivity induced by both cocaine and d-amphetamine. However, tolerance to its hypothermic effect and to its suppression of d-amphetamine induced hyperactivity was observed after chronic administration of the compound. As with the Eisai hexapeptide, NT69L produced only a slight hypothermic response after oral administration.

### EXAMPLES

### Example 1

### Summary of Neurological Effects of the NT Peptides of the Invention

The N-terminal alpha methyl, alpha desamino homolysyl and orinthyl analogues of NT(8-13) prepared according to the invention (see the foregoing general discussion and the Examples) were synthesized and screened for activity in numerous behavioral assays predictive of antipsychotic potential. These peptides induced hypothermia in a dose-dependent fashion after oral administration. In addition, oral administration of the peptides significantly reduced d-amphetamine induced hyperlocomotion, a measure of the therapeutic efficacy of current or potential APDs. The low dose of peptide (10 mg/kg) that elicits a significant response after oral administration in these assays is significant. The peptides also demonstrate an ability to maintain efficacy after repeated administration. In fact they demonstrate an ability to increase maximal hypothermic response over time, implying that repeated administration may actually improve their CNS activity. Thus, the NT peptides of the invention are shown to have biological activity like that of the known naturally occurring peptide NT and are more selective. Details of these effects are as follows.

Hypothermia as a Preliminary Screen of CNS Activity. NT induces hypothermia when directly administered into the CNS. As a result, induced hypothermia can be used to determine the ability of NT(8-13) peptides of the invention to cross the BBB after peripheral administration and indirectly to determine their in vivo CNS activity. The hypothermic effect of NT can be attributed to its actions at NTR₁, the NTR most often implicated in the pathophysiology of schizophrenia. An NT(8-13) peptide that induced hypothermia after IP injection is thus shown to be an antipsychotic agent. A significant hypothermic effect would-demonstrate that the peptide showed marked improvements in blood stability and membrane crossing.

IP injection is the standard route of administration to determine the extent of BBB crossing of neurotensin analogues. The methods and protocols are provided in the Examples section. IV administration results in a dose that is completely available to the systemic circulation. By contrast, an IP injection is a more rigorous test of stability because the peptide is exposed to first pass metabolism in the liver.

The hypothermic effects of peptides 28-30, after a 5 mg/kg IP injection, are given in Table 4. Each peptide exhibited a significant effect over a 5 hr time course. The hypothermic results for these three peptides demonstrate that the substitution of an alpha alkyl group in place of the N-terminal amine group (i.e., the α-methyl group) does not abolish the in vivo activity of the NT(8-13) peptide. For use as an antischizophrenic pharmaceutical, the ability of these peptides to elicit CNS activity after oral administration was evaluated.

TABLE 4
Amino acid sequence of NT(8-13) analogues.

| | Amino Acid Sequence | | | | | | | |
|---|---|---|---|---|---|---|---|---|
| Peptide | N-terminus | 8^{a} | 9 | 10 | 11 | 12 | 13 | C-terminus |
| KH28 | CH₃ | L-Hlys | L-Arg | L-Pro | L-Tyr | L-tLeu | L-Leu | COOH |
| (ABS201) | | | | | | | | |
| KH29 | CH₃ | 7 | L-Arg | L-Pro | L-Tyr | L-tLeu | L-Leu | COOH |
| KH30 | CH₃ | 9 | L-Arg | L-Pro | L-Tyr | L-tLeu | L-Leu | COOH |

TABLE 5
Hypothermic response to IP administration of NT(8-13) and NT(8-13)
analogues.

| Peptide^{a} | tₘₐₓ (min)^{b} | Δ in BT (° C.)^{c} |
|---|---|---|
| NT(8-13) | 90 | −0.45 ± 0.17^{d} |
| ABS201 | 150 | −2.51 ± 0.17 |
| KH29 | 150 | −3.75 ± 0.24 |
| KH30 | 300 | −3.84 ± 0.20 |

^{a}IP dose was 5 mg/kg for all peptides.
^{b}tₘₐₓ (min) = Time to maximal temperature decrease.
^{c}Δ in BT (° C.) = Decrease in body temperature measured at tₘₐₓ
^{d}N = 5 for all peptides.

Oral Administration. A goal in the development of NT(8-13) peptides as antischizophrenic pharmaceuticals is to determine their ability to exhibit CNS activity after oral administration. The known NT peptides, NT69L and the Eisai hexapeptide, fail in this respect to elicit central activity when given orally. Accordingly, the N-terminal methyl peptides 28-30 were tested for their ability to induce hypothermia after oral administration.

ABS201, an example of a peptide of the invention, demonstrated maximal hypothermic responses greater than 2° C. (Table 6) and its maximal hypothermic effect was equal to its hypothermic effect after IP dosing (FIG. 9), resulting in an approximate oral bioavailability of 25%. While peptides 29 and 30 also were orally active, their ratio of oral activity to IP activity was not as balanced as that of ABS201. The oral activity of ABS201 was an important factor to support it as a lead NT(8-13) analogue for further evaluation of anti psychotic potential.

TABLE 6
Hypothermic response to chronic IP administration of ABS201.

| Peptide^{a} | tₘₐₓ (min)^{b} | Δ in BT (° C.)^{c} |
|---|---|---|
| Saline^{d} | 180 | 0.70 ± 0.20 |
| Day 1 | 120 | 2.72 ± 0.24 |
| Day 2 | 90 | 2.85 ± 0.26 |
| Day 3 | 120 | 3.74 ± 0.13 |
| Day 4 | 120 | 3.71 ± 0.13 |
| Day 5 | 90 | 3.83 ± 0.24 |

^{a}IP dose was 5 mg/kg for all days.
^{b}tₘₐₓ (min) = Time to maximal temperature decrease.
^{c}Δ in BT (° C.) = Decrease in body temperature measured at tₘₐₓ
^{d}N = 5 for all days.

TABLE 7
Comparison of the maximal hypothermic effects of peptide
ABS201 after IP administration.

| | Peptide |
|---|---|
| Dose | ABS201^{a} |
| 0.1 mg/kg | −1.14 ± 0.21 |
| 0.5 mg/kg | −1.92 ± 0.12 |
| 1.0 mg/kg | −2.63 ± 0.21 |
| 5.0 mg/kg | −3.61 ± 0.22 |

^{a}Change in body temperature (° C.) is the maximal decrease recorded for each individual dose.

Schizophrenia Investigation. The blockade of locomotion caused by d-amphetamine, a “DA agonist”, has become the standard measure of therapeutic efficacy of current or potential drug candidates for treatment of schizophrenia. This model operates on the assumption that the direct stimulation of DA receptors within the mesolimbic DA system is responsible for the locomotor response.

Catalepsy, commonly defined as a state of tonic immobility in rodents, is regarded as analogous to (extrapyramidal side effects) EPSEs in humans. Consequently, catalepsy is a side effect to be avoided in a successful drug candidate. Concurrently, the degree to which a drug candidate causes catalepsy in rats may also be used as a predictor for the probable occurrence of EPSEs associated with that particular candidate.

Hypothermic Analysis. To examine the antipsychotic properties of ABS201, a dose-response curve for hypothermic induction after IP administration was generated. In addition, the hypothermic effects elicited by oral administration of ABS201 (10 mg/kg-30 mg/kg) were determined. The ability of ABS201 to reduce d-amphetamine induced hyperlocomotion after both IP and oral administration also was measured. To assess the effects on CNS activity caused by prolonged treatment of ABS201, hypothermia and attenuation of d-amphetamine induced hyperlocomotion were measured after repeated daily dosing. Finally, the bar test was utilized to measure catalepsy as a predictor of EPSEs in humans.

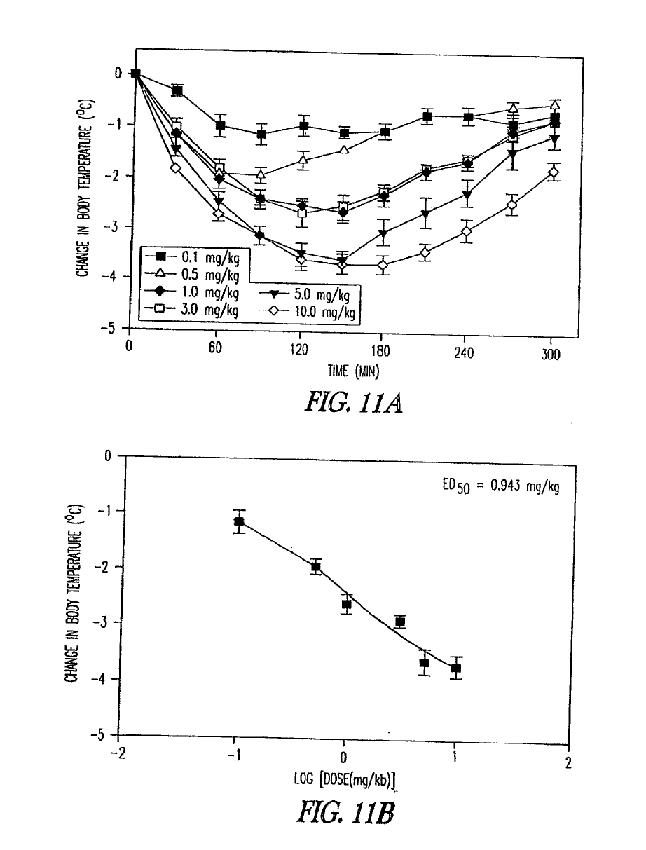

The dose-response curve for ABS201 after IP injection over a concentration range of 0.1-10 mg/kg (FIG. 11) gave some conflicting results. First, the maximal effect elicited at 5 mg/kg (−3.61±0.22° C. at 150 min PI) was a full degree greater than the maximal effect seen after the preliminary screen (−2.51±0.17° C. at 150 min PI). This discrepancy is most likely due to environmental factors (air temperature, rectal probes, rat size, etc.) that can affect the rats' response. Most importantly, ABS201 continued to elicit a significant CNS effect irrespective of these differences. The ED₅₀ value for ABS201, 0.943 mg/kg., compares favorably with other NT(8-13) analogues with CNS activity (41, 60).

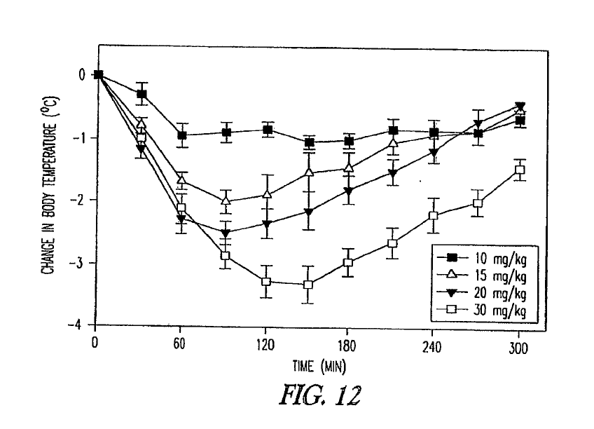

ABS201 also induced hypothermia in a dose-dependent fashion after oral administration (FIG. 12). A significant hypothermic effect was demonstrated at 10 mg/kg, the lowest dose tested (−1.02±0.10° C. at 150 min PI). The generation of an ED₅₀ value for the oral administration of ABS201 was impractical due to the inordinate amount of peptide necessary to produce a complete dose-response curve. Previous NT(8-13) analogues that have been under development as antipsychotic compounds have contained a Trp¹¹ substitution. Evidence from the studies presented herein supports the theory that this modification abolishes the oral activity of a NT analogue. Further studies are necessary to determine what specific role Tyr¹¹ plays in the oral bioavailability of NT(8-13) analogues.

The blockade of locomotion caused by d-amphetamine, a “DA agonist”, has become the standard measure of therapeutic efficacy of current or potential schizophrenia drug candidates, and NT(8-13) analogues currently under investigation as candidates have demonstrated the ability to decrease d-amphetamine induced hyperactivity in a dose-dependent fashion. Sound- and light-attenuated locomotor cages are used to measure the ability of potential candidates to decrease d-amphetamine-induced hyperactivity.

The effects of ABS201 on d-amphetamine induced hyperactivity at varying doses were also examined. ABS201 significantly reduced hyperlocomotion for all doses tested (doses of 3 mg/kg and 10 mg/kg not shown). Another hallmark of current APDs is the ability to reduce spontaneous locomotor activity. All ABS201 dose groups responded significantly lower than saline during the drug phase, indicating the ability of ABS201 to reduce spontaneous activity.

The ability to attenuate d-amphetamine induced hyperlocomotion after oral administration is also demonstrated by ABS201. During the drug phase, only the 10 and 30 mg/kg doses reduced spontaneous locomotor activity. The lack of significance seen with the 20 mg/kg dose is most likely an anomaly resulting from slight variation in response for this group of rats. However, no main effect of DOSE was detected during the baseline phase, indicating that there is not a significant difference in baseline activity across the different dosing groups.

ABS201 maintained a significant CNS effect after repeated daily dosing (Table 6) and over the 5-day period the absolute hypothermic response increased. A comparison of the induced hypothermia of ABS201 on days 1 and 5 was made. On day 5, the maximal hypothermic response was achieved faster (90 min) compared to day 1 (120 min). In contrast to day 1, on day 5 the maximal hypothermic effect was not maintained for an extended period, implying that while repeated dosing does not decrease the maximal effect, it may reduce the duration of the hypothermic effect. Repeated daily dosing had no effect on the ability of ABS201 to attenuate d-amphetamine induced hyperlocomotion. Both the acute and chronic dosing groups produced a reduction in hyperactivity that was significant for almost two hours after amphetamine administration. Of note, chronic administration of ABS201 did abolish its inhibitory effect on spontaneous locomotor activity.

Cataleptic Analysis. In laboratory tests, catalepsy is characterized by the inability of an animal to correct its position after placement in an unnatural posture. Catalepsy tests can be greatly influenced by a number of variables. These include stress-induced inhibition of catalepsy caused by a new environment and the contribution of learned “pseudo-catalepsy” that can result upon repeated measures with the same animal. To circumvent these potential confounding factors, tests are performed on an animal only once in a quiet, controlled environment.

Neither ABS201 (5 mg/kg) nor saline caused catalepsy after peripheral administration. Haloperidol, a typical antischizophrenic drug known to produce a fully cataleptic response in rats, induced catalepsy that lasted for greater than 30 sec. These results demonstrate that ABS201 does not induce catalepsy after peripheral administration, a hallmark of current clinically effective candidates.

Bioavailability Study with CACO-2 Cells. Caco-2 cells, derived from a human colorectal carcinoma, spontaneously differentiate into polarized cells that exhibit well-developed microvilli and brush-border enzymes. These features make the cells an excellent model of the human small intestine. A strong correlation between uptake of a compound in the Caco-2 cell model and oral bioavailability of the compound has been identified.

ABS201 is stable in rat serum for greater than 24 hours, however, its stability in cells has not been determined. Consequently, determination of the ability of intact peptide to enter the Caco-2 cells in the uptake experiments will show oral bioavailability and cellular stability. Reverse phase HPLC is an ideal method to analyze the solubilized cell components for ABS201 and ABS201 degradation products. This analysis will show oral availability and cellular stability. Fractions can be collected at determined intervals and counted for radioactivity via LSC. By establishing the ABS201 elution time via a standard gradient, direct comparisons can be made to the contents of Caco-2 cells after uptake experiments.

To verify that intact peptide is entering the Caco-2 cells and in a preliminary attempt to assess the stability of the peptides in cell culture, a RP-HPLC assay to analyze ABS201 after cellular uptake can be carried out. After a 2 min incubation, intact ABS201 likely can be identified within the cells using HPLC techniques. These studies will demonstrate that ABS201 can be extensively taken up by the Caco-2 cells thus showing its oral bioavailability.

### Example 2

### Compound Synthesis

The following examples and protocols are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and is at room temperature, and pressure is at or near atmospheric.

Starting Materials. Solvents are from Fisher Scientific (Pittsburgh, Pa.) and reagents from Aldrich (Milwaukee, Wis.) unless otherwise noted. Abbreviations. Trisyl-N₃, 2,4,6-triisopropylbenzenesulfonyl azide; Et₃N, triethylamine; t-BuCOCl, trimethylacetylchloride; n-BuLi, n-butyl lithium; H₂, hydrogen gas; Pd—C, palladium on activated carbon; Xps, (S)-(−)-4-benzyl-2-oxazolidinone; KHMDS, potassium bis(trimethylsilyl) amide; CH₃I, methyl iodide; H₂O₂, hydrogen peroxide; LiOH, lithium hydroxide; THF, tetrahydrofuran; CH₂Cl₂, dichloromethane; MgSO₄, magnesium sulfate; Hex, hexane; EtOAc, ethyl acetate; NaHCO₃, sodium bicarbonate; HCl, hydrochloric acid; N₂, nitrogen; H₂O, distilled water.

(3(2S),4S)-3-(2-methyl-5-bromo-1-oxovaleryl)-4-(phenylmethyl)-2-oxazolidinone (24a) (FIG. 3). Intermediate 23a was prepared as described previously (57). A solution of 17.4 mL (5 eq) of potassium bis(trimethylsilyl) amide (KHMDS) was added to 100 mL anhydrous tetrahydrofuran (THF) and cooled to −78° C. under positive nitrogen (N₂) pressure. A solution of 23a (5.18 g, 15.23 mmol) in 10 mL THF under N₂ was cooled to −78° C. and cannulated into the KHMDS solution. This mixture was stirred at −78° C. for 30 min to effect enolate formation. Methyl iodide (CH₃I) (1.90 mL, 2 eq) was added to the solution via cannula and stirred at −78° C. for 1 hr at which time the reaction was quenched with 4.09 mL (5 eq) of glacial acetic acid. The solution was warmed to room temperature while stirring over 2 hr and the THF removed in vacuo. The resulting yellow slurry was dissolved in 200 mL half-saturated brine and extracted with CH₂Cl₂ (3×100 mL). The CH₂Cl₂ layers were combined, dried over anhydrous magnesium sulfate (MgSO₄), filtered, and evaporated in vacuo to yield a yellow oil. The crude oil was purified over silica gel eluting with 3:1 hexane:ethyl acetate (Hex:EtOAC) to give 2.81 g (52% yield) of pure 26a. ¹H NMR (400 MHz, CDCl₃) δ 7.38-7.15 (m, 5H), 4.71-4.63 (m, 1H), 4.18-4.15 (d, J=5.0 Hz, 2H), 3.71-3.65 (m, 1H), 3.41-3.33 (m, 2H), 3.27-3.20 (dd, J=4.0, 13.8 Hz, 1H), 2.89-2.81 (dd, J=10.0, 14.2 Hz, 1H), 1.90-1.55 (m, 4H), 1.24-1.20 (d, J=7.4, 3H); ¹³C NMR (100 MHz, CDCl₃) δ 176.8, 153.2, 135.2, 129.6, 129.1, 127.6, 66.4, 55.6, 38.3, 37.6, 33.8, 32.2, 31.8, 17.9.

(3(2S),4S)-3-(2-methyl-6-bromo-1-oxohexanyl)-4-(phenylmethyl)-2-oxazolidinone (24b). A slightly modified procedure was used to give 24b. Directly following KHMDS addition to 23b, 5 eq of CH₃I was added and the reaction stirred at −78° C. under N₂ for 1 hr. Quenching with glacial acetic acid and subsequent extraction and purification protocol was as described above for 24a. Additional silica gel purification eluting with 100% CH₂Cl₂ gave pure 24b in 10% yield. ¹H NMR (400 MHz, CDCl₃) δ 7.36-7.19 (m, 5H), 4.72-4.65 (m, 1H), 4.25-4.16 (d J=4.2 Hz, 2H), 3.77-3.67 (m, 1H), 3.46-3.36 (t, J=7.0 Hz, 2H), 3.29-3.22 (dd, J=4.0, 14.0 Hz, 1H), 2.82-2.74 (dd, J=9.0, 14.0 Hz, 1H), 1.92-1.74 (m, 3H), 1.50-1.42 (m, 3H), 1.25-1.21 (d, J=7.2 Hz, 3H); ¹³C NMR (100 MHz, CDCl₃) δ 176.9, 153.2, 135.3, 129.6, 129.1, 66.4, 55.6, 38.1, 37.8, 34.1, 32.8, 32.5, 26.1, 18.7.

(3(2S),4S)-3-(2-methyl-7-bromo-1-oxoheptyl)-4-(phenylmethyl)-2-oxazolidinone (24c). Compound 24c was produced in 56% yield from 23c following the procedure outlined for compound 26a. ¹H NMR (400 MHz, CDCl₃) δ 7.41-7.22 (m, 5H), 4.74-4.66 (m, 1H), 4.25-4.19 (d, J=4.0 Hz, 2H), 3.77-3.70 (m, 1H), 3.45-3.39 (t, J=7.0 Hz, 2H), 3.31-3.23 (dd, J=3.7, 13.7 Hz, 1H), 2.84-2.77 (dd, J=10.0, 12.5 Hz, 1H), 1.91-1.77 (m, 3H), 1.50-1.32 (m, 5H), 1.25-1.20 (d, J=7.0 Hz, 3H); ¹³C NMR (100 MHz, CDCl₃) δ 177.2, 153.1, 135.4, 129.7, 129.1, 127.5, 66.4, 55.7, 38.2, 37.9, 34.3, 33.4, 32.8, 28.4, 27.7, 17.8.

2(S)-Methyl-5-bromovaleric acid (25a). A solution of 24a (10.41 g, 29.4 mmol) in 100 mL THF and 40 mL H₂O was cooled to 0° C. while stirring. To this solution was added 12.12 mL (3.5 eq) 30% hydrogen peroxide (H₂O₂) followed by 2.41 g (2 eq) lithium hydroxide (LiOH) and the solution was stirred at 0° C. for 50 min. After 50 min, 94 mL sodium sulfite (0.183 g/mL H₂O) and 288 mL 0.5N sodium bicarbonate (NaHCO₃) were added. The THF was removed in vacuo and the remaining aqueous solution extracted with CH₂Cl₂ (3×100 mL). The aqueous layer was acidified to pH 2 with 25% HCl and extracted with EtOAc (3×100 mL). The EtOAc fractions were combined and concentrated in vacuo to give 4.01 g (70% yield) of 27a as a pale oil. ¹H NMR (400 MHz, CDCl₃) δ 3.46-3.38 (t, J=6.0 Hz, 2H), 2.56-2.46 (m, 1H), 1.95-1.60 (m, 4H), 1.25-1.20 (d, J=7.0 Hz, 3H); ¹³C NMR (100 MHz, CDCl₃) δ 183.1, 38.9, 33.6, 32.1, 30.5, 17.3.

2(S)-Methyl-6-bromohexanoic acid (25b). Compound 25b was produced in 77% yield from 24b following the procedure outlined for 25a. NMR (400 MHz CDCl₃) δ 3.45-3.38 (t, J=6.2 Hz, 2H), 2.55-2.45 (m, 1H), 1.92-1.85 (m, 2H), 1.77-1.68 (m, 1H), 1.55-1.46 (m, 3H), 1.24-1.19 (d, J=7.8 Hz, 3H); ¹³C NMR (100 MHz, CDCl₃) δ 183.5, 39.5, 33.8, 32.9, 32.7, 26.0, 17.2.

2(S)-Methyl-7-bromoheptanoic acid (25c). Compound 25c was produced in 74% yield from 24c following the procedure outlined for 25. ¹H NMR (400 MHz, CDCl₃) δ 3.43-3.36 (t, J=6.8 Hz, 2H), 2.51-2.42 (m, 1H), 1.90-1.64 (m, 3H), 1.49-1.32 (m, 5H) 1.20-1.14 (d, J=7.0 Hz, 3H); ¹³C NMR (100 MHz, CDCl₃) δ 183.6, 39.5, 34.1, 33.5, 32.7, 28.2, 26.6, 17.1.

### Example 3

### Alpha Methyl, Alpha Desamino, Omega N-Substituted Homolysyl and Orinthyl (8) Neurotensin (8-13)

Alpha methyl, alpha desamino omega N-substituted homo lysyl and orinthyl (8) neurotensin (8-13) were synthesized (FIG. 7). The α-methyl bromo acids, 27a and c, were coupled to the resin-bound peptide as outlined in the general section. The solid state coupling was conducted as follows.

Resin bound N alpha Fmoc leucine was swelled in DMF prior to Fmoc cleavage with piperidine (20% in DMF). The piperidine solution was removed with vacuum filtration and the resin-bound amino acid washed with DMS and methylene chloride (5× each). Amino acids (4 eq) were activated in DMF with HOBt (4 eq) PyBOP ((4 eq) and DIPEA (10 eq) and added directly to the peptide reaction vessel. Amino acids were coupled for 6 hours, the resins was washed with DMF and methylene chloride and monitored with a Kaiser test for the presence of free amines. Residues were recoupled when necessary. This procedure was repeated with subsequent amino acids to give the penultimate peptide sequence (pentamer).

Aliquots of the resin-bound pentamer were then coupled with the appropriate omega bromo carboxylic acid as described above to give the N-omega bromo acyl pentamers. The N-omega acyl pentamers were then reacted with ammonia, dimethyl amine or trimethyl amine as described in these Examples to produce the desired peptides of the invention. Acid catalyzed deprotection of the side chain protecting groups was performed with a TFA solution containing appropriate scavengers.

RP-HPLC purification using a linear gradient of 15% to 75% B over 55 min at a constant flow rate of 4 mL/min afforded pure omega bromo peptides 53 and 54. These bromo peptides were reacted at 40° C. for 12 hr with 150 eq of ammonium hydroxide (29% in H₂O), methylamine (40% in H₂O), dimethylamine (40% in H₂O), or trimethylamine (40% in H₂O) in ethanol (EtOH). Solvents were removed in vacuo and crude peptides were taken up in mobile phase and purified with a linear gradient of 2% to 50% B over 65 min at a constant flow rate of 4 mL/min.

Peptides were characterized and assessed for purity via MALDI-TOFMS on a Voyager DE-STR System 4117 mass spectrometer (Applied Biosystems, Foster City, Calif.). Peptides were used at greater than 95% purity in vivo.

### Example 4

### Identification of Antipsychotic NT Peptide Analogs

Structures of Compounds. The peptide analogs evaluated contain one non-natural amino acid (Scheme 1) or desaminoacid (Scheme 2).

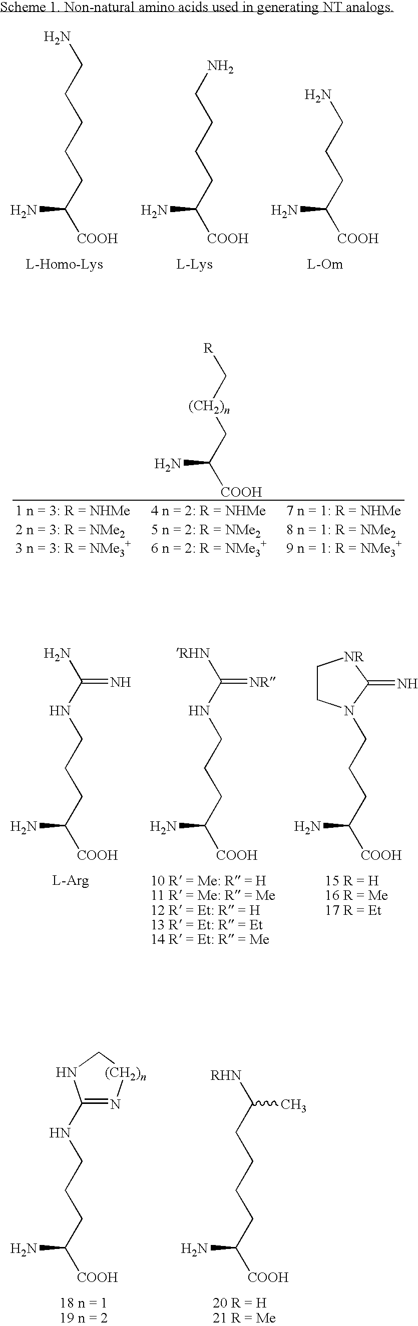

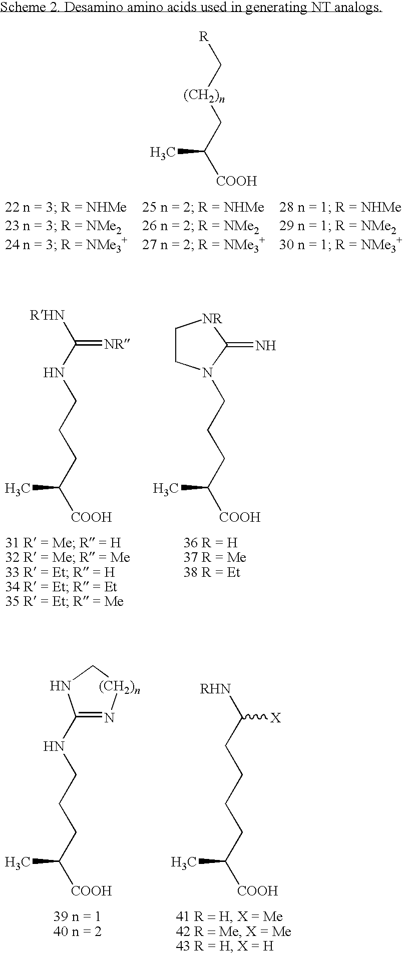

Benchmarking Compounds. From an initial screen of about 50 compounds, those listed in Table 8 were selected for further testing and development as antipsychotic compounds. These compounds are based on the neurotensin fragment NT(8-13). These compounds possessed useful characteristics as each: bound as competitive agonists in vitro to NTR-1, the brain neurotensin receptor involved in schizophrenia, demonstrated central activity when injected IP in rat using hypothermia as a surrogate for central activity, which also occurs through NTR-1 binding and elicited the appropriate activities in rat behavioral models of schizophrenia.

TABLE 8
Structures of NT[8-13]-based Peptides.

| Peptide # | Structure¹ |
|---|---|
| NT(8-13) | NH₂-Arg-Arg-Pro-Tyr-Ile-Leu-COOH |
| ABS13 | N₃-L-homolysine-Arg-Pro-Tyr-tertLeu-Leu-COOH |
| ABS41 | N₃-13-Arg-Pro-Tyr-tertLeu-Leu-COOH |
| ABS44 | N₃-7-Arg-Pro-Tyr-tertLeu-Leu-COOH |
| ABS46 | N₃-9-Arg-Pro-Tyr-tertLeu-Leu-COOH |
| ABS201 | 43-Arg-Pro-Tyr-tertLeu-Leu-COOH |
| ABS202 | 28-Arg-Pro-Tyr-tertLeu-Leu-COOH |
| ABS203 | 29-Arg-Pro-Tyr-tertLeu-Leu-COOH² |

¹Bolded numbers within peptide structures refer to the non-natural Arg and Lys residues shown in Schemes 1 and 2.

To further characterize the compounds, hypothermia induction (NTR-1 receptor binding) activity was evaluated with both oral and IP dosing of each compound. As seen in Table 9, all of the compounds except for ABS201 exhibited <10% oral activity. Interestingly, ABS201 had a 300% increase in oral activity over the previous most active compound. In addition, ABS201 achieved a faster response when administered orally versus IP. This is unique among the NT(8-13) derivatives.

TABLE 9
Hypothermic effects of NT(8-13) analogues after IP and oral
administration.

| | IP Dose^{a} | | | | |
|---|---|---|---|---|---|
| | | Δ in BT^{d} | Oral Dose^{b} | | App. |
| Peptide | tₘₐₓ^{c} (min) | (° C.) | tₘₐₓ^{c} (min) | Δ in BT^{d} (° C.) | Oral |
| Saline | 240 | −0.60± | 180 | −0.64± | NA |
| ABS13 | 150 | −4.26± | 90 | −1.66± | |
| ABS31 | 180 | −6.87± | 150 | −1.05± | NA |
| ABS44 | 150 | −5.07± | 150 | −1.58± | |
| ABS46 | 180 | −4.68± | 180 | −2.03± | |
| ABS201 | 150 | −2.51± | 90 | −2.49± | |
| ABS202 | 150 | −3.75± | 120 | −1.09± | NA |
| ABS203 | 300 | −3.84± | 150 | −1.30± | NA |

^{a}IP dose was 5 mg/kg for all peptides.
^{b}Oral dose was 20 mg/kg for all peptides.
^{c}tₘₐₓ (min) = Time to maximal temperature decrease.
^{d}Δ in BT (° C.) = Decrease in body temperature measured at tₘₐₓ.
^{e}Denotes a significant response (p < 0.05).
^{f}Approximate oral bioavailability was calculated from the relative areas under the hypothermia curve for each dosing regimen, corrected for amount of compound administered.
^{g}NA = none apparent (as the oral dosing was not significant over baseline).

### Example 5

### Protocols for Neurotesting of ABS201

General Animal Protocols. Male Sprague Dawley Rats (250-350 g) or Brattleboro rats (270-310 g) were obtained from Harlan (Indianapolis, 1N) and housed in an AAALAC-approved colony room maintained at a constant temperature and humidity. Lighting was controlled on a 12 hr light:dark cycle with lights on at 0700 hr. Animals were housed two per cage and fed laboratory chow and water ad libitum. All experiments were performed during the light cycle.

Animal Restraint. Rats were restrained in Plas-Labs® plastic cages fitted with wooden dowels to restrict movement. Rectal temperature probes (Physitemp®, RET-2, Clifton, N.J.), lubricated with mineral oil, were inserted into the rectum of each animal. Probes were connected to a microprobe thermometer (Physitemp®, BAT-12) in conjunction with a thermocouple selector (Physitemp®, SWT-5). Rats were allowed to acclimate to the cages for 1 hr prior to IP injection.

Peptide Preparation. Each peptide tested was dissolved in 0.9% NaCl to the appropriate final concentration for I.P. injection (10, 30 or 100 mg NT[8-13] or equimolar equivalent of analog in 1 mL 0.9% NaCl). For oral administration, the test article was administered directly into the animals' stomach by gavaging. Controls involved the administration of 0.9% NaCl. Thus, peptides were dissolved in saline, and following the equilibration period, rats were given an IP injection of peptide (5 mg/kg) or saline (1 mL/kg). Initial temperature values were the average temperatures of the rats immediately before and after the injection. Subsequent measurements were taken every 30 min for 5 hr. Statistical analysis was performed using a one-way analysis of variance (ANOVA) followed by Tukey's post hoc test for multiple comparisons using GraphPad Prism® to measure significance. Results were considered significant for p<0.05.

Hypothermia. Hypothermia induction is an easy and direct measure of neurotensin derivatives entering the brain and binding to NTR-1, the receptor involved in psychosis. For these experiments, animals were housed in individual Plexiglas cages without bedding at 25° C. For each rat, a thermistor probe was placed 4.5 cm into the rectum; baseline temperatures were recorded for 1 hr to allow habituation to the cages prior to injection of compound.

Dose-response curves for hypothermic induction. All animal restraint and hypothermia protocols were as described above. Variable slope dose-response curve and ED₅₀ value was generated using GraphPad Prism®.

d-Amphetamine induced hyperlocomotion. Experimentally I male Sprague-Dawley rats were housed as described above. Rats were handled for three days prior to testing to minimize experimenter induced hyperlocomotion on test day. For experiments, sound- and light-attenuated automated photocell beam activity chambers (AccuScan Instruments, Inc., Columbus, Ohio) were used to measure locomotion. Cages were connected to a VersaMax Analyzer (AccuScan) in conjunction with an IBM computer using VersaMax 1.80-0146 software (AccuScan) to record vertical and horizontal activity. Total activity values recorded were the sum of vertical and horizontal activity.

Each animal's activity was measured by how many times per minute the animal “trips” a photobeam in a computer-controlled photocell activity chamber (San Diego Instruments, San Diego, Calif.). The chambers were arranged in a four-level rack in a quiet room with ambient light and temperature approximately the same as in the separate housing room. The experiment was done over 6 days, with 1 run each day in mid-afternoon. On each day, 6 naïve rats were assigned to each treatment condition. Doses were assigned so that each was assessed in 12 chambers that were distributed over the apparatus, which controls for variations in lighting, etc. of the different chambers.

Rats were placed in the activity chambers for 30 minutes to habituate and establish baseline activity levels. Then, rats were removed and given an IP or oral dose of peptide (N=7) or saline (N=8) and returned to the chamber to establish the peptide's effect on spontaneous activity levels. About 30 minutes after administration, rats were removed and given an IP injection of d-amphetamine (1 mg/kg) or vehicle and returned to the chamber for a further 1 hr to assess the effect of the peptide on induced hyperlocomotion.

Chronic Testing Protocols. For chronic hypothermia testing, rats were given an IP injection of ABS201 (5 mg/kg) or saline once daily for five consecutive days. Induced hypothermia was monitored and tested for significance as described above. To assess the ability of ABS201 to decrease d-amphetamine induced hyperactivity after repeated administration, rats were divided into three dosing groups; chronic, acute, and control (N=7 for all groups). On days 1-4, the chronic group received an IP injection of ABS201 (5 mg/kg) while the acute and control groups received saline. On test day, day five, chronic and acute animals received ABS201 (5 mg/kg) while control animals received saline. The test protocol for day five was as described above.

Catalepsy Assessment. ABS201 (5 mg/kg) was dissolved in saline (1 ml/kg). Rats were given an IP injection of peptide (5 mg/Kg), saline, or haloperidol (1 mg/kg). After 3 hours, catalepsy was measured using the horizontal bar test. Briefly, the front paws were placed directly on a horizontal bar 5 mm in diameter placed 7.5 cm above the cage floor. The rat was held in this position for 3 sec and then released. The time from release until the paws return to the cage floor was measured and recorded. A cut-off time of 30 sec was observed; this indicated a fully cataleptic animal as cataleptic animals are frozen to the bar. Normal animals withdraw virtually immediately. Measures were repeated every 30 min for 4 hr. Data are means±SEM (p<0.01).

### Example 6

### Results of Neurotesting of ABS201

Hypothermic induction by ABS201 through IP and PO dosing. ABS201 has an ED₅₀ of about 1 mg/kg when administered IP, and 10 mg/kg administered orally (hypothermia data not shown, see amphetamine-induced hyperlocomotion results below). As seen in FIG. 9, ABS201 actually induces a quicker hypothermic effect when dosed orally versus IP at 2× the approximate ED₅₀ for each compound.

Dose-response curves. The dose-response curve for ABS201 after IP injection over a concentration range of 0.1-10.0 mg/kg is shown in FIG. 11. The calculated ED₅₀, value is 0.943 mg/kg. The hypothermic response to the oral administration of ABS201 over a concentration range of 10.0-30.0 mg/kg is shown in FIG. 12.

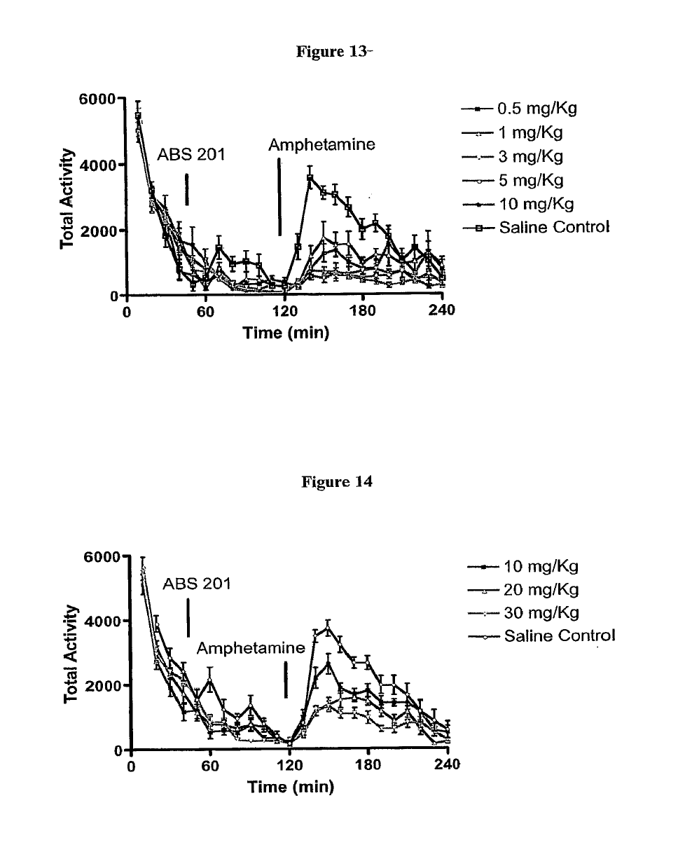

Attenuation of d-amphetamine induced hyperlocomotion after IP and oral dosing. FIG. 13 illustrates that ABS201 delivered IP inhibits amphetamine-induced hyperlocomotion with an ED₅₀ of about 1 mg/kg. FIG. 14 illustrates that ABS201 delivered by oral dosing induced hyperlocomotion with an approximate ED₅₀ of about 10 mg/kg. Larger doses were not attempted in the PO administration experiments due to lack of sufficient material; however, the IP data clearly illustrate that the hyperlocomotion effect can be fully reversed.

Separate two-factor ANOVAs for DOSE X TIME were performed for each different time-phase of the assay. Phases for the IP dosing and oral dosing experiments consisted of habituation (time points 10-60), drug (time points 70-120), and amphetamine (time points 130-240).

For IP dosing experiments, during the habituation phase, there was a main effect of TIME [F(5,185)=264.335 (p<0.001), indicating a gradual decrease in activity levels over time regardless of dose. Tukey's post-hoc tests, collapsed over dose, indicated activity levels for time points 10-30 were significantly higher than time points 40-60 (p<0.001). These results are attributed to the initial spontaneous exploratory activity associated with the novel environment. During the drug phase, there was a main effect of TIME [F(5,185=12.336 (p<0.001)] and a main effect of DOSE [F(5.37)=11.775 (p<0.001)]. The main effect of TIME resulted from a decrease in activity for all doses relative to the first time point (70 min). Tukey's post-hoc tests, collapsed over time, indicated that all doses responded significantly different from saline (p<0.001). During the amphetamine phase, there was DOSE X TIME interaction [F(55,407)=4.474 (p<0.001)]. Tukey's post-hoc tests revealed that all ABS201 dose groups demonstrated reduced locomotor activity for time points 130-200 as compared to saline (p<0.05).

For oral dosing experiments, during the habituation phase, there was a main effect of TIME [F(5,120)=201.979 (p<0.001)], indicating a gradual decrease in activity levels over time, regardless of dose. Tukey's post-hoc tests, collapsed over dose, indicated activity levels significantly decreased at each time point. During the drug phase, there was DOSE X TIME interaction [F(15,120)=11.584 (p<0.037)]. Tukey's post-hoc tests, collapsed over time, indicated that only the 10 mg/kg and 30 mg/kg dose groups responded significantly different from saline (p<0.01). During the amphetamine phase, there was DOSE X TIME interaction [F(11,264)=35.616 (p<0.001)]. Tukey's post-hoc tests revealed that all dose groups demonstrated reduced locomotor activity for time points 140-180 as compared to saline (p<0.05). In addition, the 20 mg/kg and 30 mg/kg dose groups responded significantly lower than the saline group at time points 190-200.

Effects of chronic ABS201 administration of hypothermic induction. ABS201 maintained a significant CNS effect after repeated daily dosing (Table 6) and over the 5-day period the absolute hypothermic response increased.

Effects of repeated ABS201 dosing on d-amphetamine induced hyperactivity. Separate two-factor ANOVAs for GROUP X TIME were performed for each different time-phase of the experiment. Phases were consistent with those described above. During the habituation phase, there was a main effect of TIME [F(5.90)=146.164 (p<0.001)], indicating that there was a gradual decrease in activity levels over time, regardless of group. Tukey's post-hoc tests, collapsed over dose, indicated activity levels for time points 10-20 were significantly higher than time points 30-60 (p<0.001). These results are attributed to the habituation of the rats to a novel environment over time. During the drug phase, there was a main effect of TIME [F(5.90)=13.512 (p<0.001)] and a main effect of GROUP [F(2.18)=4.37 (p=0.028)]. The main effect of TIME resulted from a decrease in activity for all doses relative to the first time point (70 min). Tukey's post-hoc tests, collapsed over time, indicated that only the acute group responded significantly different from saline during the drug phase (p<0.05). During the amphetamine phase, there was GROUP X TIME interaction [F(22.198)=4.069 (p<0.001)]. Tukey's post-hoc tests revealed that both the acute and chronic groups demonstrated reduced locomotor activity for time points 140-220 as compared to saline (p<0.05).

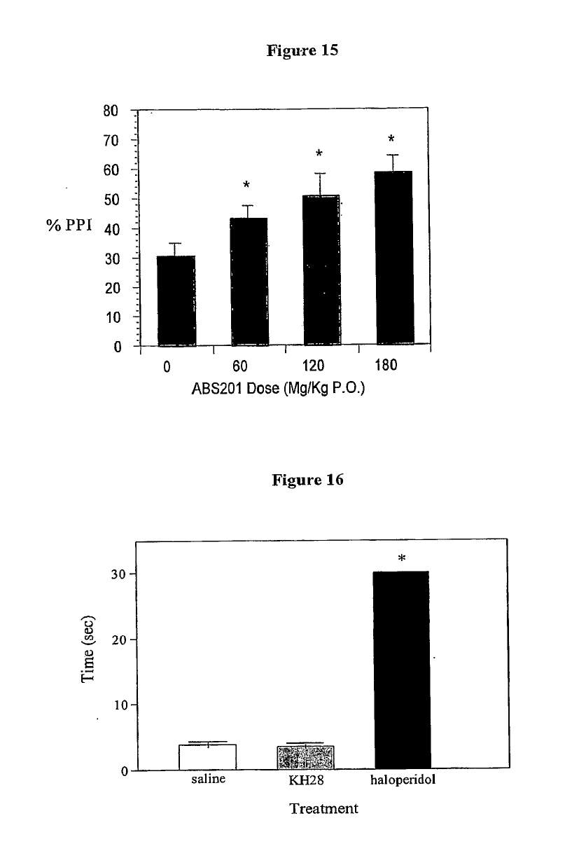

Reversal of Prepulse Inhibition. Prepulse inhibition (PPI) of acoustic startle is decreased in unmedicated schizophrenia patients and similar deficits can be induced in rats through pharmacological, environmental, or neuroanatomical manipulations. Recently, it was reported that Brattleboro (BB) rats, a Long Evans (LE) strain with a single gene mutation, have inherent deficits in PPI homologous to those observed in schizophrenia patients (see Feifel et al. Neuropsychopharmacology 29:731 (2004)). Atypical-type antipsychotics are active in this model. As seen in FIG. 15, orally dosed ABS201 reverses PPI in Brattleboro rats.

Catalepsy assessment. Neither ABS201 (5 mg/kg) nor saline caused catalepsy after peripheral administration (N=5). Haloperidol, a typical APD known to produce a fully cataleptic response in rats, induced catalepsy that lasted for greater than 30 sec. See FIG. 16.

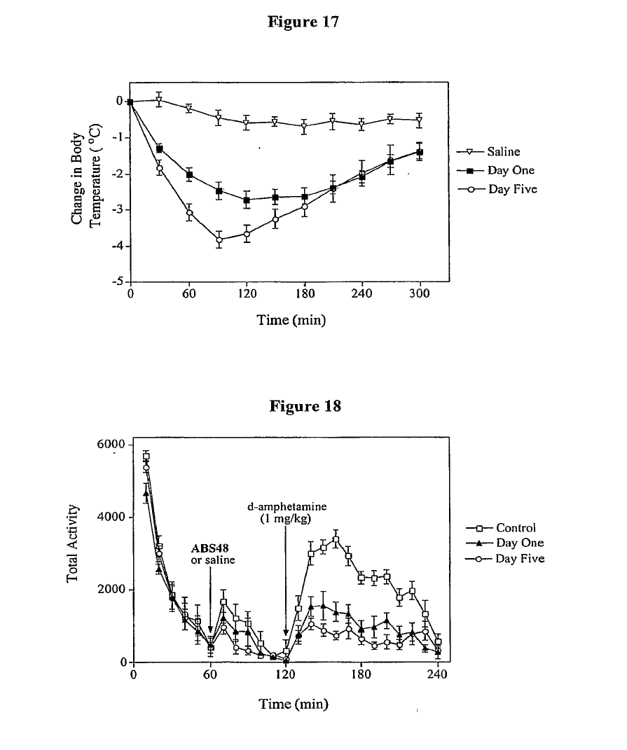

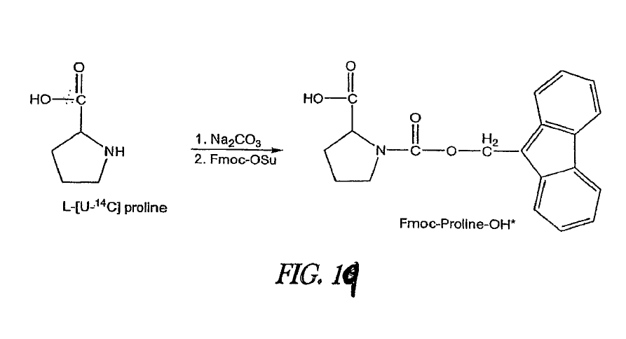

Dose Tolerance. The animals were I.P. dosed with ABS201 for 5 days straight at 5× the ED50, using both induction of hypothermia and inhibition of amphetamine-induced locomotion as the monitoring methods using protocols described above. Dose tolerance is not observed with either compound (see FIGS. 17 and 18).

Summary of the Behavioral Effects of ABS201. The “gold standard” animal model for evaluating a molecule with antipsychotic potential is inhibition of amphetamine-induced hyperlocomotion. ABS201 is active in a dose-dependent fashion whether IP or orally injected (FIGS. 13 and 14). The action of ABS201 is apparent following both IV and PO administration; dose-dependent; and long acting (observable 1 hr post administration and apparent for at least one additional hour).

The effects of ABS201 and haloperidol on catalepsy was examined in rats. Rats (N=5) were given an IP injection of ABS201 (5 mg/kg) or haloperidol (1 mg/kg). After 2 h, catalepsy was measured using the horizontal bar test. Data are means+/−SEM (p<0.01). ABS201 does not induce a cataleptic state in rats (FIG. 16), is not antinociceptive, and tolerance to multiple dosings does not occur either with monitoring hypothermia or inhibition of amphetamine-induced hyperlocomotion (FIGS. 17 and 18). Thus, ABS201 reliably induces hypothermia in rodents following both IV and PO administration. The action of ABS201 is: dose-dependent; and long acting, being observable for a period of 3-4 hours following administration. The doses producing hypothermia are similar, if not identical, to those which reverse d-amphetamine responses.

The oral and intravenous dosing of 3 male and 3 female rats each receiving 50 mg/kg (IV) or 250 mg/kg (PO) of ABS201 HCl administered in neutral physiological saline was observed. During the 2 and 24 hour post-dosing clinical observation periods, measurements of core body temperatures were taken.

The following was observed during the period immediately during and following intravenous administration of ABS201: during the dosing period (slow push; >1 min <2 min via the tail vein) the animals receiving ABS201 HCl became noticeably sedate in the body restraining cages; upon removal from the restraining cages, animals were obviously sedated, lacked spontaneous benchtop locomotor activity, assumed a curl position upon handling, and exhibited a greatly impaired, or loss of, the righting reflex; notwithstanding, ptosis was absent; there was no evidence of flaccid paralysis although muscle tone was substantially reduced; pupil reflex was present; the hind limb pinch response was impaired or absent; there was no evidence of parasympathetic responses, e.g., spontaneous urination, defecation, salivation and lacrimation were absent; there was no evidence of acute sympathetic responses, e.g., piloerection; there was no evidence of seizures, either tonic or clonic. The acute effects were short lived with the righting reflex returning by the end of the complete dosing period (approximately 30 min). At the two hour post-dosing observation period, all animals appeared grossly normal although, marked hypothermia was present. At the 24 hour post-dosing observation period, all animals appeared grossly normal; hypothermia was absent. Animals administered ABS201 HCl orally appeared grossly normal at all time points. Thus, the evidence indicates that the acute behavioral appearance and response(s) of animals following the intravenous administration of ABS201 HCl (50 mg/kg) is directly attributable to a rapid and marked central nervous system effect.

### Example 7

### Oral Bioavailability Studies of ABS201

Synthesis of Fmoc-Proline-OH* for Generation of Radioactive ABS201 (FIG. 19). L-Proline (20.7 mg, 0.18 mmol) (Advanced Chemtech) was dissolved in 450 μL of a 10% Na₂CO₃ solution to which 5 mL of EtOH:H₂O (2:98) containing 250 μCi of L-[U-¹⁴C] egrada (Moravek, Brea, Calif.) was added. Fmoc-N-hydroxysuccinimide (Fmoc-Osu) (100 mg, 1.5 eq) in 3 mL dimethoxyethane (DME) was added dropwise to the stirring amino acid solution. The reaction was allowed to stir for 12 hr at room temperature and the DME was removed in vacuo. The remaining aqueous solution was diluted with 10 mL H₂O and extracted with saturated N-butanol (4×10 mL). The butanol extracts were combined and concentrated to give a pale oil. Residual Fmoc-Osu was removed on silica gel eluting with MeOH:CH₂Cl₂ (50:50). Crude Fmoc-Proline-OH* was used without further purification in peptide synthesis.

Study of ABS201 Oral Bioavailability Using Caco-2 Cell Model. Caco-2 cells, derived from a human colorectal carcinoma, spontaneously differentiate into polarized cells that exhibit well-developed microvilli and brush-border enzymes (78). These features make the cells an excellent model of the human small intestine. A strong correlation between uptake in the Caco-2 cell model and oral bioavailability has been identified (79). Studies that focused on the transport of peptides across Caco-2 cells have identified solute-solvent hydrogen bonds as a major determining factor in the permeability of the peptide. The non-natural amino acid technology is designed to reduce solute-solvent interactions, in particular, water solvation that occurs through hydrogen bonding, hence the current modifications should confer enhanced intestinal absorption in Caco-2 cells. The studies described below are designed to evaluate the potential oral bioavailability of the NT(8-13) analogues and the mechanisms of transport responsible for their uptake.

ABS201 is a lead compound for the development of NT(8-13) analogues as novel APDs. ABS201 can therefore function as a prototype for evaluating the cellular uptake of the NT(8-13) analogues. Liquid scintillation counting (LSC) is the preferred method of analysis for these assays, as extraction of the peptide from the cell monolayer is not required and dissolved cell components can be directly analyzed without an extraction protocol that can be inexact, resulting in inconsistent analysis. L-[U-¹⁴C] αegrada was used as the radiolabel for these studies. Proline is easily protected at the α-amine for peptide synthesis with the base-labile Fmoc moiety. In addition, Pro¹⁰ has not been identified as a major site of cleavage of NT(8-13). NT(8-13) analogues that show antipsychotic potential have not included Pro¹⁰ modifications.

To examine the occurrence and mechanism of cellular uptake of the NT(8-13) analogues, Caco-2 cells, a well-established model of the intestinal epithelium can be utilized. These studies were designed to provide insight into the potential for oral activity of the peptide analogues. As described above, the NT(8-13) analogues elicit CNS activity after oral administration. They are the first analogues of NT(8-13) to exhibit oral activity, and these preliminary studies should provide information that aids in the development of future peptide analogues with enhanced oral activity.

The concentration of ABS201 used for these uptake studies, 200 μM, can be chosen for two distinct reasons. The concentration of a 20 mg/kg dose of peptide, delivered in saline (1 mL/kg), is 24 mM. As gavage dosing insures direct administration into the stomach, a concentration only slightly below 24 mM should be seen by the small intestine. Therefore, the concentration added to the Caco-2 cells is well below that theoretically seen in vivo. In addition, the standard circulating blood volume in the rat is 64 mL/kg (82). After gavage dosing, the concentration of a 20 mg/kg dose of peptide circulating throughout the entire rat is 377 μM. For these reasons, 200 μM is determined to be a physiologically relevant concentration to study ABS201 uptake in vitro.

### Example 8

### Preclinical Studies of ABS201

Receptor screening. Three separate concentrations of ABS201 (10⁻⁹, 10⁻⁷ 10⁻⁵ M) were screened individually against the following 16 receptors: adrenergic (alpha1, alpha 2, beta), dopamine, histamine (H1, H2, H3), muscarinic (central, peripheral), nicotinic, opioid (nonselective), orphanin, serotonin (transporter, nonselective), monoamine oxidase (A, B). No displacement of the receptors' endogenous substrate were observed. Hence ABS201 does not appear to bind with any of these receptors. In contrast, the ABS201 has nM affinity for the target receptor (NTR₁).

Liver Receptor Screening (Stanford Research Institute Study B213-06). ABS201 (10⁻³, 10⁻², 0.1 M) was screened against the following liver CYPs: CYP1A2, CYP2B6, CYP2C9, CYP2C19, CYP2D6, CYP3A4. No inhibition of substrate binding at any concentration off ABS201 to these enzymes was observed, indication a lack of potential for drug-drug interactions.

Blood Distribution and Metabolite Identification. ABS201 was added to freshly isolated whole rat blood to a concentration of 100 μg/mL, allowed to partition, and the cellular fraction was removed by centrifugation. ABS201 at this concentration distributes almost evenly between the cellular and serum fractions. No metabolites of ABS201 were been detected, consistent with previous experiments in which a very long serum/plasma half-live was demonstrated.

Maximum Tolerated Dose. ABS201 was administered to rats at IV doses up to 100 mg/kg and oral doses up to 500 mg/kg. No adverse effects of the compound (body weight loss, mortality, abnormal clinical evaluations panel) were seen out to 48 hr post administration. These experiments thus define lower limits for the MTD of ABS201 at 100 times the ED₅₀ for the compound in antipsychosis and other tests reflective of brain activity.

Genetic Toxicology. The micronucleus study, mouse lymphoma study and Ames study were conducted. The micronucleus study evaluated the synthetic NT peptide for its potential to cause genetic damage as manifested by induced micronucleated polychromatic erythrocytes in mouse bone marrow cells. The mouse lymphoma study evaluated the synthetic NT peptide for its potential to cause mutations at the thymidine kinase locus of L5178Y TK+/−mouse lymphoma cells, both unactivated and under S9 metabolic activation conditions. The Ames study evaluated the synthetic NT peptide for its potential to cause mutations in the histidine operon of Salmonella typhimurium strains TA98, TA 100, TA 1535 and TA 1537 and the tryptophan operon of Escherichia coli strain WP2 uvrA, both unactivated and under S9 metabolic activation conditions. Experimental protocols are as follows.

In the micronucleus study, 90 CD-1 mice used in the study consisted of 10/sex/group in Groups 1-4 (control, low-, mid-, and high-dose) and 5/sex in the positive control group. Test peptide as administered orally, the positive control, Cyclophosphamide at 80 mg/kg, as administered orally. Approximately 24 and 48 hours after dosing, mice were killed and bone marrow obtained from both femurs. A slide of a bone marrow suspension was prepared, stained with Wright-Giemsa stain, dried, and scored blind. The number of polychromatic erythrocytes (PCE) among the total erythrocytes (PCE+normochromatic erythrocytes (NCE)) were determined for each animal by counting at least 200 erythrocytes. The number of micronucleated polychromatic erythrocytes (MPCE) then were scored for 2000 PCE per animal. A positive result was obtained, if there is a positive dose-response trend or statistically significant increase in the number of MPCE at one or more dose levels compared to controls.

In the mouse lymphoma study, the standard rat liver S-9 was prepared by inducing male Sprague-Dawley rats with Aroclor-1254 or Phenobarbital and/or β-naphthoflavone. Positive controls included hycanthone methanesulfonate (HYC), which induces mutations at the TK locus without metabolic activation, and 7,12-Dimethylbenz[α]anthracene (DMBA), which induces mutations at the TK locus with metabolic activation. A range finding test was performed to identify the ABS201 concentrations that produce 0-100% toxicity. The positive controls (HYC and DMBA) and five concentrations of ABS201 were used in the assay with and with metabolic activation. Cells were exposed to each ABS dose, and the supernatant obtained, and incubated for 20 and 44 hours. After a 2-day expression period the cultures were cloned with the restrictive agent trifluorothymidine (allows the growth of TK−/− cells only) or vehicle control. The mutation frequency and induced mutation frequency were calculated. A positive response was obtained if at least one culture had a MF that was two times or greater than the average MF of the corresponding solvent control cultures and the response was dose dependent. A confirmatory assay was performed without S-9 activation to confirm Mutation Assay results.

In the Ames study, the synthetic NT peptide analogue was evaluated for its potential to cause mutations in the histidine operon of Salmonella typhimurium strains TA98, TA100, TA1535, TA1537 and the tryptophan operon of Escherichia coli strain WP2 uvrA. In brief, the standard rat liver S-9 was prepared by inducing male Sprague-Dawley rats with Aroclor-1254 or Phenobarbital and/or/3-naphthoflavone. Bacterial strains used were Salmonella typhimurium strains TA98, TA100, TA1535, TA1537 and Escherichia coli strain WP2 uvrA. The positive controls included 2-AA, 2-NF, 9-AA, NaAz, and MMS. A solubility/miscibility assay was conducted to determine the maximum achievable concentration of peptide analogue in the selected solvent (water, DMSO, acetone, or ethanol). A range finding test was performed with and without S-9 activation using tester strains TA100 and WP2 uvrA only to identify ABS201 concentrations that produce 0-100% toxicity. The mutation assay was performed using the four Salmonella typhimurium strains (TA98, TA100, TA1535, TA1537) and the Escherichia coli strain WP2 uvrA strain using the plate incorporation method of treatment. A response was considered positive if either strain TA98 or TA 100 has a dose that produces a mean reversion frequency greater than or equal to two times the mean reversion frequency of the corresponding solvent control plates or if either strain TA 1535, TA 1537 or WP2 uvrA has a dose producing a three-fold or greater increase in the mean reversion frequency compared to the solvent control frequency.

Initial data indicate that ABS201 is inactive in the all three assays.

hERG Assay. The objective of the hERG study is to assess the effects of the test article on the rapidly activating inward rectifying potassium current (I_{Kr}) conducted by hERG (human ether-a-go-go related gene) channels stably expressed in a HEK293 cell line. The method used is as follows. HEK293 cells, stably transfected with hERG cDNA, were used in the study. All experiments were performed at near-physiological temperature (35±2° C.). A positive control (60 nM terfenadine) was applied to two cells (n 2). Cells were be transferred to the recording chamber and superfused with HB-PS solution. The recording chamber and bathing solution were maintained at a temperature of 35±2° C. using a combination of in-line solution pre-heater, chamber floor heater, and feedback temperature controller. Bath temperature were measured using a thermistor probe.

Patch pipettes were made from glass capillary tubing using a P-97 micropipette puller (Sutter Instruments, CA). A commercial patch clamp amplifier was used for whole cell recordings. Before digitization, current records were low-pass filtered at one-fifth of the sampling frequency.

Cells stably expressing hERG were held at −80 mV. Onset and steady state block of hERG current due to ABS201 were measured using a pulse pattern with fixed amplitudes (conditioning prepulse: +20 mV for 1 sec; repolarizing test ramp to −80 mV (−0.5 V/s) repeated at 5 s intervals. Each recording ended with a final application of a supramaximal concentration of the reference substance (E-4031, 500 nM), to assess the contribution of endogenous currents. The remaining unblocked current was subtracted off-line digitally from the data to determine the potency of ABS201 for hERG inhibition.

Initial results indicate that ABS201 is inactive in the hERG study.

Pharmacokinetics and Brain Distribution of ABS201. ABS201 was administered in one IV dose (5 mg/kg) or oral dose (50 mg/kg) to rats. At selected time points, blood was removed or brain harvested and the concentration of the peptide analog determined using LC/MS/MS. It was demonstrated that ABS201 could be detected in the blood and brain up to 120 minutes after both IV and oral administration. The amount in the brain was sufficient to saturate NTR-1 to produce the observed behavioral effects.

ABS201 was cleared from whole blood in two phases with an initial phase and a second phase where compound was measurable at low levels up to 120 minutes following IV administration of 5 mg/kg; the levels of ABS201 in whole blood were below the LLOD at all times following oral administration of the compound (50 mg/kg); the levels of ABS201 in brain were below the LLOD at all times following IV administration; and measurable quantities of ABS201 were detected in brains of 2 of 3 animals 15 minutes post oral administration of 50 mg/kg.

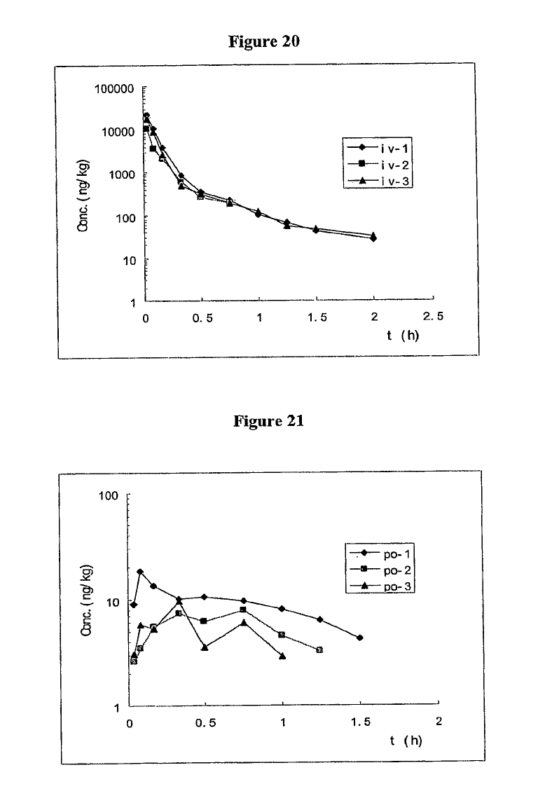

The plasma concentration versus time curve for IV and oral administration of ABS201 are shown in FIGS. 20 and 21. Following IV injection, the mean value of systemic clearance was 2.45 L/hr/kg, which corresponded to 74.02% of rat hepatic blood flow. The mean values of half-life and V_{z} were 0.29 hr and 0.87 L/kg. ABS201 distributes well into the tissues. The volume of distribution at terminal phase was 0.87±0.27 L/kg, which was similar to the total body water (0.67 L/kg) in the rats. The bioavailability of ABS201 following oral administration was 0.07% when in 0.9% saline solution. The mean value of half-life was 0.99 hr.

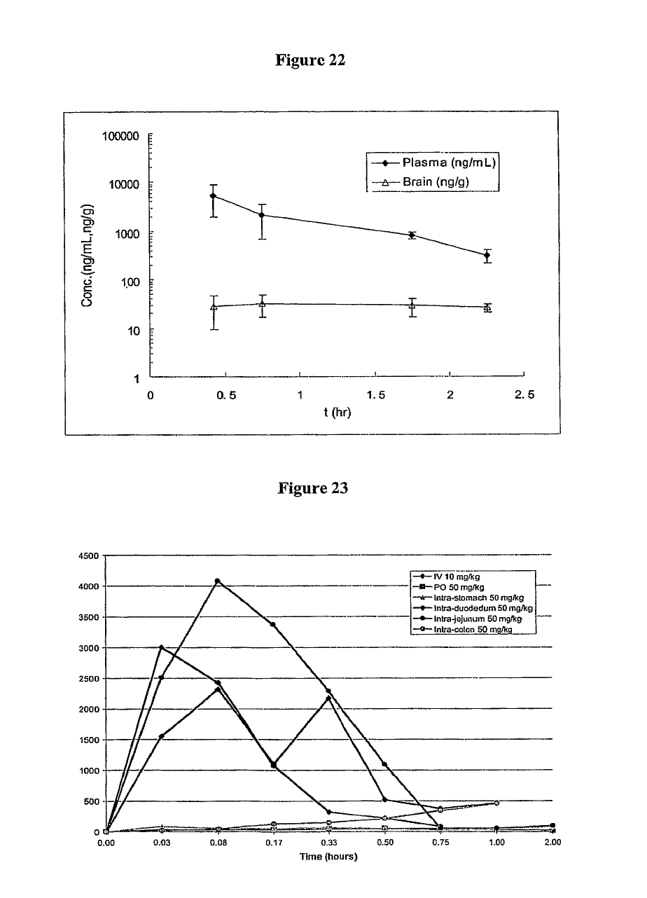

For pharmacokinetics studies of ABS201 in the brain, plasma samples and brain samples were collected from SD rats at four time points following intravenous infusion of test article ABS201-2HCl. These samples were then used for the determination of plasma and brain drug levels by LC/MS/MS for estimating BBB penetration and pharmacokinetic parameters. Time-course results are shown in FIG. 22. Following a 15 min intravenous infusion of ABS201.2HCl at 100 mg/kg, the value of systemic clearance was 22.77 L/hr/kg, which corresponded to 6.88 fold of rat hepatic blood flow (3.31 L/hr/kg). The value of half-life (t½) for ABS201.2HCl was 0.49 hr. The mean value of Cₘₐₓ (at 25 minute) following intravenous infusion at a dose of 100 mg/kg was 5459.78 ng/L. The value of AUC_{0-∞} was 4391.09 hr*μg/L.

ABS201.2HCl distributes well into the tissues. The volume of distribution at terminal phase was 16.10 L/kg, which was greater than the total body water (0.67 L/kg) in the rat.

Following a 15 min intravenous infusion of ABS201.2HCl at 100 mg/kg, the values of Cₘₐₓ (at 45 minute) and AUC₀₋ₜ in brain were 32.93 ng/g and 60.96 hr*ng/g, respectively. The value of half-life (VA) for ABS201:2HCl was 4.85 hr. The brain-to-plasma ratio from 25 min to 135 min following intravenous infusion ranged from 0.0015 to 0.1453. These results demonstrate that ABS201 is selectively taken up into the brain, as it has a significantly longer half-life than in the blood. The brain half-life is in order with the half-lives of the behavioral effect seen for ABS201.

The plasma and brain pharmacokinetics of ABS201 was also studied following IV administration of 1 mg/kg and, oral administration of 30 mg/kg of ABS201 to non fasted rats. The results of this study indicated that: ABS201 was rapidly cleared from plasma following IV administration with a t_{1/2} of about 5 minutes; the levels of the compound decreased below the LLOD by 45 min; the levels of ABS201 were below the LLOD at all times following oral administration; and the levels of ABS201 in brain were below the LLOD at all times following both IV and PO administration.

In vitro metabolism and compartmentalization of ABS201. To evaluate the distribution of ABS201 in blood, the extent of protein binding in plasma, and to gain a preliminary assessment of the metabolism of ABS201 in blood and plasma. The results of this study demonstrated: little or no binding of ABS201 to plasma proteins following a 5 or 30 min incubation at 37° C.; no evidence of metabolism of ABS201 in whole blood or plasma following a 5 or 30 min incubation at 37° C.; and a rapid distribution of about 33 percent of ABS201 into blood cellular elements when whole blood was incubated with ABS201 for 5 or 30 min at 37° C.

Evaluation of the Site of Uptake of ABS201 from PO Administration Brain. With the goal of ultimately increasing the overall oral bioavailability of ABS201 through formulation, the site of uptake in the gut was evaluated. The objective of this study was to collect plasma samples from SD rats at various time points following intra-stomach, intra-duodenum, intra-jejunum, and intra-colon administration of test article ABS201.2HCl in the in situ looped models. These samples were used for the determination of plasma drug by LC/MS/MS for estimating pharmacokinetic parameters and segmental absorption. The bioavailability of intra-stomach, intra-duodenum, intra-jejunum, intra-colon administration relative to intravenous administration and gavage was calculated. As can be seen in FIG. 23, ABS201 is absorbed almost entirely in the intestine, with intra-duodenum exhibiting the maximal amount of uptake. Virtually no ABS201 is taken up in the stomach.

Summary. Substitution of the N-terminal α-azido group of homolysine (Scheme 1) of ABS13 with the methyldesamino derivative 43 (Scheme 2) to produce ABS201 resulted in a molecule possessing important characteristics of a potential antipsychotic. In particular, ABS201 is highly selective for NTR-1 and exhibits effective antipsychotic activity in key rat models of psychosis when administered either IP or orally. ABS201 exhibited a 300% increase in central activity when administered orally, and achieved a more rapid response with oral versus IV injection (these unique attributes are attributable to the desamino modification). ABS201 does not cause catalepsy or induce drug resistance. Initial rat toxicity and mutagenesis experiments have been negative.

The pharmacokinetics, compartmentalization, and possible metabolism of ABS201 were evaluated in vitro and in vivo. The results suggest little or no metabolism, and complex pharmacokinetics which indicate that the compound initially undergoes a rapid clearance from blood, followed by a longer lasting, deep compartment phenomenon.

Also, the pharmacodynamic response of ABS201 is long acting (2-4 hr) following both IV and PO administration; the acute effects of IV ABS201 are mediated via central nervous system; the compound does not appear to be metabolized upon co-incubation with plasma or whole blood; ABS201 partitions between the aqueous and cellular phases of blood in vitro; the PK profile of ABS201 in whole blood is consistent with a two phase clearance process; and the pharmacodynamic response which has been observed is likewise consistent with a two phase clearance process.

ABS201 is preferentially taken up into the brain, presumably through an active transport process, as it is lost from the brain at about 10% the rate that it is lost from the blood. Given the long apparent half life of ABS201 in rat brain (detectable to 6 hours post dosing), and evidence that the drug forms a depot in blood elements (i.e., a slow release delivery system for the drug) the compounds of the invention, such as the semisynthetic peptide ABS201, could be administered on a once or twice daily basis.

### Example 9

### Biological Improvements Resulting from Substitution of —H for —CH₃ in Bioactive Peptides

A series of amino acid analogues of Arg and Lys have been created. See U.S. Pat. Nos. 6,043,218; 6,358,922; 6,566,330; 6,783,946; 6,858,396. When substituted for Arg8 in NT(8-13) a series of compounds have been generated that possess both antipsychotic and analgesic activity. Examples of these compounds can be found in. Table 10 below (ABS202, ABS203, ABS204, ABS206). In addition to the non-natural side chains, it was demonstrated that substitution of a —CH₃ for the N-terminal —NH₂ resulted in the most active compounds for both indications. See PCT/US2005/021580, filed on Jun. 17, 2005, and U.S. Provisional Patent Application Ser. No. 60/581,333, filed on Jun. 17, 2004.

A further set of compounds have been generated in which a terminal —H was substituted for the —CH₃ to evaluate the role of the —CH₃ in the biological activity of these compounds (ABS226, ABS227, ABS228, ABS230, Table 10). The structures of compounds ABS202 and ABS203 are shown in Table 8. Compounds ABS226 and ABS227 differ from ABS202 and ABS203, respectively, in that the N-terminal —CH₃ groups of ABS202 and ABS203 are replaced by —H groups in ABS226 and ABS227. The structure of ABS204 is 30-Arg-Pro-Tyr-Ile-tertLeu-COOH, in which 30 refers to the non-natural residues shown in Scheme 2. The structure of ABS206 is N-Me-Lys-Arg-Pro-Tyr-Ile-tertLeu-COOH. Compounds ABS228 and ABS230 differ from compounds ABS204 and ABS206, respectively, in that the N-terminal CH₃ groups of ABS204 and ABS206 are replaced by —H groups in ABS228 and ABS230.

Each of compound ABS226, ABS227, ABS228 and ABS230 was evaluated against its parent compound ABS202, ABS203, ABS204, ABS206, respectively, for antipsychotic and analgesic activity in appropriate rat models. The former activity is most easily monitored by measuring the extent of induced hypothermia (core body temperature loss), which is a measure of NTR-1 binding and agonism and thus antipsychotic activity. Hypothermia is followed routinely over time through insertion of a thermometer probe hooked up to a digital readout box into the anus of the rats. Analgesia is monitored by the hotplate assay, in which hotplate measurements are taken on a walled-in metal surface that is maintained at 52° C. The latency between the time the rat was placed on the surface and when it licks its back paw is measured. If the animal doesn't lick its hind paw in 30 seconds, it is removed and a maximum positive effect (or MPE) of 100% is recorded. Intermediate responses achieve MPEs between 0-100%, with saline controls defined 0% latency.

TABLE 10

| | | | Hypothermia | Hypothermia | | % MPE |
|---|---|---|---|---|---|---|
| Compound¹ | N-terminus | Position 8² | (Δ ° C.) | (tmax, min) | % MPE | (tmax, min) |
| ABS202 | —CH₃ | hLys-Me | −4.00 | 150 | 100 | 45 |
| ABS226 | —H | hLys-Me | −4.25 | 150 | 40 | 45 |
| ABS203 | —CH₃ | hLys-Me2 | −1.0 | 150 | 61 | 60 |
| ABS227 | —H | hLys-Me2 | −1.7 | 135 | 29 | 60 |
| ABS204 | —CH₃ | hLys-Me3 | −1.6 | 150 | 20 | 45 |
| ABS228 | —H | hLys-Me3 | −2.0 | 120 | 60 | 75 |
| ABS206 | —CH₃ | Lys-Me | −1.8 | 180 | 56 | 60 |
| ABS230 | —H | Lys-Me | −3.8 | 180 | 100 | 30 |

¹All compounds had had identical 9-13 residues of -Arg⁽⁹⁾-Pro⁽¹⁰⁾-Tyr⁽¹¹⁾-tertLle⁽¹²⁾-Leu⁽¹³⁾-COOH
²Indicates a particular patented non-natural analogue of Arg or Lys.

As seen in Table 10, substitution of —H for —CH₃ has an effect in each case. ABS226 has about the same antipsychotic potential as ABS202, but its much smaller analgesic activity is resulting from poorer NTR-2 binding. Thus the compound is more selective for NTR-1 and a better antipsychotic candidate. A similar effect is seen with ABS203 and ABS227. ABS228 has a better analgesic activity than ABS224, hence it is more selective for NTR-2. ABS226 is more active in both potential indications.

To summarize, non-natural Arg and Lys amino acids with —H, when substituted for —CH₃ or —NH₂ in a peptide of biological activity, can produce better pharmaceutical candidates as a result of increased in overall activity of the compounds, or increased selectivity for the receptor subtype of interest.

### Example 10

### Assays for Analgesic Activity of NT Derivatives

Animals. Experimentally naïve male Sprague-Dawley rats from Charles River Laboratories, Raleigh, N.C. (230-260 g) or Harlan, Prattville Ala. (240-280 g), were maintained 4 per cage in animal quarters on a regular light/dark cycle (lights on 0600-1800) with ad libitum food and water for approximately 1 week before the experiment. The range of body weights on the 2 days of testing was 230-260 grams. The experiment was done on one day, with animals allowed to recover for three days before experiments are repeated.

Peptide Preparation. Test articles are prepared at the desired concentration in saline for either IP injection or gavage administration with N=6 for each dose and vehicle. Morphine (5 mg/kg) is used as a control for certain protocols. Specifically, test peptide was suspended in 0.9% NaCl and injected I.P. in a volume of 1 mL/kg (rat weights, 230-260 g) to give a dose of 10 mg/kg. Apomorphine HCl (Sigma-Aldrich, St. Louis, Mo.) was dissolved in distilled water at a concentration of 5 mg/mL within 40 minutes of administration for injection at 1 mL/kg S.C. to give a dose of 5 mg/kg. When complete ED₅₀s are determined, dosing range of the peptide will be 10, 3, 1, 0.3, 0.1 of the estimated ED₅₀, covering two log orders of range.

The Hotplate Assay. The hotplate assay, a standard rat model for chronic pain (see Le Bars et al., Pharmacol. Rev. 53: 597-652 (2001) and Chapman et al., Pain 22:1-31 (2002)), was performed as follows. A hotplate maintained at 52° C. to 53° C. was enclosed on four sides in a plexiglass chamber that extended 6 in. above the plate. Animals were placed on the plate, and the latency between the time a rat was placed on the surface and when it lifts or licks a back paw (indicative of pain sensation) was determined. Animals are removed immediately at the cutoff latency of 30 seconds. Assays are scored as the percent of maximal possible effect (% MPE) defined as 100%, which corresponds to the rats remaining on the hotplate for 30 seconds without responding. Thus, % MPE are calculated using the following equation: % MPE=[(post-drug latency−pre-drug latency)/(cut-off pre-drug latency)]×100%. N=6 or greater for all experiments.

Values for each set of experiments will be submitted to analysis of variance, with dose (vehicle and 5 levels) as the factor. Following significant effects in analysis of variance, each dose will be compared to vehicle by the Newman-Keuls test. An effect will be considered significant if p<0.05.

The Tail Flick Assay. Each of 6 rats per experiment were placed in a Plexiglas restraint box. A standard laboratory hot water bath was maintained at 49° C. The distal 3 cm of the rat's tail was submerged into the water, and the time from insertion to tail flick or removal of tail from the water was recorded. Animals are removed immediate4ly at the cutoff latency of 10 seconds. MPEs were calculated, with a MPE of 100% corresponding to the rat's tail remaining in the water bath for 10 seconds. This was repeated 3 times. The tail was dried and allowed to rest for 20 seconds between trials. The rat was then given the appropriate amount of peptide and the experiment repeated at 30 and 60 minutes. Treatment conditions were distributed over the study in a counterbalanced design to control for such factors as calendar period and order of dosing. N=6 or greater for all experiments.

Formalin Assay. Six naïve rats were assigned to a single treatment condition, given either a test peptide as detailed above, or saline (control) and placed into Plexiglas observation cages (43 cm×24 cm×20 cm) without bedding material and their behavior monitored for 15 min. Animals were scored every minute on the following scale: 0=forepaw flat on floor, 1=forepaw just touching floor, 2=forepaw held up off of floor, and 3=forepaw licked or bitten. Following this baseline determination, animals were injected with a 5% solution of 37% formaldehyde (50 mL of 5% formaldehyde in sterile saline prepared in 1.0 mL syringes with 26G3/8 needles) subcutaneously into the right forepaw. The animals were immediately be placed into the observation cages, and their behavior recorded over the following 30 minutes. After each experiment, rats were euthanized with CO₂ followed by cervical dislocation. An average Pain Rating over time versus the baseline and formalin-only controls for each peptide tested were plotted, and statistical analysis were performed using a one-way analysis of variance (ANOVA). Differences were considered statistically significant at the 5% confidence interval. N=6 or greater for all experiments. A biphasic behavioral response has been established, with an initial phase (approximately 3 minutes after injection) reflecting the initial pain stimulus followed by a secondary phase after about 30 minutes reflecting sensitization due to instigation of inflammatory processes.

Chung assay. To assess tactile thresholds, rats are placed in a clear plastic, wire mesh-bottomed cage, divided into individual compartments. Animals are allowed to acclimate and then baseline thresholds are assessed prior to drug treatment. To assess the 50% mechanical threshold for paw withdrawal, von Frey hairs are applied to the plantar mid-hind paw, avoiding the tori (footpads). The eight von Frey hairs used are designated by [log(10*force required to bend hair, mg)] and range from 0.4-15.1 grams (#'s 3.61-5.18). Each hair is pressed perpendicularly against the paw with sufficient force to cause slight bending, and held for approximately 6-8 seconds. A positive response is noted if the paw is sharply withdrawn. Flinching immediately upon removal of the hair is also considered a positive response. Absence of a response (“−”) is cause to present the next consecutive stronger stimulus; a positive response (“+”) is cause to present the next weaker stimulus. Stimuli are presented successively until either six data points are collected, or the maximum or minimum stimulus is reached. If a minimum stimulus is reached and positive responses still occurred, the threshold sis assigned an arbitrary minimum value of 0.25 grams; if a maximum stimulus is presented and no response occurred, a maximum threshold value of 15 grams is assigned. If a change in response occurs, either “−” to “+” or “+” to “−”, causing a change in the direction of stimulus presentation from descending to ascending or vice-versa, four additional data points are collected subsequent to the change. The resulting pattern of responses are tabulated and the 50% response threshold computed using the formula: log(threshold, mg×10)=Xf+kd; where:
Xf=value of the last von Frey hair applied;
k=correction factor based on pattern of responses (from calibration table)
d=mean distance in log units between stimuli.
   Based on observations on normal, un-operated rats and sham-operated rats, the cutoff of a 15.1-g hair is selected as the upper limit for testing.

### Example 11

### ABS201 as a Potential Analgesic

The antipsychotic and hypothermic effects of NT and derivatives are mediated through NTR-1 agonism while analgesic effects are mediated through NTR-2 agonism. To be a viable analgesic, a NT derivative should in vitro bind with high affinity to NTR-2 and in vivo be orally available and stable. ABS201 fulfills all of these requirements—having sub-micromolar NTR-2 affinity prolonged plasma stability and orally bioavailability as shown in Table 11.

TABLE 11

| Indication | NT[8-13] | ABS201 |
|---|---|---|
| NTR-1 receptor binding | 0.21 ± 0.01 | 3.92 ± 0.01 |
| (ED₅₀, nM)¹ | | |
| Functional | Yes | Yes |
| NTR-1 Agonist² | | |
| NTR-2 receptor binding | 4.1 ± 0.01 | N.D. |
| (ED₅₀, nM)¹ | | |
| Functional | Yes | Yes |
| NTR-2 Agonist² | | |
| Plasma stability | 5 | >10,000 |
| T_{1/2} (min)³ | | |
| Inhibits locomotor | CNS administration | CNS, IP, Oral |
| activity⁴ | | Administration |
| Hyperlocomotion | CNS administration | CNS, IP, Oral |
| Inhibition⁴ | | Administration |
| Catalepsy⁴ | No | No |
| Toxicity⁵ | — | No |
| | | (>100 x ED₅₀) |
| Dose Tolerance⁶ | — | No |

¹Competitive NTR binding affinities versus radiolabeled NT[8-13][103]
²Induction of cAMP and inositol phosphate production (Orwig, Ph.D. dissertation, 2005).
³Quantitative MALDI-MS assay developed in our laboratory [53].
⁴Behavioral assay methods as described in [103].
⁵Single acute dose at 100x ED₅₀ administered I.P. [unpublished]
⁶10x ED₅₀ administered daily over 5 days. [103].

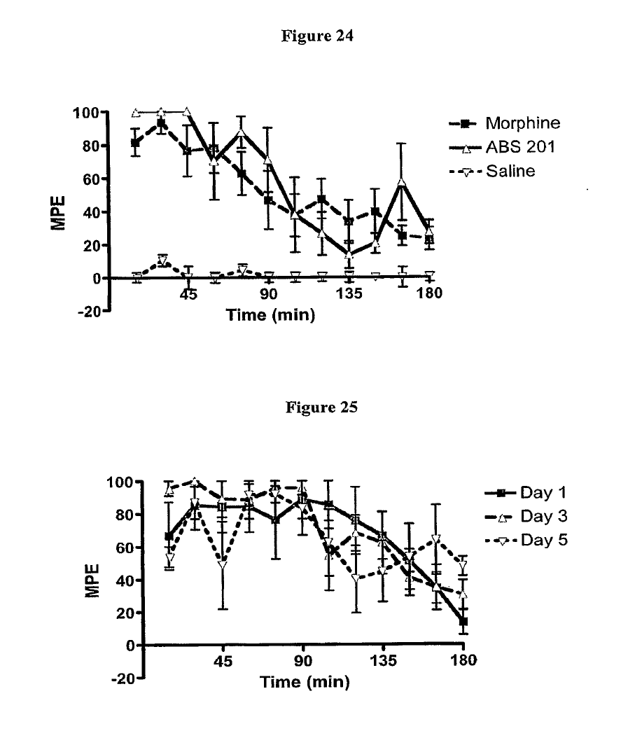

ABS201 also exhibits analgesic activity as indicated by % MPE in the hotplate assay comparable in extent and duration to morphine (FIG. 24). Of interest, however, are differences in the animals' affect after administration of these peptides. Rats given morphine looked “drugged”, becoming more rigid and completely unresponsive to environmental stimuli, while animals give ABS201 appeared simply to be relaxed. The rats do not become tolerant to the analgesic activity of ABS201 as rats dosed for five days straight show statistically identical % MPEs as when initially dosed (FIG. 25). This is especially noteworthy as the rats are not experimentally naïve by the end of the dose time course, which can result in them learning to avoid the hotplate and mask the potential for analgesia. Finally, the analgesic activity of ABS201 is not linked to its hypothermic effect since the two do not show parallel kinetics—specifically the analgesic effect wears off while the hypothermic effect is still strong. This clearly indicates that the effects are mediated by different receptors. Overall, these data indicate the usefulness of ABS201 as a potential analgesic and antipsychotic.

### Example 12

### Analgesic Peptides Having Side Chain Guanidinium Groups

By virtue of the fact that ABS201 binds both NTR-1 and -2 with high affinity, there is a chance that effects associated with NTR-1 binding may make the peptide less desirable as an analgesic. These effects include induction of systemic hypothermia in rats, which is actually a very small effect in humans (D. Feifel, personal communication), and antipsychotic potency, which may not be undesirable. A peptide highly selective for NTR-2 that maintains all of the favorable attributes of ABS201 would be a “cleaner” analgesic candidate.

In the process of developing an NTR-2 selective ligand, the peptides shown in FIG. 26 were evaluated. These peptides exhibited enhanced NTR-2 to NTR-1 binding affinities, and the relative selectivity for the two receptors are provided in Table 12.

TABLE 12

| | | | NTR-1/NTR-2 |
|---|---|---|---|
| Peptide | NTR-1 (nM) | NTR-2 (nM) | selectivity |
| NT[8-13] | 0.21 ± 0.01 | 4.1 ± 0.01 | 19.5 |
| ABS1 | 0.38 ± 0.07 | 0.7 ± 0.02 | 1.8 |
| ABS13 | 5.7 ± 0.37 | 11.1 ± 0.1 | 1.9 |
| ABS201 | 11.2 ± 0.3 | n/d | n/d |
| ABS15 | 0.4 ± 0.1 | 0.9 ± 0.2 | 2.5 |
| ABS16 | 0.3 ± 0.05 | 0.67 ± 0.07 | 2.2 |
| ABS17 | 0.25 ± 0.07 | 0.3 ± 0.1 | 1.2 |
| ABS19 | 1.2 ± 0.7 | 0.23 ± 0.05 | 0.19 |

¹Competitive

Since NT[8-13] has a 20-fold preference for NTR-1 binding over NTR-2 (Table 12), it is less useful as an NTR-2 selective ligand. However, the data for peptides ABS1, ABS13, and ABS201 indicate that there is a structure-binding effect in varying the α-amino group. Changing the polar —NH₂ (which is protonated at physiological pH significantly adding to its hydrophilicity) of ABS1 to nonpolar, uncharged —N₃ (ABS13) resulted in about a 10-fold improvement of NTR-2 to NTR-1 selectivity. In addition, as the arginine side chains get larger and more nonpolar, significantly better binding to NTR-2 resulted. The differences in the arginine side-chain binding site of NTR-2 is illustrated in the progression of ABS15, ABS16, ABS17, ABS19. ABS19 has a 5-fold selectivity for NTR-2, and an overall selectivity improvement of about 100-fold over NT[8-13]. In addition, significant loss of receptor binding affinity is tolerated in this system as ABS201, which binds 50-fold less effectively than it's parent, still exhibited saturable effects in the rat schizophrenic models when given orally. ABS201 is about three times as physiologically active as ABS13 in the rat behavioral models of schizophrenia when administered orally, clearly defining this as the best substitution for the α-amino group. Based on this rationale, peptides designed to be better NTR-2 selective ligands are created and their structures are presented in FIG. 27. All of these peptides contain R=Arg-Pro-Tyr-tertleu-Leu-COOH and the N-terminal methyl group of ABS201, which are required for the potential of oral activity. These peptides also incorporate added steric bulk around the guanidinium group, elements that demonstratively improve NTR-2 versus NTR-1 selectivity versus NT[8-13] (as does the N-terminal methyl group). Desirable analgesic peptides are those which (1) exhibit analgesic activity comparable to or better than ABS201 in the hotplate, tail flick and formalin assays when IP injected, (2) exhibit activity when orally administered, (3) have desirable NTR-1 and NTR-2 binding activities, and (4) exhibit plasma stability.

### Example 13

### Analgesic NT Peptide Analogues

The process of identification of ABS201 enabled definition of the key structural parameters necessary for biological stability and blood brain barrier crossing. Accordingly, a group of neurotensin (NT)[8-13] derivatives that incorporate structural elements that may enhance IP or oral bioavailability were created. Within these parameters, other proprietary structural changes were incorporated that can affect various pharmacological and behavioral parameters relevant to the potential development as therapeutic agents. In sum, peptides with potential for (1) enhanced binding affinities for their receptors, e.g. enhanced binding and selectivity (e.g. NT-derivatives with selectivity for brain NTR-2 have analgesic activity, while those with selectivity for NTR-1 have antipsychotic activity); (2) enhanced biological barrier crossings; (3) enhanced stability; and (4) feasible synthetic costs were identified.

Specifically, chemical changes to the established NT[8-13] derivative included incorporation of an non-natural amino acid residue at position 8. The new peptides were designed to retain the structural elements of the antipsychotic lead ABS201 that are necessary for oral activity while varying the Arg(8) residue and moiety at the N-terminus. Peptides with enhanced analgesic activity, i.e. predicted to have NTR-2 selectivity, were screened via IP injection in the rat hotplate model of analgesia as described above. Initial screening experiments of five peptides using the three different assays for analgesic activity described above indicated that the assays yielded comparable relative activity, with the hotplate assay being the most sensitive. The IP dose chosen was designed to produce a maximal response from active compounds as measured by the % MPE (Maximal Positive Effect). An MPE of 100% indicates that the rat did not feel pain for the extent of time on the hotplate, using a cutoff of 30 seconds. Tmax indicates the time after dosing in which a maximum response was observed. The tmax for all compounds were recorded as this gave an indication of the duration of each compound's analgesic effect. Compounds that exhibited a % MPE of 100% were evaluated with oral dosing. Table 13 provides the sequences of the peptides tested, selectivity for the NTR-1 or NTR-2 as indicated by ability to induce hypothermia, and the results of the Hotplate assay. Compounds with an asterisk exhibited significant activity in the hotplate assay.

TABLE 13
Analgesic Activity of ABS201 Derived Compounds¹

| | | | | | Hypo | | | | |
|---|---|---|---|---|---|---|---|---|---|
| Peptide* | N-terminus³ | Peptide Sequence² | Hypo (I.P.) | Tmax | (oral) | MPE (I.P.)⁴ | Tmax (I.P.)⁴ | MPE (oral)⁵ | Tmax (Oral)⁵ |
| ABS201* | CH₃ | hLys-Arg-Pro-Tyr-t-Leu-Leu | −5.20 | 150.00 | −2.58 | 100 | 15-45 | 39 | 30 |
| ABS202* | CH₃ | 1-Arg-Pro-Tyr-t-Leu-Leu | −4.00 | 150.00 | −2.15 | 100 | 30-75 | 52 | 45 |
| ABS203 | CH₃ | 2-Arg-Pro-Tyr-t-Leu-Leu | −1.00 | 150.00 | ND | 60 | 60 | ND | ND |
| ABS204 | CH₃ | 3-Arg-Pro-Tyr-t-Leu-Leu | −1.60 | 150.00 | ND | 19 | 45 | ND | ND |
| ABS205* | CH₃ | Lys-Arg-Pro-Tyr-t-Leu-Leu | −4.00 | 195.00 | −2.50 | 86 | 15-30 | 66 | 30 |
| ABS206 | CH₃ | 4-Arg-Pro-Tyr-t-Leu-Leu | −1.80 | 180.00 | ND | 55 | 60 | ND | ND |
| ABS207* | CH₃ | 5-Arg-Pro-Tyr-t-Leu-Leu | −2.90 | 165.00 | −1.50 | 84 | 45 | 36 | 60 |
| ABS208* | CH₃ | 6-Arg-Pro-Tyr-t-Leu-Leu | −2.80 | 135.00 | −0.20 | 67 | 135 | 26 | 105 |
| ABS209 | CH₃ | Orn-Arg-Pro-Tyr-t-Leu-Leu | −2.55 | 195.00 | ND | 89 | 60 | IP | IP |
| ABS210* | CH₃ | 7-Arg-Pro-Tyr-t-Leu-Leu | −1.18 | 180.00 | −2.13 | 82 | 60 | 49 | 75 |
| ABS211* | CH₃ | 8-Arg-Pro-Tyr-t-Leu-Leu | −4.20 | 210.00 | −1.15 | 100 | 15, 45-75 | 16 | 120 |
| ABS212* | CH₃ | 9-Arg-Pro-Tyr-t-Leu-Leu | −4.20 | 240.00 | −2.45 | 100 | 30-135 | 39 | 120 |
| ABS213 | CH₃ | Arg-Arg-Pro-Tyr-t-Leu-Leu | −2.65 | 180.00 | ND | 48 | 30 | ND | ND |
| ABS214 | CH₃ | 10-Arg-Pro-Tyr-t-Leu-Leu | −4.20 | 195.00 | −2.40 | 100 | 30-75 | 30 | 15 |
| ABS215 | CH₃ | 11-Arg-Pro-Tyr-t-Leu-Leu | −2.45 | 165.00 | ND | 63 | 45 | ND | ND |
| ABS216 | CH₃ | 12-Arg-Pro-Tyr-t-Leu-Leu | −2.83 | 195.00 | ND | 74 | 120 | ND | ND |
| ABS217 | CH₃ | 13-Arg-Pro-Tyr-t-Leu-Leu | −1.80 | 90.00 | ND | 8 | 45 | ND | ND |
| ABS218 | CH₃ | 18-Arg-Pro-Tyr-t-Leu-Leu | −1.88 | 180.00 | ND | 79 | 45 | ND | ND |
| ABS220 | CH₃ | 15-Arg-Pro-Tyr-t-Leu-Leu | −4.30 | 180.00 | −1.30 | 55 | 45 | 36 | 15 |
| ABS221 | CH₃ | 16-Arg-Pro-Tyr-t-Leu-Leu | −1.80 | 165.00 | ND | 76 | 60 | ND | ND |
| ABS224 | CH₃ | alkene-Arg-Pro-Tyr-t-Leu-Leu | −1.30 | 195.00 | ND | 45 | 150 | ND | ND |
| ABS501 | CH₃ | 15-Arg-Pro-Tyr-Ile-Leu | −4.20 | 100.00 | ND | ND | ND | ND | ND |
| ABS700 | | Boc-Arg-Pro-Tyr-t-Leu-Leu | −2.05 | 90.00 | ND | 51 | 90 | ND | ND |
| ABS225* | H | hLys-Arg-Pro-Tyr-t-Leu-Leu | −3.00 | 180.00 | −1.20 | 100 | 15-45 | 38 | 30 |
| ABS226 | H | 1-Arg-Pro-Tyr-t-Leu-Leu | −3.55 | 150.00 | −0.48 | 59 | 45 | 32 | 15 |
| ABS227 | H | 2-Arg-Pro-Tyr-t-Leu-Leu | −1.70 | 135.00 | ND | 29 | 60 | ND | ND |
| ABS228 | H | 3-Arg-Pro-Tyr-t-Leu-Leu | −1.98 | 120.00 | ND | 55 | 75 | ND | ND |
| ABS229 | H | Lys-Arg-Pro-Tyr-t-Leu-Leu | −1.85 | 105.00 | ND | 48 | 60 | ND | ND |
| ABS230* | H | 4-Arg-Pro-Tyr-t-Leu-Leu | −3.75 | 180.00 | −1.10 | 100 | 30 | 36 | 30 |
| ABS231 | H | 5-Arg-Pro-Tyr-t-Leu-Leu | −2.38 | 135.00 | −1.63 | 100 | 30-60 | 20 | 30 |
| ABS232* | H | 6-Arg-Pro-Tyr-t-Leu-Leu | −3.87 | 240.00 | −0.38 | 84 | 45 | 49 | 60 |
| ABS233 | H | Orn-Arg-Pro-Tyr-t-Leu-Leu | −1.40 | 180.00 | ND | 51 | 45 | ND | ND |
| ABS234* | H | 7-Arg-Pro-Tyr-t-Leu-Leu | −4.18 | 240.00 | −0.83 | 100 | 15-120 | 39 | 75 |
| ABS235 | H | 8-Arg-Pro-Tyr-t-Leu-Leu | −2.58 | 165.00 | −2.18 | 100 | 30-75 | 20 | 60 |
| ABS236 | H | 9-Arg-Pro-Tyr-t-Leu-Leu | −2.37 | 180.00 | ND | 100 | 30-60 | IP | IP |
| ABS237 | H | Arg-Arg-Pro-Tyr-t-Leu-Leu | −1.85 | 165.00 | ND | 50 | 30 | ND | ND |
| ABS238 | H | 10-Arg-Pro-Tyr-t-Leu-Leu | −3.80 | 165.00 | −2.08 | 100 | 30, 60-75 | 37 | 30 |
| ABS239 | H | 11-Arg-Pro-Tyr-t-Leu-Leu | −3.53 | 165.00 | −0.78 | 92 | 15 | 32 | 60 |
| ABS240 | H | 12-Arg-Pro-Tyr-t-Leu-Leu | −2.55 | 165.00 | ND | 37 | 45 | ND | ND |
| ABS241 | H | 13-Arg-Pro-Tyr-t-Leu-Leu | −3.35 | 135.00 | −0.75 | 95 | 60 | IP | IP |
| ABS242 | H | 18-Arg-Pro-Tyr-t-Leu-Leu | −2.65 | 135.00 | ND | 76 | 30 | ND | ND |
| ABS243 | H | 19-Arg-Pro-Tyr-t-Leu-Leu | −2.38 | 135.00 | ND | 79 | 60 | ND | ND |
| ABS244 | H | 15-Arg-Pro-Tyr-t-Leu-Leu | −4.25 | 165.00 | −1.50 | 82 | 45 | 26 | 30 |
| ABS245 | H | 16-Arg-Pro-Tyr-t-Leu-Leu | −3.13 | 165.00 | −1.27 | 50 | 45 | ND | ND |
| ABS246 | H | 17-Arg-Pro-Tyr-t-Leu-Leu | −1.68 | 165.00 | ND | 80 | 45 | ND | ND |

¹Hot plate measurements were takend on a walled-in metal surface maintained at 52 C. The latency between the time the rat was placed on the surface and when it licks its back paw is measured. Animals are removed immediately at the cutoff latency of 30 sec, which corresponds to a % MPE of 100%.
²Side chain residue at 8-position as defined in Scheme 3.
³Group substituted for the α-amino group.
⁴% MPE and Tmax of % MPE for compound administered IP at 10 mg/kg.
⁵% MPE and Tmax of % MPE for compound administered orally at 20 mg/kg.
ND = not determined,
IP = in progress

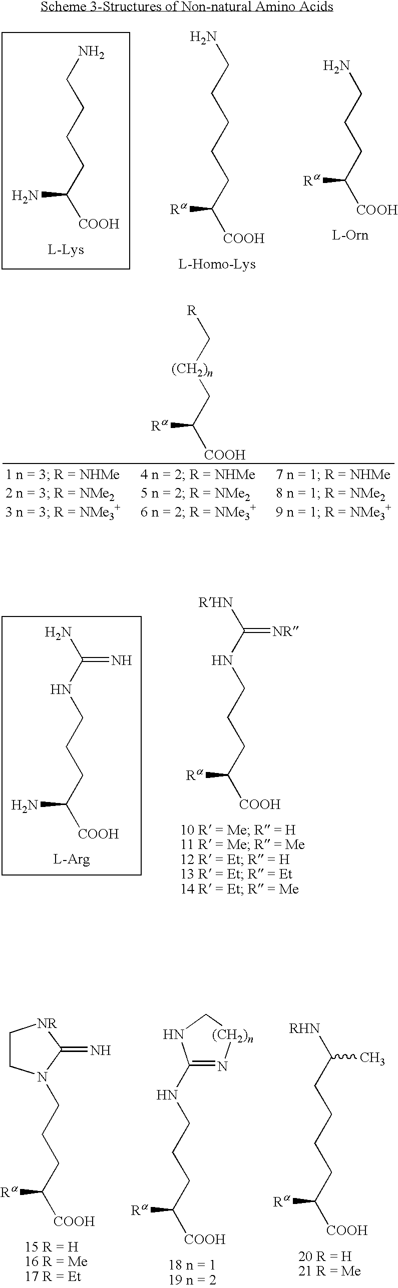

Initial screens of all peptides included (1) evaluating their analgesic ability when dosed IP or orally (indicative of NTR-2 agonism) and (2) evaluating their ability to induce hypothermia when IP, a secondary effect of NTR-1 agonist activity. Peptides that exhibited significant analgesic activity, that is, greater than a set threshold level, when IP dosed were evaluated with oral dosing. The peptides that have high analgesic and low hypothermia-inducing activity when dosed orally, indicating high selectivity for NTR-2, were preferred. However, peptides with high hypothermia activity may show significantly greater oral bioavailability and were also preferred as they were better analgesic candidates, particularly if side effects of NTR-1 binding were minor. A third parameter was that IP analgesics may have different utilities, and potencies, than orally administered peptides. Defined dose-response curves were also considered in selecting an analgesic candidate. Accordingly, the different parameters were weighed, and peptides that were not as “good” at others under the above analysis while showing overall similar profiles were not selected. A secondary screening that involved evaluating analgesia when dosed orally was performed, and the best peptides were subjected to a full dose-response analysis.

Based on the above criteria, peptides ABS201, ABS202, ABS205, ABS207, ABS208, ABS210, ABS211, ABS212, ABS220, ABS225, ABS230, ABS232, ABS234 and ABS239 were identified as preferred analgesic peptides. The structures for these peptides are summarized in the following table.

TABLE 14

| Peptide | N-terminus (Rα) | C-terminus* |
|---|---|---|
| ABS201 | CH₃— | hLys-Arg-Pro-Tyr-tLeu-Leu-COOH |
| ABS202 | CH₃— | 1-Arg-Pro-Tyr-tLeu-Leu-COOH |
| ABS205 | CH₃— | Lys-Arg-Pro-Tyr-tLeu-Leu-COOH |
| ABS207 | CH₃— | 5-Arg-Pro-Tyr-tLeu-Leu-COOH |
| ABS208 | CH₃— | 6-Arg-Pro-Tyr-tLeu-Leu-COOH |
| ABS210 | CH₃— | 7-Arg-Pro-Tyr-tLeu-Leu-COOH |
| ABS211 | CH₃— | 8-Arg-Pro-Tyr-tLeu-Leu-COOH |
| ABS212 | CH₃— | 9-Arg-Pro-Tyr-tLeu-Leu-COOH |
| ABS220 | CH₃— | 15-Arg-Pro-Tyr-tLeu-Leu-COOH |
| ABS225 | H— | hLys-Arg-Pro-Tyr-tLeu-Leu-COOH |
| ABS230 | H— | 4-Arg-Pro-Tyr-tLeu-Leu-COOH |
| ABS232 | H— | 6-Arg-Pro-Tyr-tLeu-Leu-COOH |
| ABS234 | H— | 7-Arg-Pro-Tyr-tLeu-Leu-COOH |
| ABS239 | H— | 11-Arg-Pro-Tyr-tLeu-Leu-COOH |

*See Scheme 3 above for the structure of the N-terminal moiety.

### Example 14

### Evaluation of the Analgesic Effects of Selected NT-Analogues

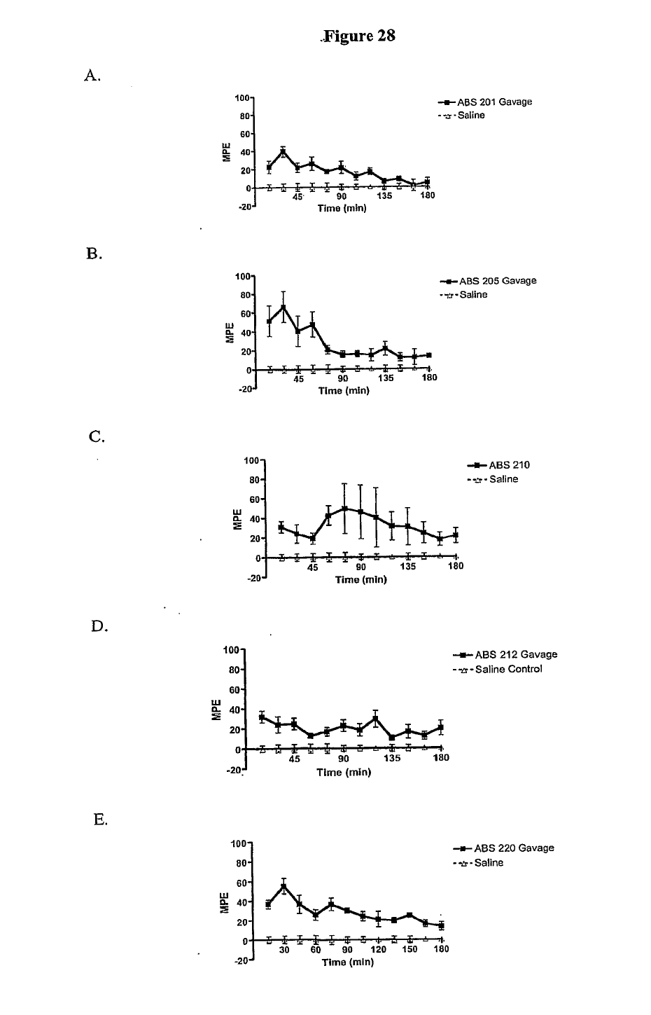

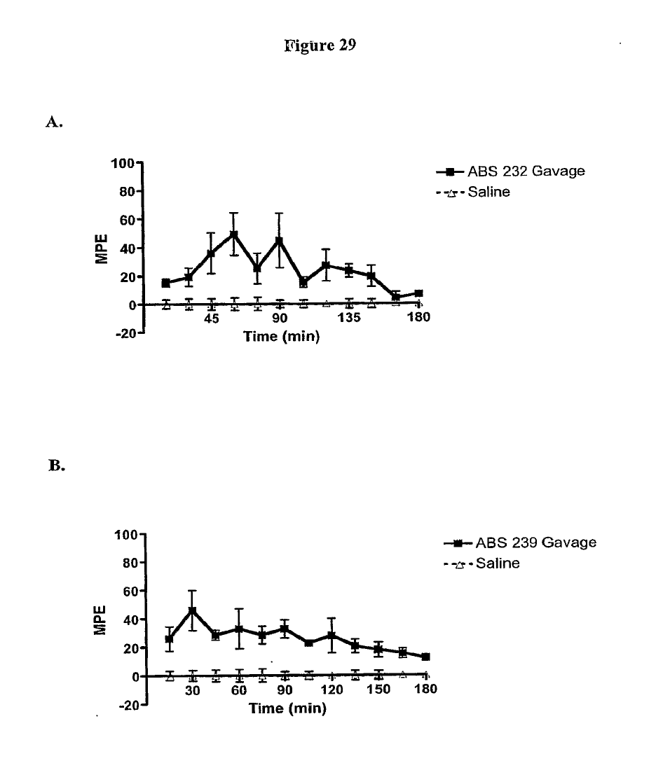

The analgesic effects of ABS201, ABS205, ABS210, ABS212, and ABS220 administered by gavage at a dose of 20 mg/kg are shown in FIG. 28. These results indicate that ABS201, ABS205, ABS210 and ABS220 were as much or more active than ABS212 when given orally. The analgesic effects of ABS232 and ABS239 as determined using the hotplate assay are illustrated in FIGS. 29 A and B.

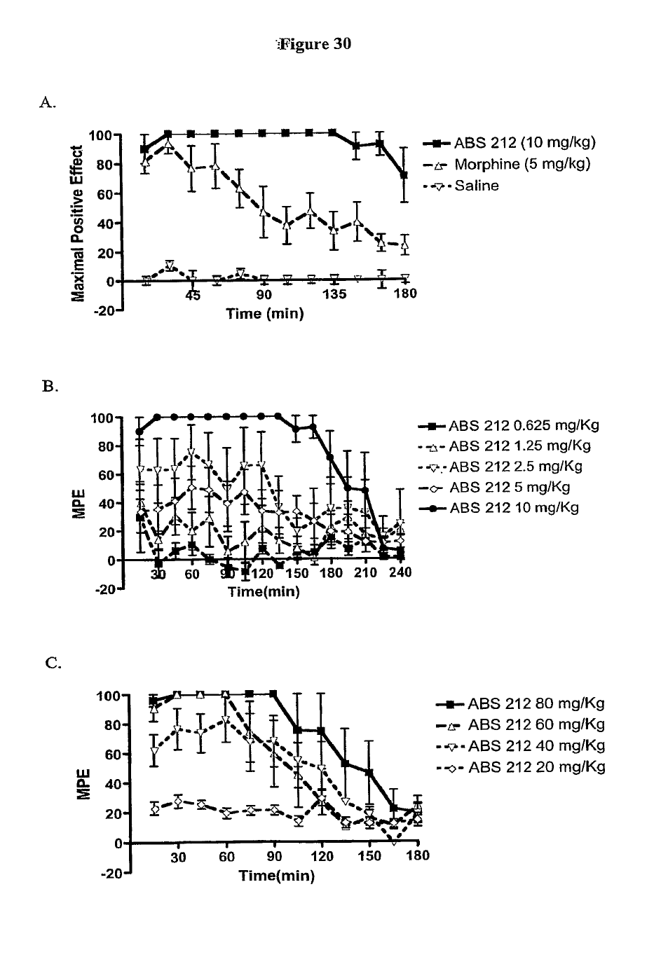

When administed by I.P. injection, however, the most active peptide was ABS212. The analgesic property of ABS212 was compared with that of morphine using the hotplate, tail flick and formalin assays. Results obtained from the hotplate assay and the formalin assays are shown in FIGS. 30 A and B, respectively, while results obtained from the tail flick assay is summarized in Table 15 below.

ABS212 at an approximately equal molar ratio exhibits analgesic activity (% MPE) in the hotplate assay comparable or better in extent and duration to morphine (FIG. 30A). Of interest, however, are differences in the animals' affect after administration of these peptides. Rats given morphine looked “drugged,” becoming more rigid and completely unresponsive to environmental stimuli, while animals give ABS212 appeared simply to be relaxed. In addition, the rats do not become tolerant to the analgesic activity of ABS212 as rats dosed for five days straight show statistically identical % MPEs as when initially dosed (data not shown). This is especially noteworthy as the rats are not experimentally naïve by the end of the dose time course, which can result in them learning to avoid the hotplate and mask the potential for analgesia. FIGS. 30B and C show the dose response data for ABS212 when administered IP or orally, respectively. Approximate ED₅₀s for IP and oral doses are 2.5-5 mg/kg and 30-40 mg/kg respectively.

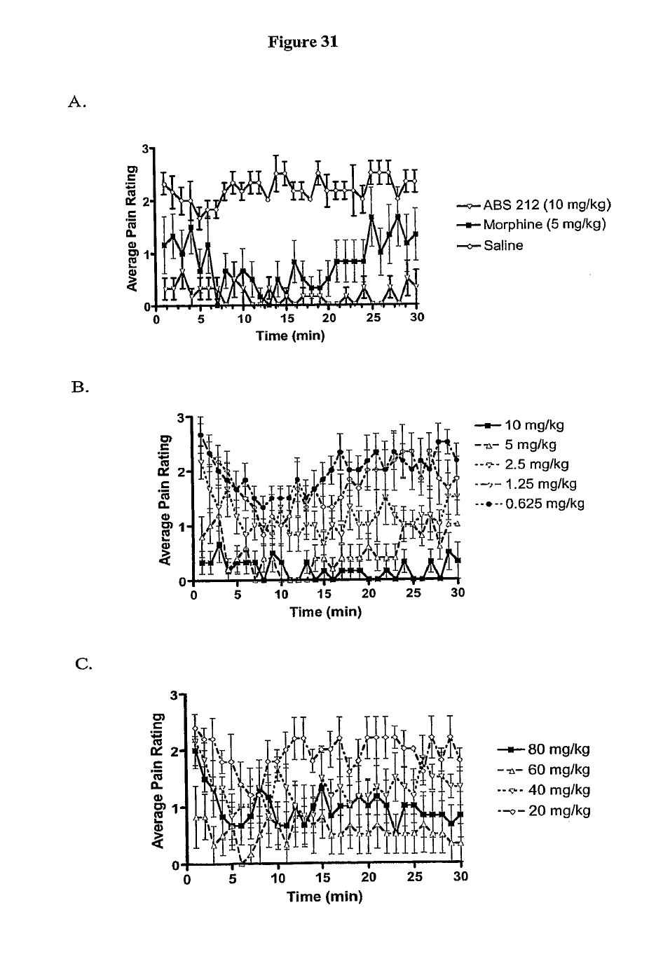

In the formalin assay, ABS212 provides a full analgesic effect for the time course of the experiment (FIG. 31A). As also seen in the hotplate assay, morphine at approximately equimolar concentrations, loses activity after 30 minutes. ABS212, however, has a more pronounced analgesic effect when administered by I.P. injection. FIGS. 31B and C show the dose responses determined using the formalin assay for I.P. and oral administration, respectively. Approximate IP and oral ED₅₀s are in the same range as the assays performed above. The analgesic activity ABS212 appears to be highly selective for NTR-2 as very little hypothermia (a secondary effect of NTR-1 binding) is elicited from this peptide at therapeutic analgesic doses. (In vitro receptor binding studiers are in progress).

In the tail flick assay, ABS212 exhibits statistically significant analgesia at 30 minutes, with an apparent greater effect at 60 minutes, when compared to saline (see Table 15). ABS212 statistically compares to morphine.

TABLE 15
Comparison of ABS212 (10 mg/kg) to Morphine (5 mg/kg) and
Saline in the Tail Flick Assay

| Treatment | MPE (0 min) | MPE (30 min) | MPE (60 min) |
|---|---|---|---|
| Saline | −0.37 +/− 0.57 | 9.7 +/− 5.6 | 14.6 +/− 7.1 |
| Morphine | 0.03 +/− 0.05 | 63.7 +/− 41.9 | 67.5 +/− 25.8 |
| ABS212 | 1.16 +/− 0.05 | 75.4 +/− 28.4 | 88.7 +/− 19.6 |

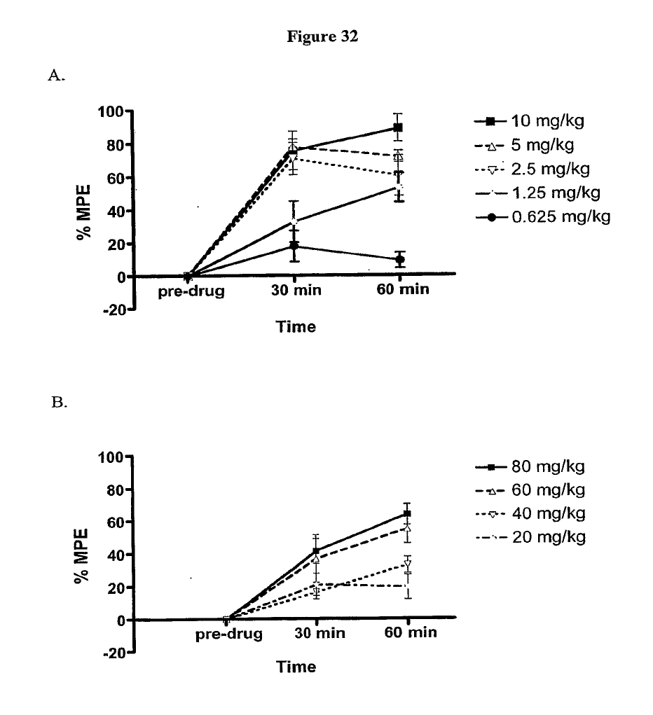

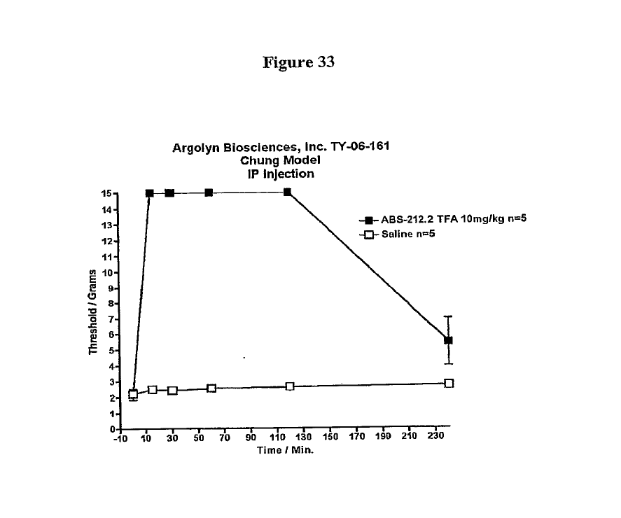

FIGS. 32A and B show the dose response data for ABS212 when administered I.P. and orall, respectively, in the tail flick assay. Approximate IP and oral ED₅₀s are in the same range as seen in the above assay. FIG. 33 demonstrate that ABS212 is highly active in the Chung model for neuropathic pain when administered by I.P. injection. In sum, ABS212 is a highly effective analgesic in the four key rat models for the spectrum of pain states: acute (hotplate, tail flick), chronic (formalin) and neuropathic (Chung).

### Example 15

### Preclinical Characterization of ABS212

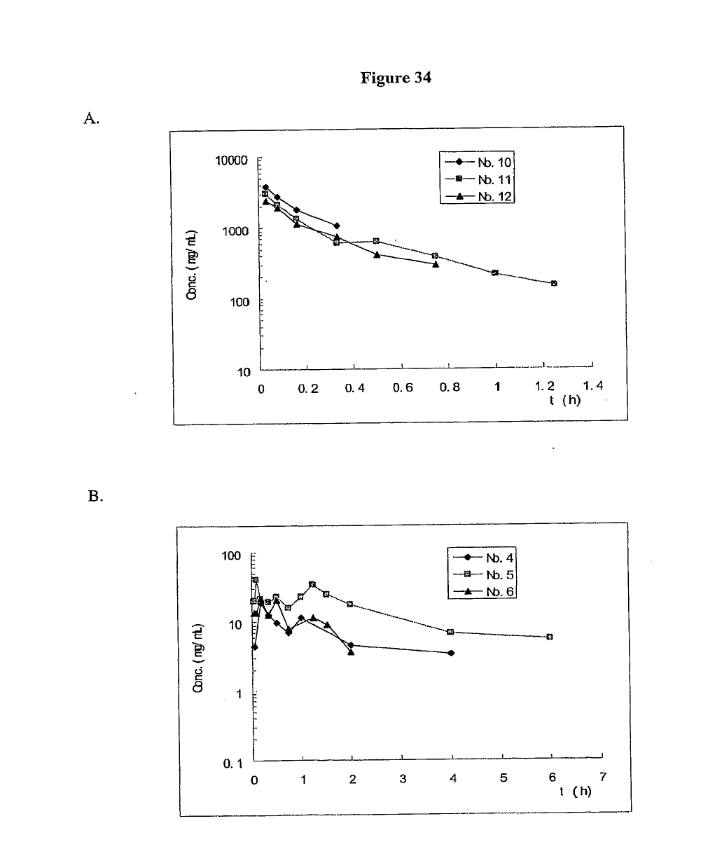

To evaluate the pharmacokinetics of ABS212, plasma samples were collected from Sprague-Dawley (SD) rats at various time points following intravenous and oral administration of test article ABS212. These samples were used for the determination of plasma parent drug concentrations by LC/MS/MS for estimating pharmacokinetic parameters and oral bioavailability. The concentration versus time curve for IV and oral administration of ABS212 are shown in FIGS. 34 A and B.

Following IV bolus injection of ABS-212.2HCL at 5, 2.5 and 1 mg/kg, the mean±SD values of systemic clearance were 0.40±0.05, 0.40±0.17 and 1.07±0.15 L/hr/kg, which corresponded to 12.08%, 12.08% and 32.33% of rat hepatic blood flow (3.31 L/hr/kg), respectively. The mean±SD values of half-life (T_{1/2}) for ABS-212.2HCL were 0.29±0.13, 0.28±0.11 and 0.28±0.08 hr. ABS-212.2HCL dose not distributes well into the tissues. The volume of distribution at terminal phase following IV administration at nominal doses of 5, 2.5 and 1 mg/kg were 0.16±0.06, 0.15±0.01 and 0.43±0.15 L/kg, respectively, which were less than the total body water (0.67 L/kg) in the rats. The dose-normalized AUC_{(0-∞)} was 2.68:2.92:1 at respective doses of 5, 2.5, and 1 mg/kg, while the dose-normalized Cₘₐₓ was 2.68:2.46:1 at doses of 5, 2.5, and 1 mg/kg, respectively, suggesting there was a saturable elimination when given high dosage.

Following oral administration of ABS-212.2HCL at a dose of 50 mg/kg, the mean±SD values of Cₘₐₓ and Tₘₐₓ for ABS-212.2HCL were 27.08±11.55 μg/L and 0.14±0.05 hr, respectively; the mean±SD values of AUC_{(0-∞)} and half-life (T_{1/2}) were 53.06±39.65 hr*μg/L and 1.42±0.87 hr, respectively. The mean±SD value of bioavailability for ABS-212.2HCL was 0.11±0.08% with choice of 1 mg/kg group as IV dose data.

### LIST OF DOCUMENTS

The following list of documents provide background information, synthetic information, scientific information, protocols and related disclosures. The complete text of each document is incorporated herein as an integral part of this application as if it were fully repeated, and all publications, patents and patent applications cited herein are herein incorporated by reference.
(1) Rowley, M., Bristow, L. J., and Hutson, P. H., “Current and novel approaches to the drug treatment of schizophrenia.” J Med. Chem. 44: 477-501, 2001.
(2) Kitabgi, P. and Nemeroff, C. B. Eds. “The Neurobiology of Neurotensin.” Ann NY Acad. Sci. 668: 1-374, 1992.
(3) Kapur, S., Remington, G., Jones, C., Wilson, A., DaSilva, J., Houle, S., and Zipursky, R. “High levels of dopamine D₂ receptor occupancy with low dose haloperidol treatment: a PET study.” Am J Psych. 153: 948-50, 1996.
(4) Alvir, J. M. J., Lieberman, J. A., Safferman, A. Z., Schwimmer, J. L., and Schaff, J. A. “Clozapine-induced agranulocytosis: incidence and risk factors in the United States.” New Eng J Med. 329: 162, 1993.
(5) Andreasen, N. C., Arndt, S., Swayze, V., Cizadlo, T., Flaum, M., Oleary, D., Ehrhardt, J C., and Yuh, W. T. C. “Thalamic abnormalities in schizophrenia visualized through magnetic resonance imaging averaging.” Science. 266: 294-98, 1994.
(6) Lawrie, S. M. and Abukmeil, S. S. “Brain abnormality in schizophrenia. A systematic and quantitative review of volumetric magnetic resonance imaging studies.” Br J Psych. 55: 433-40, 1998.
(7) Silberweig, D. A., Stem, E., Frith, C., Cahill, C., Holmes, A., Grootoonk, S., Seaward, J., McKenna, P., Chua, S. E., and Schnorr, L. “A functional neuroanatomy of hallucinations in schizophrenia” Nature. 378: 176-79, 1995.
(8) McGaffin, P. Owen, M. J., and Farmer, A. E. “Genetic basis of schizophrenia.” Lancet. 346: 678-82, 1995.
(9) Carlsson, A. and Lindquist, M. “Effect of chlorpromazine and haloperidol on formation of methoxytyramine and normetanephrine in mouse brain.” Acta Pharmacol Toxicol. 20: 140-44, 1963.
(10) Creese, I., Burt, D. R., and Snyder, S. H. “Dopamine receptor binding predicts clinical and pharmacological potencies of antischizophrenic drugs.” Science. 192: 481-83, 1976.
(11) Nyberg, S., Nakashima, Y., Nordstrom, A. L., Hallidin, C., and Farde, L. “Positron emission tomography of in vivo binding characteristics of atypical antipsychotic drugs: review of D₂ and 5-HT₂ receptor occupancy studies and clinical response.” Br J Psych. 168 (suppl 29): 40-44, 1996.
(12) Carraway, R. and Leeman, S. E “The isolation of a new hypotensive peptide, neurotensin, from bovine hypothalami.” J Biol Chem. 248: 6854-61, 1973.
(13) Bissette, G., Nemeroff, C. B., Loosen, P. T., Prange Jr., A. J., and Lipton, M A. “Hypothermia and intolerance to cold induced by intracisternal administration of the hypothalamic peptide neurotensin.” Nature. 262: 607-9, 1976.
(14) Nemeroff, C. B., Osbahr, A. J., Manberg, P. J., Ervin, G. N., and Prange Jr., A J. “Alterations in nociception and body temperature after intracisternal administration of neurotensin, β-endorphin, other endogenous peptides, and morphine.” Proc Natl Acad Sci USA. 76: 5368-71, 1979.
(15) Skoog, K. M., Cain, S. T., and Nemeroff, C B. “Centrally administered neurotensin suppresses locomotor hyperactivity induced by d-amphetamine but not by scopolamine or caffeine.” Neuropharm. 25:777-82, 1986.
(16) Nemeroff, C. B., Bissette, G., Prange Jr., A. J., Loosen, P. T., Barlow, T. S., and Lipton, M. A. “Neurotensin: central nervous system effects of a hypothalamic peptide.” Brain Res. 128: 485-96, 1977.
(17) Kitabgi, P., Checker, F., Mazella, J., and Vincent, J. P. “Pharmacology and biochemistry of neurotensin receptors.” Rev Clin Basic Pharmacol. 5: 397-486, 1985.
(18) Carraway, R. and Leeman, S E. “The amino acid sequence of a hypothalamic peptide, neurotensin.” J Biol Chem. 250: 1907-11, 1975.
(19) Carraway, R. and Leeman, S E. “Structural requirements for the biological activity of neurotensin, a new vasoactive peptide.” Edited by R. Walter and J. Meienhofer. Peptides: Chemistry, Structure, and Biology. Ann Arbor: Ann Arbor Science, 1975, p. 679-85.
(20) Tanaka, K., Masu, M., and Nakanishi, S. “Structure and functional expression of the cloned rat neurotensin receptor.” Neuron. 4: 847-54, 1990.
(21) Vita, N., Laurent, P., Lefort, S., Chalon, P., Dumont, X., Kaghad, M., Gully, D., Le Fur, G., Ferrara, P., and Caput, D. “Cloning and expression of a complementary DNA encoding a high affinity human neurotensin receptor.” FEBS Lett. 317: 139-42, 1993.
(22) Hermans, E. and Maloteaux, J. “Mechanisms of regulation of neurotensin receptors.” Pharmacol Ther. 79: 89-104, 1998.
(23) Vincent, J. P. “Neurotensin receptors: binding properties, transduction pathways, and structure.” Cell Mol Neurobiol. 15: 501-12, 1995.
(24) Chalon, P., Vita, N., Kaghad, M., Guillemot, M., Bonnin, J., Delpech, B., Le Fur, G., Ferrara, P., and Caput, D. “Molecular cloning of a levocabastine-sensitive neurotensin binding site.” FEBS Lett. 386: 91-4, 1996.
(25) Vita, N., Oury-Donat, F., Chalon, P., Guillemot, M., Kaghad, M., Bachy, A., Thurneyssen, O., Garcia, S., Poinot-Chazel, C., Casellas, P., Keane, P., Le Fur, G., Maffrand, J. P., Soubrie, P., Caput, D., and Ferrara, P. “Neurotensin is an antagonist of the human neurotensin NT2 receptor expressed in Chinese hamster ovary cells.” Eur J Pharmacol. 360: 265-72, 1998.
(26) Vincent, J. P., Mazella, J., and Kitabgi, P. “Neurotensin and neurotensin receptors.” Trends Pharm Sci. 20: 302-9, 1999.
(27) Mazella, J., Zsurger, N., Navarro, V., Chabry, J., Kaghad, M., Caput, D., Ferrara, P., Vita, N., Gully, D., Maffrand, J. P., and Vincent, J. P. “The 100 k-Da neurotensin receptor is gp95/sortilin, a non-G-protein-coupled receptor.” J Biol Chem. 273: 26273-6, 1998.
(28) Binder, E. B., Kinkead, B., Owens, M. J., and Nemeroff, C. B. “The role of neurotensin in the pathophysiology of action of antipsychotic drugs.” Biol Psych. 50: 856-72, 2001.
(29) Binder, E. B., Kinkead, B., Owens, M. J., and Nemeroff, C. B. “Neurotensin and dopamine interactions.” Pharm Reviews. 53: 453-86, 2001.
(30) Quirion, R., Rowe, W. B., Lapchak, P. A., Araujo, D. M., and Beaudet, A. “Distribution of neurotensin receptors in mammalian brain. What it is telling us about its interactions with other neurotransmitter systems.” Ann NY Acad Sci. 668: 109-19, 1992.
(31) Kinkead, B., Binder, E. B., and Nemeroff, C. B. “Does neurotensin mediate the effects of antipsychotic drugs?” Biol Psychiatry. 46: 340-51, 1999.
(32) Garver, D. L., Bissette, G., Yao, J. K., and Nemeroff, C. B. “Relation of CSF neurotensin concentrations to symptoms and drug response of psychotic patients.” Am J Psych. 148: 484-88, 1991.
(33) Breslin, N. A., Suddath, R. L., Bissette, G., Nemeroff, C. B., Lowrimore, P., and Weinberger, D. R. “CSF concentrations of neurotensin in schizophrenia: An investigation of clinical and biochemical correlates.” Schizophr Res. 12: 35-41, 1994.
(34) Sharma, R. P., Janicak, P. G., Bissette, G., and Nemeroff, C. B. “CSF neurotensin concentrations and antipsychotic treatment in schizophrenia and schizoaffective disorder.” Am J Psych. 154: 1019-21, 1997.
(35) Kitabgi, P., Poustis, C., Granier, C., Van Rietschoten, J., Rivier, J., Morgat, J. L., and Freychet, P. “Neurotensin binding to extraneural and neural receptors: comparison with biological activity and structure-activity relationships.” Mol Pharmacol. 18: 11-19, 1980.
(36) Fuxe, K., Von Euler, G., Agnati, L. F., Merlo Pich, E., O'Connor, W. T., Tanganelli, S., et al. “Intermembrane interactions between neurotensin receptors and dopamine D₂ receptors as a major mechanism for the neuroleptic-like action of neurotensin.” Ann NY Acad Sci. 668: 186-204, 1992.
(37) Li, X. M., Ferraro, L., Tanganelli, S., O'Connor, W. T., Hasselrot, U., Ungerstedt, U., et al., “Neurotensin peptides antagonistically regulate postsynaptic dopamine D₂ receptors in rat nucleus accumbens: A receptor binding and microdialysis study.” J Neural Trans. 102: 125-37, 1995.
(38) Binder, E. B., Kinkead, B., Owens, M. J., Kilts, C. D., and Nemeroff, C. B. “Enhanced neurotensin neurotransmission is involved in the clinically relevant behavioral effects of antipsychotic drugs: evidence from animal models of sensorimotor gating.” J Neurosci. 21: 601-8, 2001.
(39) Al-Rodhan, N. R., Richelson, E., Gilbert, J. A., McCormick, D. J., Kanba, K. S. Pfenning, M. A., Nelson, A., Larson, E. W., and Yaksh, T. L. “Structure-antinociceptive activity of neurotensin and some novel analogues in the periaqueductal gray region of the brainstem.” Brain Res. 557(1-2): 227-35, 1991.
(40) Pettibone, D. J., Hess, J. F., Hey, P. J., Jacobson, M. A., Leviten, M., Lis, E. V., Mallorga, P. J., Pascarella, O. M., Snyder, M. A., Williams, J. B., and Zeng, Z. “The effects of deleting the mouse neurotensin receptor NTR1 on central and peripheral responses to neurotensin.” J Pharm Exp Ther. 300: 305-313, 2001.
(41) Tyler-McMahon, B. M., Stewart, J. A., Farinas, F., McCormick, D. J., and Richelson, E. “Highly potent neurotensin analog that causes hypothermia and antinociception.” Eur J Pharmacol. 390: 107-11, 2000.
(42) Farra, C. D., Sarret, P., Navarro, V., Botto, J. M., Mazella, J., and Vincent, J. P. “Involvement of the neurotensin receptor subtype NTR3 in the growth effect of neurotensin on cancer cell lines.” Int J Cancer. 92: 503-9, 2001.
(43) Moody, T. W., Chiles, J., Casibang, M., Moody, E., Chan, D., and Davis, T. P. “SR48692 is a neurotensin receptor antagonist which inhibits the growth of small cell lung cancer cells.” Peptides. 22: 109-15, 2001.
(44) Iwase, K., Evers, B. M., Hellmich, M, R., Kim, H. J., Higashide, S., Gully, D., Thompson, J. C., and Townsend Jr., C. M. “Inhibition of neurotensin-induced pancreatic cancer growth by a nonpeptide neurotensin receptor antagonist, SR48692.” Cancer. 79: 1787-93, 1997.
(45) Schimpff, R. M., Avard, C., Fenelon, G., Lhiaubet, A. M., Tenneze, L., Vidailbet, M., and Rostene, W. “Increased plasma neurotensin concentrations in patients with Parkinson's disease.” J Neurol Neuro Psych. 70: 784-6, 2001.
(46) Boules, M., Warrington, L., Fauq, A., McCormick, D., and Richelson, E. “Antiparkinson-like effects of a novel neurotensin analog in unilaterally 6-hydroxydopamine lesioned rats.” Eur J Pharmacol. 428: 227-33, 2001.
(47) Beeson, C. C. and Dix, T. A. “Thermodynamic description of a contact and solvent-separated ion pair as a function of solvation: A model for salt bridges and proton-transfer reactions in biology.” J Am Chem Soc. 115: 10275-81, 1993.
(48) Kennedy, K. J., Lundquist, J. T., Simandan, T. L., Kokko, K. P., Beeson, C. C., and Dix, T. A. “Design rational, synthesis, and characterization of non-natural analogs of the cationic amino acids arginine and lysine.” J Pept Res. 55: 348-58, 2000.
(49) Cusack, B., McCormick, D. J., Pang, Y. P., Sonder, T., Garcia, R., Fauq, A., and Richelson, E. “Pharmacological and biochemical profiles of unique neurotensin 8-13 analogs exhibiting species selectivity, stereoselectivity, and superagonism.” J Biol Chem. 270: 18359-66, 1995.
(50) Lundquist, J. T. and Dix, T. A. “Synthesis and human neurotensin receptor binding activities of neurotensin(8-13) analogues containing position 8 alpha-azido-N-alkylated derivatives of ornithine, lysine, and homolysin.” J Med Chem. 42: 4914-8, 1999.
(51) Lee, Y. C., Uttenthal, L. O., Smith, H. A., and Bloom, S. R. “In vitro degradation of neurotensin in human plasma.” Peptides. 7: 383-7, 1986.
(52) Kokko, K. P., Hadden, M. K., Price, K. L., Orwig, K. S., See, R. E., and Dix, T A. “Pharmacokinetic and behavioral effects of stable, receptor-selective neurotensin(8-13) analogues that cross the blood-brain barrier.” Neuropharm. Under Revision.
(53) Kokko, K. P. and Dix, T A. “Monitoring neurotensin [8-13] degradation in human and rat serum utilizing matrix-assisted laser desorption/ionization time-of-flight mass spectrometry.” Anal Biochem. 308: 34-41, 2002.
(54) Kokko, K. P., Hadden, M. K., Orwig, K. S., Mazella, J., and Dix, T. A. “In vitro analysis of stable, receptor-selective neurotensin(8-13) analogues.” J Med Chem. 46: 4141-48, 2003.
(55) Tyler-McMahon, B. M., Boules, M., and Richelson, E. “Neurotensin: peptide for the next millennium.” Reg Pept. 93: 125-36, 2000.
(56) Bednarek, M. A., Macneil, T., Kalyani, R. N., Tang, R., Van der Ploeg, L. H. T., and Weinberg, D. H. “Analogs of MTII, lactam derivatives of α-melanotropin, modified at the N-terminus, and their selectivity at human melanocortin receptors 3, 4, and 5.” Biochem Biophys Res Comm. 261: 209-13, 1999.
(57) Lunquist, J. T. and Dix, T. A. “Asymmetric synthesis of ω-bromo-2(S)-azido acids as precursors for the synthesis of novel amino acids.” Tet Lett. 39: 775-8, 1998.
(58) Evans, D. A., Britton, T. C., Ellman, J. A., and Dorow, R. L. “The asymmetric synthesis of α-amino acids. Electrophilic azidation of chiral imide enolates, a practical approach to the synthesis of (R)- and (S)-α-azido carboxylic acids.” J Am Chem. Soc. 112: 4011-30, 1990.
(59) Taylor, R. E., Galvin, G. M., Hilfiker, K. A., and Chen, Y. “A formal total synthesis of epothilone A: Enantioselective preparation of the C1-C6 and C7-C12 fragments.” J Org Chem. 63: 9580-3, 1998.
(60) Boules, M., McMahon, B., Warrington, L., Stewart, J., Jackson, J., Fauq A., McCormick, D., and Richelson, E. “Neurotensin analog selective for hypothermia over antinociception and exhibiting atypical neuroleptic-like properties.” Brain Res. 919: 1-11, 2001.
(61) Dubuc, I., Remande, S., and Costentin, J. “The partial agonist properties of levocabastine in neurotensin-induced analgesia.” Eur J Pharmacol. 381: 9-12, 1999.
(62) Kennedy, K. J., Simandan, T. L., and Dix, T A. “A facile route to cyclic and acyclic alkyl-arginines.” Synthetic Comm. 28: 741-6, 1998.
(63) Lundquist, J. T., Orwig, K. S., and Dix, T. A “Synthesis of ethylene-bridge (N^{δ} to N^{ω}) analogues of arginine.” J Org Chem. 64:9265-7, 1999.
(64) Lundquist, J. T., and Pelletier, J. C. “Improved solid-phase peptide synthesis method utilizing alpha-azide protected amino acids.” Org. Lett. 3: 781-3, 2001.
(65) Meienhofer, J., Waki, M., Heimer, E. P., Lambros, T. J., Makofske, R., and Chang, C. C. “Solid phase synthesis without repetitive acidolysis; preparation of leucyl-alanyl-glycyl-valine using 9-fluorenylmethyloxylcarbonyl amino acids.” Int J Pep Pro Res. 13: 35-42, 1979.
(66) Kaiser, E., Colescott, R. L., Bossinger, C. D., and Cook, P. I. “Color test for detection of free terminal amino groups in the solid-phase synthesis of peptides.” Anal Biochem. 34: 595-98, 1970.
(67) Boules, M., Warrington, L., Fauq, A., McCormick, D., and Richelson, E. “A novel neurotensin analog blocks cocaine- and D-amphetamine-induced hyperactivity.” Eur J Pharmacol. 426: 73-6, 2001.
(68) Tyler, B. M., Douglac, C. L., Faur, A., Pang, Y. P., Stewart, J. A., Cusack, B., McCormick, D. J., and Richelson, E. “In vitro binding and CNS effects of novel neurotensin agonists that cross the blood-brain barrier.” Neuropharm. 38: 027-34, 1999.
(69) Richelson, E., Cusack, B., Pang, Y. P., McCormick, D. J., Fauq, A., Tyler, B. M., and Boules, M. U.S. Pat. No. 6,214,790. United States Patent and Trademark Office, Mayo Foundation for Medical Education and Research, USA, 2000.
(70) Machida, R., Tokumura, T., Tsuchiya, Y., Sasaki, A., and Abe, K. “Pharmacokinetics of a novel hexapeptide with neurotensin activity in rats.” Biol Pharm Bull. 16: 43-7, 1993.
(71) Ogren, S. O. “The behavioural pharmacology of typical and atypical antipsychotic drugs.” Handbook of Experimental Pharmacology. Edited by J. G. Csernansky, Springer, Berlin, 1996, p. 225-66.
(72) Jones, G. H. and Robbins, T. W. “Differential effects of mesocortical, mesolimbic, and mesostriatal dopamine depletion on spontaneous, conditioned and drug-induced locomotor activity.” Pharmacol Biochem Behay. 43: 887-95, 1992.
(73) Hertel, P., Byskov, L., Didriksen, M., and Amt, J. “Induction of tolerance to the suppressant effect of the neurotensin analogue NT69L on amphetamine-induced hyperactivity.” Eur J Pharmacol. 422: 77-81, 2001.
(74) Lowe, J. A., Seeger, T. F., and Vineck F. J. “Atypical antipsychotics—recent findings and new perspectives.” Med Res Rev. 8: 475-97, 1998.
(75) Dunn, A. J., Snijders, R., Hurd, R. W., and Kramery, N. R. “Induction of catalepsy by central nervous system administration of neurotensin.” Ann NY Acad Sci. 400: 345-53, 1982.
(76) Sarhan, S., Hitchcock, J. M., Grauffel, C. A., and Wettstein, J. G. “Comparative antipsychotic profiles of neurotensin and a related systemically active peptide agonist.” Peptides. 18: 1223-27, 1997.
(77) Cusack, B., Boules, M., Tyler, B. M., Fauq, A., McCormick, D. J., and Richelson, E. “Effects of a novel neurotensin peptide analog given extracranially on CNS behaviors mediated by apomorphine and haloperidol.” Brain Res. 865: 48-54, 2000.
(78) Hilgers, A. R., Conradi, R. A., and Burton, P. S. “Caco-2 cell monolayers as a model for drug transport across the intestinal mucosa.” Pharm Res. 7: 902-10, 1990.
(79) Artursson, P. and Karisson, J: “Correlation between drug absorption in humans and apparent drug permeability coefficients in human intestinal epithelial (Caco-2) cells.” Biochem Biophy Res Comm. 175: 880-5, 1991.
(80) Borchardt, R. T. “Optimizing oral absorption of peptides using prodrug strategies.” J Cont Rel. 62: 231-38, 1999.
(81) Conradi, R. A., Hilgers, A. R., Ho, N. F. H., and Burton, P. S. “The influence of peptide structure on transport across Caco-2 cells.” Pharm Res. 8: 1453-60, 1991.
(82) McGuill, M. W. and Rowan, A. N. “Biological effects of blood loss; implication for sampling volumes and techniques.” ILAR Journal. 31(4), 1989.
(83) Lowry, O. H., Rosenbrough, N. J., Farr, A. L., and Randall, R. J. “Protein measurement with the folin phenol reagent.” J Biol Chem. 193: 265-75, 1951.
(84) Hadjiagapiou, C., Schmidt, L., Dudeja, K., Layden, T J., and Ramaswamy, K. “Mechanism(s) of butyrate transport in Caco-2 cells: role of monocarboxylate transporter 1.” Am J Physiol. 279: G775-80, 2000.
(85) Brandsch, M., Miyamoto, Y., Ganaphthy, V., and Liebach, F. “Expression of protein kinase C-dependent regulation of peptide/H⁺ co-transport system in the Caco-2 human carcinoma cell line.” Biochem J. 299: 253-60, 1994.
(86) Garcia, C. K., Goldstein, J. L., Pathak, R. K., Anderson, R. G. W., and Brown, M. S. “Molecular characterization of a membrane transporter for lactate, pyruvate and other monocarboxylates: implications for the con cycle.” Cell. 76: 865-73, 1994.
(87) Tamai, I., Sai, Y., Ono, A., Kido, Y., Yabuuchi, H., Takanaga, H., Satoh, E., Ogihara, T., Amano, O., Izeki, S., and Tsuji, S. “Immunohistochemical and functional characterization of pH-dependent intestinal transport of weak organic acids by the monocarboxylic acid transporter MCTI.” J Pharm Pharmacol. 51: 1113-21, 1999.
(88) Tamai, I., Onashi, R., Nezu, J., Yabuuchi, H., Oku, A., Shimane, M., Sai, Y., and Tsuji, A. “Molecular and functional identification of sodium ion-dependent, high affinity human carnitine transporter OCTN2.” J Biol Chem. 273: 20378-82, 1998.
(89) Walgren R. A., Karnaky, K. J., Lindenmayer, G. E., and Walle, T. “Efflux of dietary flavonoid quercetin 4′-β-glucoside across human intestinal Caco-2 cell monolayers by apical multidrug resistance-associated protein-2.” J Pharm Exp Ther. 294:830-6, 2000.
(90) Hunter, J., Jepson, M. A., Tsuruo, T., Simmons, N. L., and Hirst, B. H. “Functional expression of P-glycoprotein in apical membranes of human intestinal Caco-2 cells. Kinetics of vinblastine secretion and interaction with modulators.” J Biol Chem. 268: 14991-7, 1993.
(91) Jedlitschky, G., Leier, I., Buchholz, U., Center, M., and Keppler, D. “ATP-dependent transport of glutathione S-conjugates by the multidrug-resistance-associted protein.” Cancer Res. 54: 4833-36, 1994.
(92) Bohme, M., Buchler, M., Muller, M., and Keppler, D. “Differential inhibition by cyclosporins of primary-active ATP-dependent transporters in the hepatocyte canalicular membranes.” FEBS Lett. 333: 193-6, 1993.
(93) Hayer-Zillgen, M., Bruss, M., and Bonisch, H. “Expression and pharmacological profile of the human organic cation transporters hOCT1, hOCT2, and hOCT3.” Br J Pharmacol. 136: 829-36, 2002.
(94) Grundemann, D., Liebich, G., Kiefer, N., Koster, S., and Schomig, E. “Selective substrates for non-neuronal monoamine transporters.” Mol Pharmacol. 56; 1-10, 1999.
(95) Mentlein, R. and Dahms, P. “Endopeptidases 24.16 and 24.15 are responsible for the egradation of somatostatin, neurotensin, and other neuropeptides by cultivated rat cortical astrocytes.” J Neurochem. 62; 27-36, 1994.
(96) Lipinski, C. A., Lombardo, F., Dominy, B. W., Feeney, P. J., “Experimental and computational approaches to estimate solubility and permeability in drug discovery and development settings.” Adv Drug Del Rev. 46: 3-26, 2001.
(97) Kilts, C. D. “Animal behavioral models of schizophrenia.” Current Issues in the Psychology of Schizophrenia, edited by A. Breier. Lippincott Williams and Wilkins, 2001, p. 111-130.
(98) Material in this section abstracted from “Schizophrenia” NIH Publication No. 99-3517.
(99) Lieberman, J. A, et. al (2005) Effectiveness of antipsychotic drugs in patients with chronic schizophrenia. New Engl. J. Med. 353, 1209-1222.
(100) The Boston Globe, Sep. 20, 2005.
(101) Vincent J.-P.; Mazella, J.; and Kitabgi, P. (1999) Neurotensin and neurotensin receptors. Trends. Pharmacol. Sci. 20, 302-309.
(102) Carraway, R. and S. E. Leeman, (1975) The amino acid sequence of a hypothalamic peptide, neurotensin. J. Biol. Chem. 259, 1907-1913.
(103) Nemeroff, C. B. (1980) Neurotensin: perchance an endogenous neuroleptic? Biol. Psychiatry, 15: 283-302.
(104) Benzing, W. C.; Mufson, E. J.; Jennes, L.; and Armstrong, D. M. (1990) Reduction of neurotensin immunoreactivity in the amygdala in Alzheimer's disease. Brain Res. 537: 298-302.
(105) Govoni, S.; Hong, J. S.; Yang, H. Y.; and Costa, E. (1980) Increase of neurotensin content elicited by neuroleptics in nucleus accumbens. J. Pharmacol. Exp. Ther., 215: 413-417.
(106) Widerlov, E.; Lindstrom, L. H.; Besev, G.; Manberg, P. J.; Nemeroff, C. B.; Breese, G. R.; Kizer, J. S.; and Prange Jr., A. J. (1982) Subnormal CSF levels of neurotensin in a subgroup of schizophrenic patients: normalization after neuroleptic treatment. Am. J. Psychiatry, 139: 1122-1126.
(107) Garver, D. L.; Bissette, G.; Yao, J. K.; and Nemeroff, C. B. (1991) Relation of CSF neurotensin concentrations to symptoms and drug response of psychotic patients. Am. J. Psychiatry, 148: 484-488.
(108) Baldessarini, R. J. (1996) “Drugs and the treatment of psychiatric disorders: psychosis and anxiety.” Hardman, J. G.; and Limbird, L. E., eds. in Goodman and Gilman's The Pharmacological Basis of Therapeutics, 9^{th} ed. 399-420. 28.
(109) Kinkead B.; Binder E B. and Nemeroff C B. (1999) Does neurotensin mediate the effects of antipsychotic drugs? Biological Psychiatry. 46, 340-51.
(110) Hadden, M. K.; Kokko, K. P.; Orwig, K.; Mazella, J. and Dix, T. A. (2005) Design, synthesis, and evaluation of the antipsychotic potential of orally bioavailable neurotensin (8-13) analogues containing non-natural arginine and lysine residues. Neuropharmacology 49, 1149-1159.
(111) Orwig, K. (2005) Ph.D. dissertation, Medical University of South Carolina.
(112) Ogren S. O. (1996) The behavioral pharmacology of typical and atypical antipsychotic drugs, in Handbood of Experimental Pharmacology (Csemansky J. G. ed.) pp 225-266, Springer, Berlin.
(113) Hitzemann, R. (2000) Animal models of psychiatric disorders and their relevance. Alcohol Res. And Health 24, 149-158.
(114) Feifel, D.; Melendez, G. and Shilling, P. D. (2004) Reversal of sensorimotor gating deficits in Brattleboro rats by acute administration of clozapine and a neurotensin agonist, but not haloperidol: a potential predictive model for novel antipsychotic effects. Neuropsychopharmacology 29, 731-738.
(115) MacPherson, R. D., The pharmacological basis of contemporary pain management. Pharmacology & Therapeutics, 2000. 88(2): p. 163.
(116) Smith, B. H., et al., The impact of chronic pain in the community. Family Practice, 2001. 18(3): p. 292.
(117) Nitu, A. N., et al., Emerging trends in the pharmacotherapy of chronic pain. [Review][101 refs]. (1354-3784).
(118) Jones, S. C., Relative Thromboembolic Risks Associated with COX-2 Inhibitors. The Annals of Pharmacotherapy, 2005. 39(7): p. 1249.
(119) Garber, K., Peptides lead new class of pain drugs. Nature Biotech., 2005. 23: p. 399.
(120) Carraway, R. and S. E. Leeman, The Isolation of a New Hypotensive Peptide, Neurotensin, from Bovine Hypothalami. Journal of Biological Chemistry, 1973. 248(19): p. 6854.
(121) Carraway, R. and S. E. Leeman, The amino acid sequence of a hypothalamic peptide, neurotensin. Journal of Biological Chemistry, 1975. 250(5): p. 1907.
(122) Chapman, C. R., et al., Pain measurement: an overview. Pain, 1985. 22(1): p. 1.
(123) Le Bars, D., M. Gozariu, and S. W. Cadden, Animal Models of Nociception. Pharmacological Reviews, 2001. 53(4): p. 597.
(124) Mogil, J. S. and S. E. Crager, What should we be measuring in behavioral studies of chronic pain in animals? Pain, 2004. 112(1-2): p. 12.
(125) Gracely, R. H., Pain Measurement. Acta Anaesthesiol. Scand., 1999. 43: p. 897-908.
(126) Mogil, J. S., B. Kest, B. Sadowski, and J. K. Belknap Differential Genetic Mediation to morphine in genetic models of opiate antinociception: Influence of nociceptive assay. J. Pharmacol. Exp. Ther. 1996, 276, 532-544.
(127) Kim S. H. and Chung J. M. An experimental model for peripheral neuropathy produced by segmental spinal nerve ligation in the rat. Pain 50: 355-363.

Additional advantages of the invention in part will be obvious from the description, or may be learned by practice of the invention. The advantages of the invention will be realized and attained by means of the elements and combinations particularly pointed out in the appended claims.

Throughout this application, where publications are referenced, the disclosures of these publications in their entireties are hereby incorporated by reference into this application in order to more fully describe the state of the art to which this invention pertains.

1. A non-natural desamino, alkyl amino acid compound having a formula selected from the group consisting of Formulas I, II, III, IV, and V, wherein:
(a) formula I is 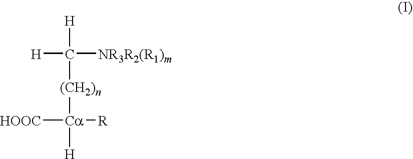
and wherein
n is an integer of from 0 to 5;
m is zero or an integer of 1;
R is H;
R₁, R₂, and R₃ are, independently, hydrogen or branched or straight chain alkyl, alkenyl or alkynyl of C₁-C₆, a protecting group that is removable by a chemical method that does not also cause cleavage of other groups, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination and with the proviso that a maximum of two of R₁, R₂, and R₃ may be selected to be the aromatic, substituted aromatic, heteroaromatic or substituted heteroaromatic group, and provided that when m is 0 or 1 and n is 0 to 5, R₁, R₂, and R₃ are not all H;
and Cα is a carbon atom having either R or S stereochemistry;
or an ester, amide, alkyl amide or metal cation or ammonium salt of the carboxylic acid group thereof, or an organic or inorganic acid salt of the amine group thereof, or any combination thereof;
(b) Formula II is: 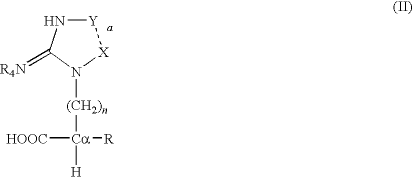
and wherein
n is an integer of from 0 to 6;
when dashed line a is not present, X and Y are independently, hydrogen or lower branched or straight chain alkyl, alkenyl or alkynyl of C₁-C₆;
when dashed line a is present, X—Y is (CH₂)_{z}, wherein z is an integer of from 1-8;
R is H;
R₄ is hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination, and;
Cα is a carbon atom and the stereochemistry at Cα, is either R or S;
or an ester, amide, alkyl amide or metal cation or ammonium salt of the carboxylic acid group thereof, or an organic or inorganic acid salt of the amine group thereof, or any combination thereof;
(c) formula III is: 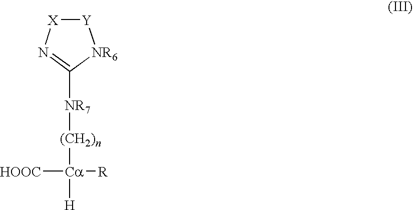
and wherein
n is an integer of from 0 to 5;
X—Y is (CH₂)_{z}, wherein z is an integer of from 0 to 6;
R is H;
R₆, and R₇ are, independently, hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination; and
Cα is a carbon atom and the stereochemistry at Cα, is either R or S;
or an ester, amide, alkyl amide or metal cation or ammonium salt of the carboxylic acid group thereof, or an organic or inorganic acid salt of the amine group thereof, or any combination thereof;
(d) Formula IV is: 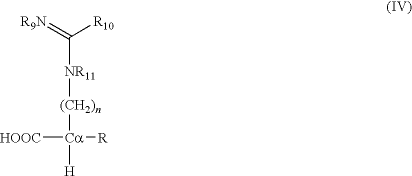
and wherein
n is an integer of from 0 to 5;
R is H;
R₉, R₁₀, and R₁₁ are, independently, hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination and with the proviso that a maximum of two of R₉, R₁₀, and R₁₁ may be selected to be the aromatic, substituted aromatic, heteroaromatic or substituted heteroaromatic group; and
Cα is a carbon atom and the stereochemistry at Cα, is either R or S;
or an ester, amide, alkyl amide or metal cation or ammonium salt of the carboxylic acid group thereof, or an organic or inorganic acid salt of the amine group thereof, or any combination thereof;
and
(e) formula V is: 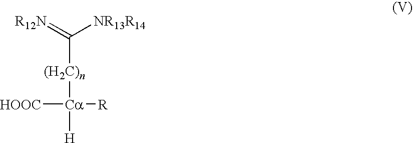
n is an integer of from 0 to 5;
R is H; and
R₁₂, R₁₃, and R₁₄ are, independently, hydrogen or lower branched or straight chain alkyl, alkenyl or alkynyl of C₁-C₆, or an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination and with the proviso that a maximum of two of R₁₂, R₁₃, and R₁₄ may be selected to be the aromatic, substituted aromatic, heteroaromatic or substituted heteroaromatic group; and
or an ester, amide, alkyl amide or metal cation or ammonium salt of the carboxylic acid group thereof, or an organic or inorganic acid salt of the amine group thereof, or any combination thereof;
further wherein the carboxy group of any of compounds I, II, III, IV, or V may be protected by a second protecting group that is removable by a chemical method that does not also cause cleavage of other groups.

2. The compound of claim 1, wherein the compound is a compound of formula V and the stereochemistry at Cα is S;

3. The compound of claim 1, wherein the compound is a compound of formula I and R₁, R₂, and R₃ are, independently, hydrogen or methyl.

4. The compound of claim 1, wherein the compound is a compound of formula I and n is an integer from 2 to 5.

5. (canceled)

6. The compound of claim 1 wherein the protecting group is selected from the group consisting of BOC (t-butoxy carbonyl), FMOC (fluorenylmethoxycarbonyl), Alloc (allyloxycarbonyl), CBZ (benzyloxycarbonyl), Pbf (2,2,4,6,7-pentamethyldihydrobenzofuran-5-sulfonyl), NO₂ (nitro), Pmc (2,2,5,7,8-pentamethylchroman-6-sulfonyl), Mtr (4-methoxy-2,3,6-trimethylbenzenesulfonyl), and Tos (tosyl).

7. A peptide comprising a residue of at least one amino acid of claim 1 wherein said at least one amino acid is a compound of Formula I and said residue is bound via at least one amide bond.

8. The peptide of claim 7, wherein said peptide is a known peptide, wherein said residue is
covalently coupled through an amide bond to the N-terminus amine group of said known peptide;
a substitute for a corresponding analogous natural amino acid moiety within said known peptide;
or a substitute for a natural amino acid moiety at the N-terminus of said known peptide.

9. The peptide of claim 8, wherein the known peptide is (i) neurotensin (8-13), (ii) a transcription factor, (iii) a ligand for an cellular receptor, (iv) a hormone, (v) an extracellular binding peptide, (vi) ofenkephlin, (vii) LHRH or analogs thereof, (viii) a neuropeptide, (ix) a glycoincretin, (x) an integrin or analogs thereof, (xi) a glucagon, (xii) a glucagon-like peptide, (xiii) an antithrombotic peptide, (xiv) a cytokine, (xv) an interleukin, (xvi) a transferrin, (xvii) an interferon, (xviii) an endothelin, (xix) a natriuretic hormone, (xx) an extracellular kinase ligand, (xxi) an angiotensin enzyme inhibitor, (xxii) a peptide antiviral compound, (xxiii) thrombin, (xxiv) substance P, (xxv) substance G, (xxvi) somatotropin, (xxvii) somatostatin, (xxviii) GnRH or analogues thereof, (xxix) secretin, (xxx) bradykinin, (xxxi) vasopressin or analogues thereof, (xxxii) insulin or analogs thereof, (xxxiii) proinsulin, or (xxxiv) a growth factor.

10. A peptide selected from the group consisting of ABS205, ABS207, ABS208, ABS210, ABS211, ABS212, ABS220, ABS225, ABS226, ABS227, ABS228, ABS230, ABS232, ABS234, and ABS239.

11. The peptide of claim 7 wherein the peptide has an extended half-life in vivo and/or in vitro when compared to a peptide that has the same amino acid sequence as said peptide, but does not comprise a residue of at least one amino acid of claim 1.

12. A pharmaceutical composition comprising the peptide of claim 7 and a pharmaceutical carrier.

13. The pharmaceutical composition of claim 12, wherein the peptide is in unit dosage form.

14. A cosmetic formulation comprising a cosmetic base formulation and (a) a desamino alkyl amino acid compound of formula I, II, III, IV, or V, or (b) the peptide of claim 7.

15. The cosmetic formulation of claim 14, wherein the cosmetic base formulation is an aqueous or oil base.

16. A method of treating a disease comprising administering a compound of formula I, II, III, IV, or V or a peptide of claim 7.

17. A method for manufacturing a medicament comprising a compound of formula I, II, III, IV, or V or a peptide of claim 7 useful for treating psychosis, pain, cancer, obesity, diabetes, or psychostimulant abuse in a mammal.

18. The method of claim 17, wherein the psychosis is schizophrenia.

19. A method of lowering body temperature of a patient comprising administering to the patient an effective amount of a peptide of claim 7.

20. A method of treating cancer comprising administering to a patient an effective amount of a peptide of claim 7.

21. A method of treating pain, comprising administering to a patient an effective amount of a peptide of claim 7.

22. The method of claim 21, wherein the pain is neuropathic pain.

23. A method of treating a patient with psychosis, comprising administering to the patient an effective amount of a peptide of claim 7 so as to treat the psychosis.

24. A method of treating obesity, comprising administering to a patient an effective amount of a peptide of claim 7 so as to treat obesity.

25. A method for screening a peptide for an activity, comprising the steps of: a) measuring a biological activity of a first peptide having a known amino acid sequence, wherein the first peptide does not comprise an amino acid residue of formula I, II, III, IV, or V; and b) measuring the same biological activity of a second the peptide wherein the second peptide has the same amino acid sequence as the first peptide except that the second peptide comprises an amino acid residue of formula I, II, III, IV, or V wherein said residue is
covalently coupled through an amide bond to the N-terminus amine group of said first peptide;
a substitute for a corresponding analogous natural amino acid moiety within said first peptide;
or a substitute for a natural amino acid moiety at the N-terminus of said first peptide.

26. The method of claim 25, wherein the biological activity is selectivity, poptosis, apoptosis, cell signaling, ligand binding, transcription, translation, metabolism, cell growth, cell differentiation, homeostasis, half-life, solubility, transport, or stability.

27. The method of claim 25, wherein the biological activity includes a direct or indirect assessment of the ability of the semisynthetic peptide to pass through a biological barrier.

28. A method of treating a patient with a disease that is affected by administration to the patient of a known first peptide, said method comprising administering to the patient a second peptide comprising an amino acid residue of formula I, II, III, IV, or V wherein the second peptide has the same sequence as the first peptide except that the amino acid residue of formula I, II, III, IV, or V is:
covalently coupled through an amide bond to the N-terminus amine group of said known first peptide:
a substitute for a corresponding analogous natural amino acid moiety within said known first peptide;
or a substitute for a natural amino acid moiety at the N-terminus of said known first peptide.

29. The method of claim 28, wherein the disease is a disease of the brain or wherein the known first peptide crosses a body barrier.

30. A method of increasing the ability of a known peptide to cross a biological barrier, increasing selectivity of a known peptide, or increasing resistance of a known peptide to digestion by a peptidase, comprising substituting for the known peptide a second peptide wherein the second peptide has the same sequence as the known peptide, except that an amino acid residue of formula I, II, III, IV, or V is:
covalently coupled through an amide bond to the N-terminus amine group of said known peptide;
a substitute for a corresponding analogous natural amino acid moiety within said known peptide;
or a substitute for a natural amino acid moiety at the N-terminus of said known peptide.

31. The method of claim 30, wherein the barrier is selected from the group consisting of the blood brain barrier, a cell membrane, intestinal epithelium, skin, and the blood-ocular barrier.

32. The method of claim 31, wherein the barrier is the blood brain barrier.

33. A method for preparing a peptide with an extended half-life in vivo comprising substituting for a known peptide a second peptide wherein the second peptide has the same sequence as the first peptide, except that an amino acid residue of formula I, II, III, IV, or V is:
covalently coupled through an amide bond to the N-terminus amine group of said known peptide;
a substitute for a corresponding analogous natural amino acid moiety within said known peptide;
or a substitute for a natural amino acid moiety at the N-terminus of said known first peptide.

34. The compound of claim 1, wherein the compound is a compound of formula II and n is an integer from 2 to 5 and z is an integer from 2 to 4.

35. The compound of claim 1, wherein the compound is a compound of formula III and n is an integer from 2 to 5, and z is an integer from 2 to 4.

36. The compound of claim 1 wherein the compound is a compound of formula IV and n is an integer from 2 to 4.


SEQUENCE LISTING (58 sequences)

SEQ ID NO: 1 - protein, 6 aa, Artificial Sequence
  completely synthesized amino acid sequence
XRPYXL

SEQ ID NO: 2 - protein, 6 aa, Artificial Sequence
  completely synthesized amino acid sequence
XRPYXL

SEQ ID NO: 3 - protein, 6 aa, Artificial Sequence
  completely synthesized amino acid sequence
XRPYXL

SEQ ID NO: 4 - protein, 6 aa, Artificial Sequence
  completely synthesized amino acid sequence
XRPYXL

SEQ ID NO: 5 - protein, 6 aa, Artificial Sequence
  completely synthesized amino acid sequence
XRPYXL

SEQ ID NO: 6 - protein, 6 aa, Artificial Sequence
  completely synthesized amino acid sequence
XRPYXL

SEQ ID NO: 7 - protein, 6 aa, Artificial Sequence
  completely synthesized amino acid sequence
XRPYXL

SEQ ID NO: 8 - protein, 6 aa, Artificial Sequence
  completely synthesized amino acid sequence
XRPYXL

SEQ ID NO: 9 - protein, 6 aa, Artificial Sequence
  completely synthesized amino acid sequence
XRPYXL

SEQ ID NO: 10 - protein, 6 aa, Artificial Sequence
  completely synthesized amino acid sequence
XRPYIL

SEQ ID NO: 11 - protein, 6 aa, Artificial Sequence
  completely synthesized amino acid sequence
XRPYIL

SEQ ID NO: 12 - protein, 6 aa, Artificial Sequence
  completely synthesized amino acid sequence
XRPYIL

SEQ ID NO: 13 - protein, 6 aa, Artificial Sequence
  completely synthesized amino acid sequence
XRPYIL

SEQ ID NO: 14 - protein, 6 aa, Artificial Sequence
  completely synthesized amino acid sequence
XRPYIL

SEQ ID NO: 15 - protein, 6 aa, Artificial Sequence
  completely synthesized amino acid sequence
XRPYXL

SEQ ID NO: 16 - protein, 6 aa, Artificial Sequence
  completely synthesized amino acid sequence
XRPYXL

SEQ ID NO: 17 - protein, 6 aa, Artificial Sequence
  completely synthesized amino acid sequence
XRPYXL

SEQ ID NO: 18 - protein, 6 aa, Artificial Sequence
  completely synthesized amino acid sequence
XRPYXL

SEQ ID NO: 19 - protein, 6 aa, Artificial Sequence
  completely synthesized amino acid sequence
XRPYXL

SEQ ID NO: 20 - protein, 6 aa, Artificial Sequence
  completely synthesized amino acid sequence
XRPYXL

SEQ ID NO: 21 - protein, 6 aa, Artificial Sequence
  completely synthesized amino acid sequence
XRPYXL

SEQ ID NO: 22 - protein, 6 aa, Artificial Sequence
  completely synthesized amino acid sequence
XRPYXL

SEQ ID NO: 23 - protein, 6 aa, Artificial Sequence
  completely synthesized amino acid sequence
XRPYXL

SEQ ID NO: 24 - protein, 6 aa, Artificial Sequence
  completely synthesized amino acid sequence
XRPYXL

SEQ ID NO: 25 - protein, 6 aa, Artificial Sequence
  completely synthesized amino acid sequence
XRPYXL

SEQ ID NO: 26 - protein, 6 aa, Artificial Sequence
  completely synthesized amino acid sequence
XRPYXL

SEQ ID NO: 27 - protein, 6 aa, Artificial Sequence
  completely synthesized amino acid sequence
XRPYXL

SEQ ID NO: 28 - protein, 6 aa, Artificial Sequence
  completely synthesized amino acid sequence
XRPYXL

SEQ ID NO: 29 - protein, 6 aa, Artificial Sequence
  completely synthesized amino acid sequence
XRPYXL

SEQ ID NO: 30 - protein, 6 aa, Artificial Sequence
  completely synthesized amino acid sequence
XRPYXL

SEQ ID NO: 31 - protein, 6 aa, Artificial Sequence
  completely synthesized amino acid sequence
XRPYXL

SEQ ID NO: 32 - protein, 5 aa, Artificial Sequence
  completely synthesized amino acid sequence
XPYXL

SEQ ID NO: 33 - protein, 5 aa, Artificial Sequence
  completely synthesized amino acid sequence
XPYXL

SEQ ID NO: 34 - protein, 6 aa, Artificial Sequence
  completely synthesized amino acid sequence
XRPYXL

SEQ ID NO: 35 - protein, 6 aa, Artificial Sequence
  completely synthesized amino acid sequence
XRPYXL

SEQ ID NO: 36 - protein, 6 aa, Artificial Sequence
  completely synthesized amino acid sequence
XRPYXL

SEQ ID NO: 37 - protein, 6 aa, Artificial Sequence
  completely synthesized amino acid sequence
XRPYXL

SEQ ID NO: 38 - protein, 6 aa, Artificial Sequence
  completely synthesized amino acid sequence
XRPYXL

SEQ ID NO: 39 - protein, 6 aa, Artificial Sequence
  completely synthesized amino acid sequence
XRPYXL

SEQ ID NO: 40 - protein, 6 aa, Artificial Sequence
  completely synthesized amino acid sequence
XRPYXL

SEQ ID NO: 41 - protein, 6 aa, Artificial Sequence
  completely synthesized amino acid sequence
XRPYXL

SEQ ID NO: 42 - protein, 6 aa, Artificial Sequence
  completely synthesized amino acid sequence
XRPYXL

SEQ ID NO: 43 - protein, 6 aa, Artificial Sequence
  completely synthesized amino acid sequence
XRPYXL

SEQ ID NO: 44 - protein, 6 aa, Artificial Sequence
  completely synthesized amino acid sequence
XRPYXL

SEQ ID NO: 45 - protein, 6 aa, Artificial Sequence
  completely synthesized amino acid sequence
XRPYXL

SEQ ID NO: 46 - protein, 6 aa, Artificial Sequence
  completely synthesized amino acid sequence
XRPYXL

SEQ ID NO: 47 - protein, 6 aa, Artificial Sequence
  completely synthesized amino acid sequence
XRPYXL

SEQ ID NO: 48 - protein, 6 aa, Artificial Sequence
  completely synthesized amino acid sequence
XRPYXL

SEQ ID NO: 49 - protein, 6 aa, Artificial Sequence
  completely synthesized amino acid sequence
XRPYXL

SEQ ID NO: 50 - protein, 6 aa, Artificial Sequence
  completely synthesized amino acid sequence
XRPYXL

SEQ ID NO: 51 - protein, 6 aa, Artificial Sequence
  completely synthesized amino acid sequence
XRPYXL

SEQ ID NO: 52 - protein, 6 aa, Artificial Sequence
  completely synthesized amino acid sequence
XRPYXL

SEQ ID NO: 53 - protein, 6 aa, Artificial Sequence
  completely synthesized amino acid sequence
XRPYXL

SEQ ID NO: 54 - protein, 6 aa, Artificial Sequence
  completely synthesized amino acid sequence
XRPYXL

SEQ ID NO: 55 - protein, 6 aa, Artificial Sequence
  completely synthesized amino acid sequence
XRPYXL

SEQ ID NO: 56 - protein, 13 aa, Homo sapiens
  MOD_RES (1)..(1): PYRROLIDONE CARBOXYLIC ACID
XLYENKPRRPYIL

SEQ ID NO: 57 - protein, 6 aa, Homo sapiens
RRPYIL

SEQ ID NO: 58 - protein, 6 aa, Artificial Sequence
  completely synthesized amino acid sequence
XRPYIL